SEQUENCE LISTING (1 sequence)

SEQ ID NO: 1 - RNA, 58 nt, synthetic construct
  source 1..58: mol_type = other RNA
ccgggccgctattgataatgatgttctcgagaacatcattatcaatagcggctttttg

(19) United States
(12) Patent Application Publication
(10) Pub. No.: US 2025/0152602 A1
(43) Pub. Date: May 15, 2025

(54) MODULATION OF AVERSIVE AND SALT TASTES

(71) Applicant: California Institute of Technology, Pasadena, CA (US)

(72) Inventors: Yuki Oka, Pasadena, CA (US); Yameng Zhang, Pasadena, CA (US)

(21) Appl. No.: 18/948,540

(22) Filed: Nov. 15, 2024

Related U.S. Application Data
(60) Provisional application No. 63/599,395, filed on Nov. 15, 2023.

(51) Int. Cl. A61K 31/5513; A61K 31/192; A61K 31/60; A61P 27/00; C12N 15/113; G01N 33/50

(52) U.S. Cl. CPC A61K 31/5513; A61K 31/192; A61K 31/60; A61P 27/00; C12N 15/1138; G01N 33/5023; G01N 33/5058; C12N 2310/531

(57) ABSTRACT

Disclosed herein include methods and compositions suitable for use in modulation of aversive taste tolerance. In some embodiments, ingestion of sodium and/or aversive substances is reduced. Also disclosed include methods for identifying modulators for aversive taste tolerance.

### RELATED APPLICATIONS

This application claims the benefit under 35 U.S.C. § 119(e) of U.S. Provisional Patent Application Ser. No. 63/599,395, filed Nov. 15, 2023, the content of this related application is incorporated herein by reference in its entirety for all purposes.

### STATEMENT REGARDING FEDERALLY SPONSORED R&D

This invention was made with government support under grant Nos. NS109997 and NS123918 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810002-US_SequenceListing, created Nov. 5, 2024, which is 1,959 bytes in size. The information in the electronic format of the Sequence Listing is incorporated herein by reference in its entirety.

### BACKGROUND

### Field

The present disclosure relates generally to methods and compositions for modulating aversive taste tolerance, and identification of modulators for aversive taste tolerance.

### Description of the Related Art

The hedonic value of salt fundamentally changes depending on the internal state. High concentrations of salt induce innate aversion under sated states, whereas such aversive stimuli transform into appetitive ones under sodium depletion. Neural mechanisms underlying this state-dependent salt valence switch are poorly understood. There is still a need for identifying modulators for aversive taste tolerance, as well as developing methods and composition for modulating aversive taste tolerance in subjects in need thereof.

### SUMMARY

Disclosed herein include methods of reducing aversive taste tolerance in a subject in need thereof. In some embodiments, the method comprises: inhibiting a plurality of prostaglandin receptor type 3 (Ptger3)-positive neurons in the lamina terminalis (LT^{Ptger3}-neurons) of the subject, thereby reducing aversive taste tolerance in the subject.

In some embodiments, reducing aversive taste tolerance in the subject comprises reducing ingestion of sodium, one or more aversive substances, or a combination thereof by the subject. In some embodiments, the sodium is at a concentration of less than 120 mM. In some embodiments, the sodium is at a concentration of greater than 300 mM. In some embodiments, reducing aversive taste tolerance in the subject comprises reducing ingestion of sodium and one or more aversive substances by the subject, and wherein the sodium is at a concentration of less than 120 mM. In some embodiments, the one or more aversive substances comprises one or more aversive minerals, one or more bitter-tasting substances, one or more sour-tasting substances, or any combination thereof. In some embodiments, the one or more aversive minerals comprise potassium chloride (KCl), calcium chloride (CaCl₂), and/or magnesium chloride (MgCl₂). In some embodiments, the one or more bitter-tasting substances comprise polyphenols, alkaloids, terpenoids, saponins, amino acids, bitter peptides, or any combination thereof. In some embodiments, the one or more bitter-tasting substances comprise quinine. In some embodiments, the one or more sour-tasting substances comprise lactic acid, citric acid, malic acid, acetic acid, or any combination thereof.

The method can comprise determining sodium intake in the subject before inhibiting the plurality of LT^{Ptger3}-neurons of the subject. The method can comprise determining sodium intake in the subject after inhibiting the plurality of LT^{Ptger3}-neurons of the subject. In some embodiments, the aversive taste tolerance of the subject is reduced by at least 25% in the subject, e.g., as assessed by ingestion of the one or more aversive substances and/or sodium by the subject.

In some embodiments, the LT^{Ptger3}-neurons are in the subfornical organ of the LT (SFO^{Ptger3}-neurons), the vascular organ of the LT (OVLT^{Ptger3}-neurons), or both. In some embodiments, the SFO^{Ptger3}-neurons are GLUT1-expressing excitatory neurons. In some embodiments, the SFO^{Ptger3}-neurons do not express GLUT2, GLUT3, GLUT4, and/or GLUT5.

In some embodiments, inhibiting the plurality of LT^{Ptger3}-neurons of the subject comprises administration of a Ptger3-inhibitor to the subject. In some embodiments, the Ptger3-inhibitor inhibits Ptger3 expression, activity, or both. In some embodiments, the Ptger3-inhibitor inhibits activity of a prostaglandin. In some embodiments, the prostaglandin is Prostaglandin E2 (PGE2). In some embodiments, the Ptger3-inhibitor is a small molecule, an antibody, or a nucleic acid. In some embodiments, the nucleic acid is an anti-sense RNA. In some embodiments, the anti-sense RNA comprises a microRNA (miRNA), a precursor microRNA (pre-miRNA), a small interfering RNA (siRNA), a short-hairpin RNA (shRNA), precursors thereof, derivatives thereof, or a combination thereof. In some embodiments, the shRNA comprises the sequence of SEQ ID NO: 1. In some embodiments, the nucleic acid is complexed or associated with one or more lipids or lipid-based carriers, thereby forming liposomes, lipid nanoparticles (LNPs), lipoplexes, and/or nanoliposomes, e.g., encapsulating the nucleic acid composition. In some embodiments, the nucleic acid comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the Ptger3-inhibitor is an anti-inflammatory agent. In some embodiments, the anti-inflammatory agent is a non-steroidal anti-inflammatory drug (NSAID). In some embodiments, the NSAID comprises ibuprofen, dexibuprofen, fenoprofen, flurbiprofen, ketoprofen, oxaprozin, naproxen, dexketoprofen, loxoprofen, aspirin, salicylic acid, diflunisal, salsalate, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or any combination thereof. In some embodiments, the Ptger3-inhibitor is administered to the subject orally or intravenously.

In some embodiments, inhibiting the plurality of LT^{Ptger3}-neurons of the subject comprises optogenetic inhibition or chemogenetic inhibition. In some embodiments, the optogenetic inhibition or the chemogenetic inhibition comprises inhibiting the plurality of LT^{Ptger3}-neurons by a conditional ion modulator. In some embodiments, inhibiting the plurality of LT^{Ptger3}-neurons comprises: administering a nucleic acid encoding the conditional ion modulator to the subject, wherein the conditional ion modulator is activated in response to a stimulus or agonist; and applying stimulus or agonist of the conditional ion modulator to the subject, causing the activation of the conditional ion modulator, thereby inhibiting the plurality of LT^{Ptger3}-neurons. In some embodiments, the conditional ion modulator comprises a chloride conducting channelrhodopsin (ChloC) or a halorhodopsin (HR), and the stimulus comprises an optical stimulus. In some embodiments, the conditional ion modulator comprises a designer receptor exclusively activated by designer drug (DREADD), and the agonist is clozapine-N-oxide (CNO). In some embodiments, the reduction of aversive taste tolerance is detectable within 20 minutes of the onset of the chemogenetic inhibition. In some embodiments, the nucleic acid is administered to the subject in an adeno-associated viral (AAV) vector.

The method can comprise identifying the subject as a subject in need of reducing aversive taste tolerance. In some embodiments, the subject is a subject suffering from a kidney disorder, kidney damage, a cardiovascular disease, high blood pressure, obesity, edema, left ventricular hypertrophy, stroke, or a combination thereof. In some embodiments, the kidney disorder is a chronic kidney disease or kidney failure. In some embodiments, inhibiting the plurality of LT^{Ptger3}-neurons does not reduce appetitive drive for sodium in the subject.

Disclosed herein include methods of increasing aversive taste tolerance in a subject in need thereof. In some embodiments, the method comprises: stimulating a plurality of prostaglandin receptor type 3 (Ptger3)-positive neurons in the lamina terminalis (LT^{Ptger3}-neurons) of the subject, thereby increasing aversive taste tolerance in the subject.

In some embodiments, increasing aversive taste tolerance in the subject comprises increasing ingestion of sodium, one or more aversive substances, or a combination thereof by the subject. In some embodiments, the sodium is at a concentration of about less than 120 mM. In some embodiments, the sodium is at concentration of greater than 300 mM. In some embodiments, increasing aversive taste tolerance in the subject comprises increasing ingestion of sodium and one or more aversive substances by the subject, and wherein the sodium is at a concentration of less than 120 mM. In some embodiments, the one or more aversive substances comprises one or more aversive minerals, one or more bitter-tasting substances, one or more sour-tasting substances, or any combination thereof. In some embodiments, the one or more aversive minerals comprise potassium chloride (KCl), calcium chloride (CaCl₂), and/or magnesium chloride (MgCl₂). In some embodiments, the one or more bitter-tasting substances comprise polyphenols, alkaloids, terpenoids, saponins, amino acids, bitter peptides, or any combination thereof. In some embodiments, the one or more bitter-tasting substances comprise quinine. In some embodiments, the one or more sour-tasting substances comprise lactic acid, citric acid, malic acid, acetic acid, or any combination thereof.

The method can comprise determining sodium intake in the subject before stimulating the plurality of LT^{Ptger3}-neurons of the subject. The method can comprise determining sodium intake in the subject after stimulating the plurality of LT^{Ptger3}-neurons of the subject. In some embodiments, the aversive taste tolerance of the subject is increased by at least 25% in the subject, e.g., as assessed by ingestion of the one or more aversive substances and/or sodium by the subject.

In some embodiments, the LT^{Ptger3}-neurons are in the subfornical organ of the LT (SFO^{Ptger3}-neurons), the vascular organ of lamina terminalis (OVLT^{Ptger3}-neurons), or both. In some embodiments, the SFO^{Ptger3}-neurons are GLUT1-expressing excitatory neurons. In some embodiments, the SFO^{Ptger3}-neurons do not express GLUT2, GLUT3, GLUT4, and/or GLUT5.

In some embodiments, stimulating the plurality of LT^{Ptger3}-neurons of the subject comprises administration of a Ptger3-activator to the subject. In some embodiments, the Ptger3-activator increases Ptger3 expression, activity, or both. In some embodiments, the Ptger3-activator increases activity of a prostaglandin. In some embodiments, the prostaglandin is Prostaglandin E2 (PGE2). In some embodiments, the Ptger3-activator is a small molecule or a peptide. In some embodiments, the peptide comprises at least a portion of PGE2 or a functional fragment thereof, or a PGE2 mimetic. In some embodiments, the Ptger3-activator is administered to the subject orally or intravenously.

In some embodiments, stimulating the plurality of LT^{p8}° °-neurons of the subject comprises optogenetic stimulation or chemogenetic stimulation. In some embodiments, the optogenetic stimulation or the chemogenetic stimulation comprises inhibiting the plurality of LT^{Ptger3}-neurons by a conditional ion modulator. In some embodiments, stimulating the plurality of LT^{Ptger3}-neurons comprises: administering a nucleic acid encoding the conditional ion modulator to the subject, wherein the conditional ion modulator is activated in response to a stimulus or agonist; and applying the stimulus or agonist of the conditional ion modulator to the subject, causing the activation of the conditional ion modulator, thereby stimulating the plurality of LT^{Ptger3}-neurons. In some embodiments, the conditional ion modulator comprises a channelrhodopsin-2 (ChR2), and the stimulus comprises an optical stimulus. In some embodiments, the conditional ion modulator comprises a designer receptor exclusively activated by designer drug (DREADD), and the agonist is clozapine-N-oxide (CNO). In some embodiments, the increase in aversive taste tolerance is detectable within 20 minutes of the onset of the chemogenetic stimulation.

In some embodiments, the subject is a subject suffering from hyponatremia, excessive sweating, or a combination thereof. In some embodiments, stimulating the plurality of LT^{Ptger3}-neurons increases dopamine signaling in the central nervous system of the subject. In some embodiments, stimulating the plurality of LT^{Ptger3}-neurons does not increase appetitive drive for sodium in the subject.

Disclosed herein include methods of identifying a modulator of aversive taste tolerance. In some embodiments, the method comprises: (a) contacting a candidate compound with a plurality of prostaglandin receptor type 3 (Ptger3)-positive neurons in the lamina terminalis (LT^{Ptger3}-neurons) to determine an electrophysiological response in the LT^{Ptger3}-neurons; (b) identifying the candidate compound as a modulator of the LT^{Ptger3}-neurons if the electrophysiological response in the LT^{Ptger3}-neurons contacted with the candidate compound is altered as compared to the electrophysiological response in the LT^{Ptger3}-neurons prior to contacting with the candidate compound; (c) administering the identified modulator of the LT^{Ptger3}-neurons to a subject; (d) assessing the change in valence toward sodium and/or one or more aversive substances of the subject in response to the administration of the identified modulator of the LT^{Ptger3}-neurons; and (e) identifying the identified modulator of the LT^{Ptger3}-neurons as a modulator for aversive taste tolerance if the identified modulator of the LT^{Ptger3}-neurons changes the valence toward sodium and/or one or more aversive substances of the subject compared to a control.

In some embodiments, the electrophysiological response is measured by an assay selected from the group consisting of a Ca²⁺ influx assay, a patch clamp assay, a calcium mobilization assay, a calcium imaging assay, an electrical signal detection assay, an assay based on a fluorescent calcium sensor, or a combination thereof. In some embodiments, contacting the candidate compound with the plurality of LT^{Ptger3}-neurons comprises administering the candidate compound to a subject comprising the LT^{Ptger3}-neurons via injection or via oral administration. In some embodiments, the modulator is a suppressor for aversive taste tolerance and step (d) comprises identifying the candidate compound as a suppressor for aversive taste tolerance if the candidate compound reduces valence toward sodium and/or one or more aversive substances of the subject compared to a control; or wherein the modulator is an enhancer for aversive taste tolerance and step (d) comprises identifying the candidate compound as an enhancer for aversive taste tolerance if the candidate compound enhances valence toward sodium and/or one or more aversive substances of the subject compared to a control. In some embodiments, the candidate compound is a small molecule, a peptide, a nucleic acid, or a combination thereof. In some embodiments, administering the identified modulator of the LT^{Ptger3}-neurons to the subject is via injection or via oral administration. In some embodiments, the subject comprising the LT^{Ptger3}-neurons and the subject administered with the identified modulator of the LT^{Ptger3}-neurons are different subject. In some embodiments, the subject is a mammal. In some embodiments, the subject is a mouse, a rat, or a human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1D display non-limiting exemplary data related to independent regulation of appetitive drive and tolerance toward salt. FIG. 1A displays a consumption assay under sodium depleted (−Sodium, left), osmotic thirst (Thirst, middle), or sated (Sated, right) conditions. Animals were tested with pure water, KCl (500 mM), low salt (60 mM NaCl), high salt (500 mM NaCl), low salt+440 mM KCl, low salt+40 mM CaCl₂, and low salt+40 mM MgCl₂, and low salt+0.25 mM quinine (n=6-21 mice). Shown in FIG. 1B is a diagram of state-dependent salt tolerance. Sodium-depleted animals accept aversive high salt or low salt with additional aversive stimuli. Conversely, sated or thirsty animals reject the same solutions. FIG. 1C shows a bimodal regulation model of salt consumption under sodium depletion. Shown in FIG. 1D is a comparison of pre-LC^{Pdyn}-stimulated and sodium-depleted conditions. Left, a scheme of acute photostimulation of pre-LC^{Pdyn} neurons. Middle and right, Cumulative consumption curves of pre-LC^{Pdyn}-stimulated (blue) and sodium depleted (red) conditions during a 30-min session. Low and high salt (60 and 500 mM NaCl) was accepted by both pre-LC^{Pdyn}-stimulated and sodium-depleted animals. Only sodium-depleted animals tolerated low salt with KCl or quinine but not photostimulated animals (n=9-15 mice). Data are expressed as mean±SEM, *p<0.05, **p<0.01, ***p<0.001.

FIG. 2A-FIG. 2I display non-limiting exemplary data showing sodium depletion activates selective excitatory neuron types in the LT. Shown in FIG. 2A are representative images of Fos (red) immunofluorescence signals in the forebrain SFO, and hindbrain pre-LC under sodium depletion and sated states. The SFO was counterstained by an excitatory marker nNos (blue) while the pre-LC was counterstained by Foxp2 (blue). The locations of the SFO and pre-LC are −5.52 and −0.7 mm relative to Bregma, respectively. Shown in FIG. 2B is stimulus-to-cell-type scRNA-seq mapping of neurons activated under sodium depletion. Left, experimental diagram of scRNA-seq. Right, UMAP embedding of SFO neurons. Data from sodium-depleted and sated mice were integrated using CCA alignment (sated n=3380 neurons, sodium depleted n=4439 neurons). FIG. 2C displays UMAP embedding for Fos log-normalized expression (red) in SFO excitatory neurons under sated (left) and sodium-depleted conditions (right). Shown in FIG. 2D is identification of Glut1-enriched genes. Log-normalized average gene expression in Glut1 cluster was compared to other excitatory neural clusters. Glut1- and Glut2-5-enriched genes were shown outside the dotted lines (Table 1). FIG. 2E shows UMAP embedding of Ptger3 log-normalized expression demonstrates faithful expression of Ptger3 in the Glut1 cluster. Shown in FIG. 2F are violin plots of Fos (red) and Ptger3 (blue) under sated (left) and sodium-depleted (right) conditions. Ptger3 is selectively expressed in Glut1 and Glut1 was specifically activated during sodium depletion in the SFO. FIG. 2G displays SeqFish analysis of the SFO. Left, spatial distribution of all major cell types in representative anterior (top) and posterior (bottom) sections. After segmentation, cells were color-coded as indicated. Right, A total of 1,567 cells from five anterior sections and 1,614 cells from five posterior sections are quantified. Percentage of individual cell type is presented in the pie chart. Exc. Neurons, excitatory neurons; Inh. Neurons, inhibitory neurons; LT Astro, LT astrocytes; Astro, astrocytes; LT endo, LT endothelial cells; Endo, Endothelial cells; VSMCs, vascular smooth muscle cells; Ependy, Ependymal cells; Oligo, oligodendrocytes. In FIG. 2H, spatial distribution of all cells is plotted according to the original cell center coordinates. Excitatory neurons are highlighted in colors (red and blue). The Glut1/Ptger3 cluster (red) is separate from other excitatory neuron clusters (blue). Neurons in gray represent excitatory neurons showing gene expression of multiple cluster markers. FIG. 2I displays violin plots showing the log-normalized expression of Fos (red) and Ptger3 (blue) in SFO excitatory neurons. Fos expression was found specifically in the Glut1 cluster under sodium depletion. Scale bar, 100 μm (FIG. 2A), 50 μm (FIG. 2G-FIG. 2H). See also Table 1.

FIG. 3A-FIG. 3F display non-limiting exemplary data showing SFO^{Ptger3} neurons mediate salt tolerance. FIG. 3A shows the genetic structure of the Ptger3^{Cre} mouse line (top). Light and dark blue shades indicate UTRs and exons of Ptger3. Cre is inserted into the first exon, resulting in Ptger3 disruption. In situ hybridization showing co-expression of Ptger3 (green) and Cre:GFP (red) expression (bottom); 86% of Ptger3+ cells expressed Cre:GFP and 83% of Cre:GFP+neurons expressed Ptger3 (n=9 sections from 7 mice). FIG. 3B displays representative Fos immunofluorescence signals (red) under distinct internal states and noxious stimulus. SFO^{Pger3} neurons are labeled green. Activation was highly selective under sodium depletion with a minor activation level induced by inflammation. Pie charts display the percentage of Fos+/Ptger3+ cells in all Fos+ cells. FIG. 3C displays data related to an appetitive-drive test. Photostimulation of SFO^{Ptger3} neurons did not induce salt consumption. Middle, histological validation of Fos immunofluorescence signals (red) after photostimulation of ChR2-expressing SFO^{Pger3} neurons (green). Bottom graphs show the averaged lick numbers of water, low (60 mM), medium (250 mM) and high (500 mM) concentrations of sodium at 20 Hz stimulation and the averaged lick numbers of high salt with different stimulation frequency (n=4-11 mice). FIG. 3D displays data related to a high-salt tolerance test. Left, representative raster plots of licking behavior toward high salt during a 5-sec session from a sated+photostimulated (black), thirsty (blue), or thirsty+photostimulated (red) animal. Right, quantified lick numbers without (blue, first bar in each solution) or with (red, second bar for each solution) photostimulation across different concentrations of sodium: water, 60 mM, 250 mM, 500 mM (n=4-12 mice). FIG. 3E displays data related to an aversive-taste-tolerance test. The number of licks without (blue, first bar for each solution) or with (red, second bar for each solution) photostimulation for low salt supplemented with KCl or quinine (n=6 mice). FIG. 3F displays data showing LT^{Ptger3} neurons are required for normal salt tolerance. Left, a diagram of chemogenetic loss-of-function experiments. Middle, the number of licks for water or high salt during a 30-min session with vehicle (grey) or CNO (red, second bar for each solution) injection under osmotic thirst (n=5-7 mice). Right, the same behavioral analyses under sodium depletion for low salt, low salt with KCl, or quinine (n=7 mice). Data are expressed as mean±SEM, *p<0.05, ***p<0.001. Scale bar, 25 μm (magnified images), 50 μm (FIG. 3A), 100 μm (FIG. 3B-FIG. 3C).

FIG. 4A-FIG. 4E display non-limiting exemplary data related to parallel and independent activation of fore- and hindbrain circuits under sodium depletion. Shown in FIG. 4A are two distinct circuit models between fore- and hindbrain neurons related to sodium ingestion. Left, a parallel model where sodium depletion independently activates SFO^{Ptger3} and pre-LC^{Pdyn} neurons. Right, a serial-activation model depicting the hierarchical relationship between the two neural circuits. FIG. 4B displays data related to testing pre-LC^{Pdyn} →SFO^{Ptger3} projections. AAV-DIO-ChR2-EYFP was transduced in pre-LC^{Pdyn} neurons. Top, representative images showing axonal projections to the ventral bed nucleus of stria terminalis (vBNST) and SFO. Note that the vBNST is a known downstream area of pre-LC^{Pdyn} neurons. Bottom, similarly, photostimulation-induced Fos immunofluorescence signals were observed in the BNST but not SFO. Right, the number of Fos+ cells was compared between SFO (red) and pre-LC (grey, n=4 sections from 4 mice). FIG. 4C displays data related to testing SFO^{Ptger3} →pre-LC^{Pdyn} projections. Top, SFO^{Ptger3} neurons showed no projection to the pre-LC when compared to the MnPO, a known downstream target of the SFO. Bottom, no activation was found in pre-LC^{Pdyn} neurons after photostimulating SFO^{Pger3} neurons. Right, the number of Fos+ cells by photostimulation (n=4-6 sections from 3 mice). FIG. 4D displays data showing activation of SFO^{Ptger3} neurons under sodium depletion did not rely on functioning pre-LC^{Pdyn} neurons. AAV-FLEX-taCasp3-TEVP was injected into the pre-LC bilaterally in Pdyn^{Cre} or wild-type mice. Middle, representative images of sodium-depletion-induced Fos in the SFO and pre-LC. Right, the numbers of Fos+ cells in the SFO and pre-LC were quantified in transgenic (Cre, red, n=4-6 sections from 3 mice) and wild-type mice (WT, grey,n=5-8 sections from 4 mice). FIG. 4E displays data related to pre-LC activation with ablated LT^{Ptger3} neurons. AAV-FLEX-taCasp3-TEVP was injected to the SFO and OVLT in Ptger3^{Cre/wt} or wild-type mice. Middle, representative images of sodium-depletion-induced Fos. Right, quantified Fos+ cells in the SFO and pre-LC. Ablation of LT^{Ptger3} neurons had no effect on the pre-LC activity (red, n=4 sections from 2 mice; grey, n=4 sections from 3 mice for SFO, and red, 4 sections from 2 mice and 5 sections from 3 mice for pre-LC). Data are shown as mean SEM, *p<0.05, **p<0.01, ***p<0.001. Scale bar, 100 m.

FIG. 5A-FIG. 5G display non-limiting exemplary data related to selective modulation of aversive taste saliency by SFO^{Ptger3} neurons. FIG. 5A displays data related to dose-dependent behavioral aversion toward bitter tastes. Left, the number of licks was quantified for water, 0.125, and 0.25 mM quinine under osmotic thirst conditions in the absence (black) or presence (red) of photostimulation to SFO^{Ptger3} neurons. Right, preference changes are shown as a ratio by calculating the lick numbers with photostimulation divided by those without photostimulation (n=5-8 mice). Lick number of water was reanalyzed from FIG. 3D. FIG. 5B displays similar analyses as (FIG. 5A) using sour taste. Water, 10, and 20 mM citric acid were used to quantify taste preference (n=8-11 mice). Lick number of water was reanalyzed from FIG. 3D. FIG. 5C displays data showing acute stimulation of SFO^{Ptger3} neurons did not change preference toward attractive tastant under food deprivation. Average lick numbers for water, 0.5, 1, and 2 mM AceK are shown without (black) or with (red) photostimulation (n=4-7 mice). FIG. 5D displays data related to dose-dependent behavioral aversion toward capsaicin, a non-taste oral aversive compound. 0, 0.3, and 1 M capsaicin were used to calculate avoidance curve (n=4-5 mice). FIG. 5E displays a schematic of saliency modulation by SFO^{Ptger3} neurons was specific toward aversive stimuli through the taste system. The effect was not generalized to non-oral aversive stimuli. As shown in FIG. 5F, stimulation of Ptger3 neurons alleviate aversive taste response under hunger. Shown are raster plots from representative food-deprived mice to sweet (2 mM AceK), sweet with bitter (2 mM AceK with 0.125 mM quinine), and the same mixture with photostimulation. With the photostimulation of SFO^{Ptger3} neurons, the bitter taste was tolerated. FIG. 5G displays data showing the effect of photostimulation of SFO^{Ptger3} neurons on glucose (500 mM), AceK (2 mM) and monopotassium glutamate+inosine monophosphate (30 mM+0.6 mM) supplemented with bitter. The average lick numbers toward pure attractive tastants (blue), mixture with 0.125 mM quinine (grey), and together with photostimulation (red) are shown (n=7-10 mice). Data are shown as mean±SEM, *p<0.05, **p<0.01, ***p<0.001.

FIG. 6A-FIG. 6F display non-limiting exemplary data showing Ptger3 in the SFO is required for salt tolerance. As shown in FIG. 6A, fluorescence in situ hybridization validates the lack of Ptger3 transcripts in the SFO in homozygous Ptger3^{Cre/Cre} (Ptger3−/−) animals compared to heterozygous Ptger3^{Cre/wt} (Ptger3+/−) animals. 89% of Ptger3 signals were abolished in Ptger3^{Cre/Cre} mice (n=4 sections from 3 mice) compared to Ptger3^{Cre/wt} (n=5 sections from 3 mice). FIG. 6B (Left) displays representative images of the SFO and pre-LC under sodium depletion in Ptger3^{Cre/wt} (grey) and Ptger3^{Cre/Cre} (red) animals. Sodium depletion activated the SFO in a Ptger3-dependent manner but Fos immunofluorescence signals in pre-LC were unaffected. FIG. 6B (Right) shows quantification of the cell activation (grey, n=4 sections from 4 mice; red, n=5 sections from 5 mice for SFO, and grey, n=7 sections from 4 mice; red, n=6 sections from 3 mice for pre-LC). FIG. 6C displays data showing aversion tolerance is abolished in Ptger3^{Cre/Cre} mice. The graph shows the number of licks toward water, low salt (60 mM NaCl), high salt (500 mM NaCl), and low salt with KCl or quinine in Ptger3^{Cre/w (grey) and Ptger}3^{Cre/Cre} (red) mice under sodium depletion (n=4-10 mice). As shown in FIG. 6D, fluorescence in situ hybridization validates the lack of Ptger3 transcripts in the Ptger3-shRNA-injected animals. Compared to scramble-shRNA-injected animals, 87% of Ptger3 signals were abolished in Ptger3-shRNA-injected mice (n=4 sections from 2 mice) compared to scramble-shRNA-injected mice (n=4 sections from 2 mice). Shown in FIG. 6E (Left) are representative images of the SFO and pre-LC under sodium depletion in scramble-(grey) and Ptger3-(red) shRNA-injected mice. Shown in FIG. 6E (Right) is quantification of the cell activation (grey, n=5 sections from 5 mice; red, n=5 sections from 5 mice). FIG. 6F displays data showing aversion tolerance is abolished in Ptger3 KD mice. Left, a diagram of gene knockdown and behavioral paradigm. Right, consumption of the same solutions as FIG. 6C was tested in scramble-(grey) and Ptger3-(red) shRNA-injected mice (n=5-13 mice). Data are expressed as mean±SEM, *p<0.05, **p<0.01, ***p<0.001. Scale bar, 25 μm (FIG. 6A right, and FIG. 6D), 100 μm (FIG. 6A left, FIG. 6B, and FIG. 6E).

FIG. 7A-FIG. 7H display non-limiting exemplary data related to functional roles of PGE2-Ptger3 signaling for salt taste modulation. FIG. 7A shows ELISA measurement of the circulating PGE2 level. PGE2 was measured from sated (grey), and sodium depleted/repleted (red), and formalin-injected (blue) animals (n=6-9 mice). FIG. 7B displays data related to rapid PGE2 access to the SFO. Ex vivo imaging of the SFO was performed after intravenous injection of PGE2-AMCA. Top, experimental diagram and the chemical structure of a synthesized PGE2-AMCA. Bottle left, representative images showing fluorescence of PGE2-AMCA in acutely dissected SFO. Fluorescence levels were quantified across indicated white lines. Bottom middle, representative traces of relative fluorescence levels in vehicle- or PGE2-AMCA-injected animals. Bottom right, quantification of the relative fluorescence level (n=7 mice for PBS, and 9 mice for PEG2-AMCA). Shown in FIG. 7C are photometry recording from SFO^{Ptger3} neurons. Shown are calcium dynamics and quantified responses (normalized ΔF/F) from SFO^{Ptger3} neurons after subcutaneous injection of vehicle (grey) and PGE2 (red, n=6 recordings from 5 mice for PGE2, and 7 recordings from 5 mice for vehicle). As shown in FIG. 7D, peripheral injection of PGE2 induces salt tolerance in a Ptger3-dependent manner. High-salt consumption after subcutaneous vehicle (grey) or PGE2 (red, second bar each condition) injection in Ptger3^{Cre/wt} and Ptger3^{Cre/Cre} animals was analyzed (n=4-5 mice). PGE2 did not induce a drive to consume salt under sated conditions. Under thirst condition, Ptger3^{Cre/wt}, but not Ptger3^{Cre/Cre}, animals showed enhanced salt tolerance with PGE2 injection. FIG. 7E displays data showing Ptger3 KD mice did not tolerate high salt upon PGE2 administration. Scramble-(grey) and Ptger3-(red) shRNA-injected mice were tested in high salt consumption assay (n=5-10 mice). As shown in FIG. 7F, anti-inflammatory drugs (NSAIDs) partially decreased salt tolerance in sodium depletion. Sodium-depleted mice supplemented with ibuprofen (drinking water) and aspirin (i.p. injection) showed decreased tolerance toward high salt (n=4-5 mice). FIG. 7G displays data related to enhanced dopamine signals toward high salt with PGE2 injection. Left, dopamine release in the NAc was monitored with dLight1.3b during high-salt consumption was measured. Middle and right, averaged dLight1.3b dynamics and quantified data are shown under sodium depletion, thirst, and thirst with PGE2 subcutaneous injection. Lick frequency is shown under dLight traces (n=8-9 mice). As shown in FIG. 7H, activation of SFO^{Ptger3} neurons induced dopamine release during high-salt consumption. Left, an experimental diagram for virus injection and salt consumption assay. Middle and right, averaged dLight1.3b dynamics and quantified data are shown after i.p. vehicle (grey) or CNO (red) injection (n=8 mice). Data are expressed as mean±SEM, *p<0.05, **p<0.01, ***p<0.001. Scale bar, 100 m.

FIG. 8A-FIG. 8F display non-limiting exemplary data showing salt tolerance is enhanced under sodium depletion but not under thirst or pre-LC^{Pdyn}-induced appetite. Shown in FIG. 8A is a diagram of appetitive and aversive salt taste pathways. Low concentrations of salt only activate the appetitive ENaC pathway, and animals exhibit a behavioral attraction. Higher concentrations of salt recruit additional aversive taste pathways (bitter and sour), driving strong behavioral aversion despite the activation of appetitive ENaC pathway. FIG. 8B-FIG. 8C display cumulative consumption curves under osmotic thirst and sodium-depleted conditions from the data in FIG. 1A. Shown in FIG. 8D are the total lick numbers were quantified from the data in FIG. 1D. Unstimulated control data (grey) were shown together. −Sodium is the first bar for each solution, and +Stim is the second bar for each condition. FIG. 8E (Top) displays comparison of low- and high-salt consumption in pre-LC^{Pdyn}-stimulated animals (blue) and sodium-depleted animals (red). FIG. 8E (Bottom) shows consumption ratio between low salt and high salt. pre-LC^{Pdyn}-stimulated animals preferred low salt while sodium-depleted animals consumed significantly more high salt (n=12-15 mice). The data were reanalyzed from FIG. 1D. FIG. 8F displays data showing sodium-selective, behavioral attraction is mediated by pre-LC^{Pdyn} neurons. The number of licks toward high salt (500 mM NaCl) was quantified during pre-LC^{Pdyn} photostimulation with or without 30 M amiloride. High salt consumption induced by pre-LC^{Pdyn} neurons was ENaC dependent (n=5-6 mice). −Stim is the first bar for each condition, and +Stim is the second bar for each condition. Data are expressed as mean±SEM, *p<0.05, **p<0.01, ***p<0.001.

FIG. 9A-FIG. 9D display non-limiting exemplary data related to neural activation of SFO and OVLT under sodium depletion and hypovolemia. FIG. 9A displays representative images of OVLT activation in sated (top) and sodium-depleted (bottom) conditions. The OVLT was visualized with nNos (blue) and Fos (red) activation was compared between conditions. FIG. 9B shows violin plots of Fos (red) and Ptger3 (blue) in the SFO under hypovolemia conditions. Ptger3-expressed neurons were a partial population active in hypovolemia. FIG. 9C displays representative Fos immunofluorescence signals (red) under hypovolemia in the SFO. Ptger3 neurons are labeled in green. FIG. 9D displays data related to spatial distribution of all cells according to the original cell center coordinates. Individual excitatory neuron types are highlighted in colors (as indicated). Scale bar, 25 μm (FIG. 9C, magnified images), 100 μm (FIG. 9A, FIG. 9C, and FIG. 9D).

FIG. 10A-FIG. 10J display non-limiting exemplary data related to functional characterization of SFO^{Ptger3} neurons. FIG. 10A displays data related to characterization of Ptger3^{Cre} transgenic line in the whole brain. Shown are representative GFP signals from a Ptger3^{Cre/wt} animal. Strong GFP signals were found in the SFO, island of Calleja major (ICjM), preoptic area (POA), paraventricular thalamus (PVT), cortex, periaqueductal gray (PAG), and raphe pallidus (RPa). FIG. 10B shows quantified Fos+ cells in the SFO that overlapped with Ptger3-GFP signals. Shown are the ratio of activated Ptger3+ neurons divided by total Fos+ cells (n=2-6 sections from 2-5 mice). Shown in FIG. 10C are representative images of Ptger3-Cre:GFP (green) overlap with AAV-DIO-ChR2-mCherry (red). 88% of mCherry+neurons expressed Ptger3-Cre:GFP, and 72% of Cre:GFP+neurons were mCherry+(n=7 sections from 6 mice). FIG. 10D displays a chart of the relationship between virus expression level and salt tolerance. The number of ChR2+ neurons per section and lick numbers toward high salt are shown for individual animals tested. Note that salt tolerance and the number of ChR2+ neurons have a high correlation coefficient (n=16 mice). FIG. 10E shows control experiments for ChR2 photostimulation using mCherry (n=5 mice). FIG. 10F displays data showing amiloride did not affect salt tolerance induced by SFO^{Ptger3} Unlike pre-LC^{Pdyn} neurons, SFO^{Ptger3}-induced tolerance is independent of the ENaC pathway (n=5 mice). −Stim is the first bar for each condition, and +Stim is the second bar for each condition. FIG. 10G displays data related to sodium-depletion-induced tolerance in thirsty mice. Mice were subjected to osmotic thirst (grey, first bar each solution) and osmotic thirst+sodium depletion (red, second bar each solution), and the number of licks toward 250 and 500 mM NaCl were quantified (n=6-12 mice). The thirst data for 250 mM NaCl included the data from FIG. 3D. FIG. 10H displays a representative image of Ptger3 (green) and Fos (red) immunofluorescence signals in the OVLT. As shown in FIG. 10I, LT^{Ptger3} neurons are required for normal salt tolerance under hypovolemia. The number of licks toward 500 mM NaCl during a 30-min trial with vehicle (grey, first bar) or CNO (red, second bar) injection under hypovolemia (n=6 mice). FIG. 10J displays data showing SFO^{Ptger3} neurons are required for normal salt tolerance. Left, a diagram of chemogenetic loss-of-function experiments. AAV-DIO-hM4Di was injected into the SFO. Right, the number of licks for low salt, low salt with KCl or quinine was quantified during a 30-min session under sodium depletion. Before behavioral experiments, animals received intraperitoneal injection of either vehicle (grey, first bar each solution) or CNO (red, second bar each solution) (n=6 mice). Data are expressed as mean±SEM, *p<0.05, **p<0.01, ***p<0.001. Scale bar, 500 μm (FIG. 10A), 100 μm (FIG. 10C and FIG. 10H).

FIG. 11A-FIG. 11G display non-limiting exemplary data showing specificity of sensory modulation by SFO^{Ptger3} neurons. FIG. 11A displays data showing Trpm5−/− animals that lack bitter, sweet, and umami tastes showed elevated tolerance toward aversive tastes. Left, consumption of water and 60 mM NaCl during a 30-min session under osmotic thirst in wild-type (grey, first bar each solution) and Trpm5−/− mice (red, second bar each solution, n=3-14). Right, consumption of 500 mM NaCl, 60 mM NaCl with 440 mM KCl, and 60 mM NaCl with 0.25 mM quinine during a 30-min session under osmotic thirst in wild-type (grey) and Trmp5−/− mice (red) (n=3-14). For wild-type control, the data were reanalyzed from FIG. 1A. FIG. 11B displays data showing that stimulation of pre-LC^{Pdyn} neurons did not affect behavioral aversion toward bitter tastes. The number of licks was quantified for water, 0.125 and 0.25 mM quinine under osmotic thirst conditions in the absence (black) or presence (red) of photostimulation to pre-LC^{Pdyn} neurons (n=4-6 mice). FIG. 11C displays data showing learned taste aversion was not alleviated by the activity of SFO^{Ptger3} neurons. Left, a conditioned-taste-avoidance paradigm toward AceK (2 mM) by LiCl injection. Right, consumption of AceK before (grey) and after (−stim, blue; +stim, red) conditioning (n=6). As shown in FIG. 11D-FIG. 11E, various aversive stimuli were presented to animals under sated, sodium-depleted, and SFO^{Ptger3}-stimulated conditions. Animals were subjected to noxious heat on a hot plate (FIG. 11D), and von Frey test (FIG. 11E). Neither sodium depletion nor SFO^{Ptger3}-stimulation blunted general aversive or tactile responses (n=4-12 mice). FIG. 11F displays data related to an acute and inflammatory pain test. Left, a histogram showing the time spent paw-licking after formalin injection plotted in 5-min bins in sated and sodium-depleted conditions. Right, a cumulative time of paw-licking during the acute pain period (0-5 min after formalin injection) and the inflammatory pain period (15-45 min after formalin injection). The data were compared between sated (grey, first bar for each of AP and IP) and sodium-depleted (red, second bar for each of AP and IP) groups (n=4 mice). FIG. 11G displays data showing photostimulation of SFO^{Ptger3} neurons had no effects on pain responses. Left, a histogram showing the time spent paw-licking after formalin injection without (grey, first bar for each of AP and IP) or with (red, second bar for each of AP and IP) photostimulation. Right, quantified cumulative time of paw-licking (n=4 mice). Data are expressed as mean±SEM, *p<0.05, **p<0.01, ***p<0.001. Scale bar, 100 m.

FIG. 12A-FIG. 12C display non-limiting exemplary data showing Ptger3 in the SFO plays a major function for aversive taste tolerance. FIG. 12A displays UMAP embedding of Ptger1, Ptger2 and Ptger4 log-normalized expression in the SFO demonstrating minimal expression of these three prostaglandin receptors. FIG. 12B displays data showing Ptger3^{Cre/Cre} mice did not show tolerance toward aversive taste under osmotic thirst (n=4-9 mice). As shown in FIG. 12C, knocking down Ptger3 in the SFO in the background of Trpm5−/− animals did not reduce tolerance toward aversive salt under osmotic thirst. The lick number of water and 500 mM NaCl in scramble-(grey, first bar each solution) and Ptger3-(red, second bar each solution) shRNA-injected Trpm5−/− animals are shown (n=5-6 mice). Data are expressed as mean±SEM, *p<0.05, **p<0.01.

FIG. 13A-FIG. 13I display non-limiting exemplary data related to functional characterization of the PGE2/Ptger3 axis for aversive taste tolerance. FIG. 13A shows measurement of inflammation-related factors in the circulation by ELISA. Progesterone and serotonin (5-HT) levels were measured from sated (grey), formalin-injected (blue), and sodium depleted (red) animals (n=7-8 mice). Shown in FIG. 13B is data related to control stimuli for fiber photometry recording from SFO^{Ptger3} neurons. These neurons showed no activation toward i.p. mannitol injection (2 M, 10 ml per kg BW; osmotic thirst), ghrelin (10 μg; hunger), or LiCl (0.15 M, 15 ml per kg BW; visceral malaise). Calcium dynamics and response amplitude (normalized ΔF/F) of SFO^{Ptger3} neurons are shown (n=5 mice). As shown in FIG. 13C, PGE2 activated the SFO in a Ptger3-dependent pattern. Representative images and quantification of Fos immunofluorescence signals after subcutaneous injection of PGE2 in Ptger3^{Cre/wt} (grey, first bar, n=4 mice) and Ptger3^{Cre/Cre} mice (red, second bar, n=4 mice) are shown. FIG. 13D displays data showing Ptger3 in the SFO is required for PGE2 responses. Representative images after subcutaneous injection of PGE2 in scramble-(top) and Ptger3-(bottom) shRNA-injected mice were shown. No Fos signal was found in the pre-LC. FIG. 13E displays data related to PGE2 induced tolerance in wild-type animals. Consumption of 500 mM NaCl was measured under sated and osmotic thirst after subcutaneous injection of vehicle (grey, first bar each condition) or PGE2 (red, second bar each condition, n=9 mice). FIG. 13F displays data related to PGE2-induced aversive taste tolerance in food-deprived, wild-type animals. Left, a diagram of the behavioral paradigm. Right, the number of licks toward 0.25 mM quinine with 500 mM glucose in food-deprived mice after vehicle (grey, first bar) or PGE2 injection (red, second bar, n=6). As shown in FIG. 13G, thirsty Ptger3^{Cre/Cre} mice consumed pure water after vehicle (grey, first bar) or PGE2 (red, second bar) subcutaneous injection (n=4 mice). FIG. 13H displays data showing thirsty Ptger3 KD mice consumed pure water after vehicle (grey) or PGE2 (red) subcutaneous injection (n=8-10 mice). As shown in FIG. 13I, Formalin-induced inflammation increased tolerance towards 500 mM NaCl during thirst (n=5-10). Water intake was unaffected by the same treatment. Data for Formalin-treated mice is the second bar for each solution. Data are expressed as mean±SEM, *p<0.05, **p<0.01, ***p<0.001. Scale bar, 100 m.

FIG. 14A-FIG. 14E display non-limiting exemplary data related to bimodal modulations of attractive and aversive taste signals by the brain. FIG. 14A displays a diagram of bimodal salt taste modulation. Low salt through the ENaC pathway and aversive high salt through bitter/sour pathways are modulated by two separate interoceptive circuits in the forebrain (SFO) and hindbrain (pre-LC) circuits. These circuits sense internal sodium balance and trigger bimodal modulation signals that affect the hedonic value of salt in a concentration- and internal-state-dependent manner. FIG. 14B displays representative taste responses from chorda tympani nerve under distinct internal states. As shown in FIG. 14C, 10, 30, 60, 120, 250, and 500 mM NaCl were applied to the tongue, and integrated nerve responses were quantified for each concentration (n=5 mice). FIG. 14D displays taste nerve recording with SFO^{Ptger3} photostimulation. Left, a diagram of chorda tympani nerve recording combined with optogenetics. Right, quantification of normalized responses to NaCl without (black), or with photostimulation (red) state (n=3 mice). ChR2 expression was validated by immunohistology and behavioral tolerance induction. In FIG. 14E, AAV-DIO-ChR2-EYFP was injected into SFO^{Ptger3} and the downstream projections were visualized. Strong projections were found in the following brain areas: MnPO, vBNST, paraventricular nucleus of the thalamus (PVT), paraventricular nucleus of the hypothalamus (PVN), and dorsomedial hypothalamus (DMH). Scale bar, 100 m.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods of reducing aversive taste tolerance in a subject in need thereof. In some embodiments, the method comprises: inhibiting a plurality of prostaglandin receptor type 3 (Ptger3)-positive neurons in the lamina terminalis (LT^{Ptger3}-neurons) of the subject, thereby reducing aversive taste tolerance in the subject.

Disclosed herein include methods of increasing aversive taste tolerance in a subject in need thereof. In some embodiments, the method comprises: stimulating a plurality of prostaglandin receptor type 3 (Ptger3)-positive neurons in the lamina terminalis (LT^{Ptger3}-neurons) of the subject, thereby increasing aversive taste tolerance in the subject.

Disclosed herein include methods of identifying a modulator of aversive taste tolerance. In some embodiments, the method comprises: (a) contacting a candidate compound with a plurality of prostaglandin receptor type 3 (Ptger3)-positive neurons in the lamina terminalis (LT^{Ptger3}-neurons) to determine an electrophysiological response in the LT^{Ptger3}-neurons; (b) identifying the candidate compound as a modulator of the LT^{Ptger3}-neurons if the electrophysiological response in the LT^{Ptger3}-neurons contacted with the candidate compound is altered as compared to the electrophysiological response in the LT^{Ptger3}-neurons prior to contacting with the candidate compound; (c) administering the identified modulator of the LT^{Ptger3}-neurons to a subject; (d) assessing the change in valence toward sodium and/or one or more aversive substances of the subject in response to the administration of the identified modulator of the LT^{Ptger3}-neurons; and (e) identifying the identified modulator of the LT^{Ptger3}-neurons as a modulator for aversive taste tolerance if the identified modulator of the LT^{Ptger3}-neurons changes the valence toward sodium and/or one or more aversive substances of the subject compared to a control.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “neurons” is used to refer to cells found in the nervous system that are specialized to receive, process, and transmit information as nerve signals. Neurons can include a central cell body or soma, and two types of projections: dendrites, by which, in general, the majority of neuronal signals are conveyed to the cell body; and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons).

As used herein, the term “modulating” a neuron refers to modulating the activity of a neuron. Modulating a neuron can be, for example, inhibiting the neuron or stimulating the neuron. The neuron can be, for example, photosensitive. In some embodiments, the neuron can be inhibited by electromagnetic radiation (e.g., light). In some embodiments, the neuron can be stimulated by electromagnetic radiation (e.g., light). The methods and compositions disclosed herein can be used to modulate the activity of a neuron for a selected duration, for example, 1 minute, 2 minutes, 3 minutes, 4 minutes, 5 minutes, 10 minutes, 20 minutes, 30 minutes, 40 minutes, 50 minutes, 100 minutes, 150 minutes, 200 minutes, or a range between any two of these values, or more. In some embodiments, the neuron is modulated (inhibited or stimulated) throughout the selected duration. In some embodiments, the neuron is modulated (inhibited or stimulated) with intervals of non-modulating break during the selected duration.

As used herein, the term “inhibiting” a neuron (for example, a prostaglandin receptor type 3 (Ptger3)-positive neuron as described herein (including an LT^{Ptger3}-neuron) has its customary and ordinary meaning as would be understood by one of skill in the art in view of the present disclosure. It refers to reducing the likelihood of, delaying the onset of, and/or preventing depolarization of the cell membrane of the neuron (which may also be referred to as the plasma membrane), and thus, reducing the likelihood of, delaying the onset of, and/or preventing the neuron from generating an action potential or firing. In some embodiments, an inhibited neuron may not induce an action potential or fire. For example, a neuron can be inhibited by inducing a net efflux of cations from the cytosol and/or by inhibiting, reducing the likelihood of, or preventing a net influx of cations into the cytosol. For example, a neuron can be inhibited by inducing, increasing the likelihood of, or stimulating a net influx of anions into the cytosol. In some embodiments, a net efflux of cations comprises cations leaving the cytosol through a channel or pump in the plasma membrane or the endoplasmic reticulum (ER). In some embodiments, a net influx of anions comprises anions entering the cytosol across the plasma membrane. Non-limiting examples of cations include protons (H), potassium (K+), calcium (Ca²⁺), and any combination thereof. A non-limiting example of anion is chloride (Cl). The level by which a neuron is inhibited can vary, depending on, for example, the inhibition mechanism. For example, a neuron can be inhibited when the likelihood of an action potential (compared to an untreated or unaltered neuron over a specified period of time, for example, 0.01, 0.1, 1, or 10 seconds) is reduced by at least, or by at least about, 50%, 60%, 70%, 80%, 85%, 90%, 95%, 99%, 99.5%, 99.9%, or more. In some embodiments, inhibiting a neuron silences that neuron.

As used herein, the terms “decrease,” “reduce,” “reduced,” “reduction,” “decrease,” and “inhibit” are used generally to refer to a decrease by an amount relative to a reference. The decreased amount relative to the reference can be, for example, a decrease by, by about, by at least, or by at least about, 20%, 25%, 30%, 35%, 40%, 45%, 50%, 55%, 60%, 65%, 70%, 75%, 80%, 85%, 90%, 95%, 98%, 99%, or a range between any two of these values, as compared to the reference.

As used herein, the terms “increase,” “stimulate,” “enhance” and “activate” are used to generally refer to an increase by an amount relative to a reference. The increased amount relative to the reference can be, for example, an increase by, by about, by at least, or by at least about, 20%, 25%, 30%, 35%, 40%, 45%, 50%, 55%, 60%, 65%, 70%, 75%, 80%, 85%, 90%, 95%, 98%, 99%, or a range between any two of these values, as compared to the reference. In some embodiment, the increase is, or is about, or is at least, or is at least about, a 2-fold, or at least about a 3-fold, or at least about a 4-fold, or at least about a 5-fold or at least about a 10-fold increase, or any increase between 2-fold and 10-fold or greater as compared to the reference.

As used herein, “stimulating” a neuron (e.g., a LT^{Ptger3}-neuron) has its customary and ordinary meaning as would be understood by one of skill in the art in view of the present disclosure. It refers to increasing the likelihood of, expediting the onset of, and/or inducing depolarization of the cell membrane of the neuron, and thus, increasing the likelihood of, expediting the onset of, and/or inducing an action potential in the neuron. For example, a neuron can be stimulated by a net efflux of anions from the cytosol, and/or a net influx of cations to the cytosol. In some embodiments, a stimulated neuron can be depolarized, inducing an action potential or firing of the neuron. Depolarization can be the result of a net influx of cations into the cytosol of the neuron. Cations can enter the cytosol though a channel in the plasma membrane and/or ER. The cations may comprise protons (FT), sodium (Na⁺) ions, calcium (Ca²⁺) ions, or a combination thereof. The level by which a neuron is stimulated can vary, depending on, for example, the stimulation mechanism. For example, a neuron can be stimulated when the likelihood of an action potential (compared to an unaltered neuron over a specified period of time, for example 0.01, 0.1, or 1 second) is increased by at least 50%, 60%, 70%, 80%, 85%, 90%, 95%, 99%, 99.5%, 99.9%, or more. In some embodiments, stimulating a neuron activates that neuron.

As used herein, the term “vector,” can refer to a vehicle for carrying or transferring a nucleic acid. Non-limiting examples of vectors include viral vectors (for example, adenovirus vectors, adeno-associated virus (AAV) vectors, retrovirus vectors, lentiviral vectors, herpes virus vectors, phages, and proxvirus vectors); non-viral vectors such as liposomes, naked DNA, plasmids, cosmids; and the like. The term “AAV” or “adeno-associated virus” refers to a Dependoparvovirus within the Parvoviridae genus of viruses.

As used herein, the term “construct,” refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil). Unless specified otherwise, the left-hand end of any single-stranded polynucleotide sequence discussed herein is the 5′ end; the left-hand direction of double-stranded polynucleotide sequences is referred to as the 5′ direction.

As used herein, the term “subject” refers to an animal that is the object of treatment, observation or experiment. “Animal” includes cold- and warm-blooded vertebrates (e.g., mammals) and invertebrates (e.g., fish, shellfish and reptiles). “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees, apes, and humans. In some embodiments, the subject is a human. However, in some embodiments, the subject is not a human.

As used herein, the term “agonist” refers to any molecule or compound that fully or partially activates, stimulates, enhances, or promotes one or more of the biological properties of a biological entity, for example a protein, a nucleic acid, a cell (e.g., a neuron), an organ, or an organism. Agonists can include, but are not limited to, small organic and inorganic molecules, nucleic acids, peptides, peptide mimetics and antibodies.

As used herein, the term “effective amount” refers to an amount sufficient to effect beneficial or desirable biological and/or clinical results. For example, an “effective amount” of a modulator of aversive taste tolerance is an amount of the modulator, alone or in combination with one or more other therapies and/or agents, sufficient to cause an alteration in the aversive taste tolerance of a subject in need thereof.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. The physiologically acceptable carrier can be an aqueous pH buffered solution such as phosphate buffer or citrate buffer, and can also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

### Sodium and Aversive Taste Tolerance

Described herein is a neural substrate in the circumventricular organs which is activated by the elevated prostaglandin E2 (PGE2) level during sodium depletion. The PGE2 receptor type 3 (Ptger3)-expressing neurons in the subfornical organ (SFO) and vascular organ of lamina terminalis (OVLT) are the main targeted manipulation sites. An independent neural pathway encoding the aversive taste tolerance to allow a higher consumption of salt during sodium depletion was discovered as described herein. It was also found that depletion of sodium induced an increased PGE2 production and shifted the body to a more pro-inflammatory state. The elevated PGE2 activated the PGE2 neurons in the lamina terminalis (LT) and this interaction is sufficient and necessary for the aversive taste tolerance.

In some embodiments, the Ptger3-PGE2 axis may be used to modulate sodium intake in daily life and clinical setting. The action site (LT) is a brain region without the blood brain barrier (BBB). The lack of the BBB allows efficient drug/small chemical delivery in further studies. Some embodiments of the methods and compositions described herein include: daily supplements for modulating salt consumption, drugs for salt-sensitive hypertension patients, and food taste enhancers. Compositions and methods for modulation of aversive taste tolerance can include usage of plant extract or synthesized small molecules as Ptger3 antagonists to reduce the tolerance to high sodium and reduce salt consumption accordingly.

Disclosed herein include methods for modulating aversive taste tolerance in a subject. Disclosed herein include methods of reducing aversive taste tolerance in a subject in need thereof. In some embodiments, the method comprises: inhibiting a plurality of prostaglandin receptor type 3 (Ptger3)-positive neurons in the lamina terminalis (LT^{Ptger3}-neurons) of the subject, thereby reducing aversive taste tolerance in the subject. Disclosed herein include methods of increasing aversive taste tolerance in a subject in need thereof. In some embodiments, the method comprises: stimulating a plurality of prostaglandin receptor type 3 (Ptger3)-positive neurons in the lamina terminalis (LT^{Ptger3}-neurons) of the subject, thereby increasing aversive taste tolerance in the subject.

For example, increased aversive taste tolerance can result in, e.g., increased ingestion of high salt (e.g., 300 mM or greater) and/or one or more aversive substances (e.g., bitter or sour-tasting substances). In another example, decreased aversive taste tolerance can result in, e.g., decreased ingestion of high salt (e.g., 300 mM or greater) and/or one or more aversive substances (e.g., bitter or sour-tasting substances) by a subject.

The lamina terminalis is a forebrain structure lying on the anterior wall of the third ventricle that plays a key role integrating signals for the regulation of numerous aspects of homeostasis including not only cardiovascular function but also immune, reproductive, and metabolic functions. It is comprised of two sensory circumventricular organs (CVOs), the SFO and OVLT, as well as the hypothalamic MnPO. The sensory CVOs, the SFO, and OVLT are highly vascularized and lack a blood-brain barrier, positioning the neurons located there into a privileged position of direct contact with signals such as hormones, cytokines, and other constituents of the circulation. While the parenchyma of the sensory CVOs is accessible to the circulation, it is effectively protected from the cerebrospinal fluid by specialized tanycytes on the ependymal surface which are joined by tight junctions.

The SFO is a translucent ovoid structure protruding into the third ventricle between the columns of the fornix, dorsal to the anterior commissure, on the dorsal aspect of the hippocampal commissure. The OVLT is found in the ventral aspect of the anterior wall of the third ventricle, just anterior and dorsal to the optic chiasm. Early studies of the OVLT often refer to it as the supraoptic crest, due to the anatomical shape where its bulbous base protrudes into the optic recess of the third ventricle, but the main body narrows dorsally, giving it an elongated crest-like shape.

Located immediately dorsal to the OVLT, and ventral to the SFO, is the MnPO. Unlike the SFO and OVLT, the MnPO has an intact blood-brain barrier and does not stain when animals are injected with intravital dyes or horse-radish peroxidase. The MnPO is divided into two key areas, the dorsal MnPO that is the tissue found adjacent to the anterior commissure and ventral to the SFO, while the ventral aspect extends ventrally from the anterior commissure to the dorsal cap of the OVLT. Based on having an intact blood-brain barrier, and the number of afferent connections from visceral sensory areas including nucleus of the solitary tract and ventrolateral medulla SFO and OVLT, it stands that the role of the MnPO is as an integrating center for the lamina terminalis.

The subjects that the methods and compositions described herein are applicable to include, but are not limited to, human subjects and non-human subjects (e.g., non-human mammals). The age and gender of the subject can vary. For example, the subject can an elderly subject, a juvenile, an infant, or an adult. As used herein, an “elderly” subject refers to a human that is at least 50 years old, for example at least 50, 55, 60, 65, 70, 75, 80, 85, 90, or a range between any two of these values, years old. The subject can be, or be about, one-day old, one-month old, six-month old, one-year old, two-year old, five-year old, ten-year old, twenty-year old, thirty-year old, forty-year old, fifty-year old, or a range between any two of these values. Subjects that are in need of the methods and compositions described herein for reducing sodium intake include a subject suffering from, or is at a risk of developing, a kidney disorder, kidney damage, a cardiovascular disease, high blood pressure, overweight, edema, left ventricular hypertrophy, stroke, or a combination thereof. The kidney disorder can be, for example, a chronic kidney disease or kidney failure. It is contemplated that a subject is at a risk of developing heart- and kidney-related diseases can benefit from reducing aversive taste tolerance. It is also contemplated that a subject suffering from heart- and kidney-related diseases can benefit from reducing aversive taste tolerance. By way of example, in some embodiments, aversive taste tolerance in a subject can be reduced compared to the aversive taste tolerance of the subject before application of the methods/compositions described herein for reducing aversive taste tolerance. In some embodiments, ingestion of sodium and/or one or more aversive substances in a subject can be reduced to a level of ingestion of sodium and/or one or more aversive substances recommended as healthy for the subject. In some embodiments, a subject is at a risk of developing high blood pressure or is suffering from high blood pressure. In some embodiments, the subject is at a risk of developing a cardiovascular disease or is suffering from a cardiovascular disease.

### Conditional Ion Modulators

As used herein, “conditional ion modulators” refers to chemogenetic receptors and optogenetic actuators. In some embodiments, the conditional ion modulator comprises, or is, a chemogenetic receptor. In some embodiments, the conditional ion modulator comprises, or is, an optogenetic actuator. A “conditional ion modulator nucleic acid” is used herein to refer to a nucleic acid that encodes a conditional ion modulator (e.g., an optogenetic receptor or chemogenetic receptor).

Chemogenetic receptors can be used in the methods and compositions disclosed herein to modulate (e.g., inhibit or enhance) aversive taste tolerance, and the methods and compositions disclosed herein for identifying modulators for aversive taste tolerance. As used herein, “chemogenetic receptor” refers to a receptor that can be expressed in a cell and modulates movement of ions in or out of the cell when a condition is present, for example binding of an agonist such as a small molecule such as clozapine N-oxide (CNO). For example, the chemogenetic receptor can comprise a G protein coupled receptor and can conditionally induce signaling in the cell that expresses the receptor. Examples of chemogenetic receptors include, but are not limited to, Designer Receptors Exclusively Activated by Designer Drugs (DREADDs). In some embodiment, the DREADD may encode a receptor such as a G protein coupled receptor configured to depolarize or activate a neuron (e.g., an LT^{Ptger3}-neuron) Non-limiting exemplary chemogenetic receptors are describe in Roth (2016), “DREADDs for Neuroscientists” Neuron. 89: 683-694, which is incorporated by reference in its entirety herein. In some embodiments, the chemogenetic receptor comprise an ion channel or ion pump, or be in signal transduction communication with an ion channel or ion pump. As used herein, a “chemogenetic receptor nucleic acid” refers to a nucleic acid that encodes a chemogenetic receptor. In some embodiments, the optogenetic actuator comprises or is hM3 DREADD or hM4Di. hM3 DREADD comprises a modified human M3 muscarinic receptor and is activated by the agonist CNO. The CNO can be administered to a subject, for example systemically or directly to the CNS, and can thus bind to the chemogenetic receptor (such as hM3 DREADD). Binding of CNO to hM3 DREADD induces Gq G-protein coupled signaling, which induces the release of intracellular calcium in neurons, enhancing neuron activation. CNO can be administered to a subject nasally, transcranially, intracranially, orally, intravenously, subcutaneously, transdermally, intreperitoneally, nasally, or any combination thereof.

Optogenetic actuators can be used in the methods and compositions disclosed herein to modulate (e.g., inhibit or enhance aversive taste tolerance, and the methods and compositions disclosed herein for identifying modulators for aversive taste tolerance. As used herein, “optogenetic actuator” refers to an ion transporter that can be expressed in a cell, and directly or indirectly transport ions (into or out of the cytosol) when a condition is present, for example upon stimulation with electromagnetic radiation. An optogenetic actuator can comprise, or can be, a passive transporter (such as an ion channel) and/or an active transporter (such as an ion pump). For example, the optogenetic actuator can comprise an ion channel or an ion pump, and can conditionally permit or prevent the passage of ions through the ion channel. In some embodiments, the optogenetic actuator comprises, or is, a channelrhodopsin, halorhodopsin and/or archaeorhodopsin. Non-limiting exemplary optogenetic actuators are described in Lin (2011) “A User's Guide to Channelrhodopsin Variants: Features, Limitations and Future Developments” Exp. Physiol. 96: 19-25, which is incorporated by reference in its entirety herein. As used herein, an “optogenetic actuator nucleic acid” refers to a nucleic acid that encodes an optogenetic actuator.

In some embodiments, the conditional ion modulator comprises an optogenetic actuator such as a channelrhodopsin (e.g., ChR2 or VChR1). Channelrhodopsin comprises an ion channel, the opening of which is stimulated by electromagnetic radiation of a suitable wavelength. For example, ChR2 is stimulated by light in the blue spectrum (e.g., about 450 nm to about 470 nm) and VChR1 is stimulated by light in the green spectrum (e.g., about 550 nm to about 570 nm). In some embodiments of the methods and compositions disclosed herein, the conditional ion modulator comprises an optogenetic receptor, and is stimulated by electromagnetic radiation, thus inducing opening of an ion channel and a change in polarity of the neuron that expresses the conditional ion modulator.

In some embodiments, the conditional ion modulator is configured to inhibit stimulation of a neuron or inhibit a neuron, for example by inducing a net efflux of cations from a cytosol and/or induce a net influx of anions to the cytosol. Such conditional ion modulators are referred to herein as “inhibitory conditional ion modulators.” Non-limiting examples of inhibitory conditional ion modulators include hM4Di, halorhodopsin, and archaeorhodopsin. hM4Di receptors can inhibit neurons upon stimulation with their agonist, for example CNO. The hM4Di receptor comprises a modified form of the human M4 muscarinic (hM4) receptor. The hM4Di receptor can be activated by CNO, engaging the Gi signaling pathway. Gi signaling in neurons results in the opening of potassium channels and an influx of potassium ions, decreasing the capacity of the neuron to depolarize. Neurons expressing hM4Di that are treated with CNO can have decreased firing rates. Halorhodopsin comprises a transmembrane chloride channel, which can move chloride channels into the cell in response to electromagnetic radiation in the green to yellow spectrum of visible light. Archaeorhodopsin comprises a transmembrane proton pump, which can pump proteins out of the cell in response to light, thereby hyperpolarizing the neuron, and inhibiting an action potential by the neuron. In some embodiments of the methods and compositions disclosed herein, a conditional ion modulator inhibits a neuron (e.g., a LT^{Ptger3}-neuron).

In some embodiments, the conditional ion modulator is configured to stimulate a neuron, for example by inducing a net influx of cations into a cytosol and/or induce a net efflux of anions from the cytosol. Such conditional ion modulators are referred to herein as “stimulatory conditional ion modulators.” Examples or such stimulatory conditional ion modulators include hM3 DREADD and/or channelrhodopsin. In some embodiments, for example methods and compositions in which a conditional ion modulator inhibits a neuron, the conditional ion modulator comprises hM3 DREADD and/or channelrhodopsin.

### Compositions for Nucleic Acid Delivery to a Subject and Methods of Administration

Various systems and methods are known in the art for delivering nucleic acid molecules into a cell, a tissue, an organ, and/or a subject. The delivery can be, for example, target-specific, tissue-specific, cell type specific, organ specific, nonspecific, and/or systematic. In some embodiments, the nucleic acid molecule comprises a coding sequence for one or more proteins, and the delivery is used for expressing the one or more proteins encoded by the nucleic acid molecule in the target cell, tissue, organ, and/or subject.

Disclosed include nucleic acids (e.g., an expression vector) in a composition (e.g., a pharmaceutical composition). The nucleic acids can, for example, comprise a coding sequence for a conditional ion modulator, wherein the conditional ion modulator is expressed and/or activated in response to a stimulus or agonist. In some embodiments, activation of the conditional ion modulator can inhibit (e.g., specifically and/or selectively inhibit) a neuron population, for example LT^{Ptger3}-neurons. As another example, the nucleic acids can comprise a coding sequence for an inhibitory conditional ion modulator, wherein the inhibitory conditional ion modulator is expressed and/or activated in response to a stimulus or agonist. In some embodiments, expression of the inhibitory conditional ion modulator can stimulate (e.g., specifically and/or selectively stimulate) a neuron population, for example LT^{Ptger3}-neurons.

Many different viral and non-viral vectors and methods of their delivery, for use in gene delivery (including gene therapy), are known, including adenovirus vectors, adeno-associated virus (AAV) vectors, retrovirus vectors, lentiviral vectors, herpes virus vectors, liposomes, proxviruses, naked DNA administration, plasmids, cosmids, phages, encapsulated cell technology, and the like. A detailed review of possible techniques for transforming genes into desired cells of the eye is taught by Wright (Br J Ophthalmol, 1997; 81: 620-622). The vectors (e.g., an AAV vector) can be used to deliver the coding sequence for the stimulatory conditional ion modulator or the inhibitory conditional ion modulator to a subject in need thereof. The expression of the stimulatory conditional ion modulator or the inhibitory conditional ion modulator from the nucleic acid (e.g., the expression vector) can be controlled by a transcription regulatory element, for example for example, a cell specific promoter to allow expression occurred only in a specific cell type (e.g., neurons), or a promoter selected from Human elongation factor-1 alpha (EF-1 alpha), Human cytomegalovirus promoter (CMV), and CAG promoter. Titers of the viral vector to be administered will vary depending, for example, on the particular viral vector, the mode of administration, the treatment goal, the individual, and the cell type(s) being targeted, and can be determined by methods standard in the art.

Adeno-associated virus (AAV) is a replication-deficient parvovirus, the single-stranded DNA genome of which is about 4.7 kb in length including 145 nucleotides inverted terminal repeat (ITRs). The ITRs play a role in integration of the AAV DNA into the host cell genome. When AAV infects a host cell, the viral genome integrates into the host's chromosome resulting in latent infection of the cell. In a natural system, a helper virus (for example, adenovirus or herpesvirus) provides genes that allow for production of AAV virus in the infected cell. In the case of adenovirus, genes ElA, E1B, E2A, E4 and VA provide helper functions. Upon infection with a helper virus, the AAV provirus is rescued and amplified, and both AAV and adenovirus are produced. In the instances of recombinant AAV vectors having no Rep and/or Cap genes, the AAV can be non-integrating.

AAV vectors that comprise coding sequences of the stimulatory conditional ion modulator or the inhibitory conditional ion modulator are provided. The AAV vector can include a 5′ inverted terminal repeat (ITR) of AAV, a 3′ AAV ITR, a promoter, and a restriction site downstream of the promoter to allow insertion of a polynucleotide encoding the stimulatory conditional ion modulator or the inhibitory conditional ion modulator, wherein the promoter and the restriction site are located downstream of the 5′ AAV ITR and upstream of the 3′ AAV ITR. In some embodiments, the AAV vector includes a posttranscriptional regulatory element downstream of the restriction site and upstream of the 3′ AAV ITR.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

In some embodiments, the viral vectors can include additional sequences that make the vectors suitable for replication and integration in eukaryotes. In some embodiments, the viral vectors disclosed herein can include a shuttle element that makes the vectors suitable for replication and integration in both prokaryotes and eukaryotes. In some embodiments, the viral vectors can include additional transcription and translation initiation sequences, such as promoters and enhancers; and additional transcription and translation terminators, such as polyadenylation signals. Various regulatory elements that can be included in an AAV vector have been described in detail in US2012/0232133 which is hereby incorporated by reference in its entirety.

The pharmaceutical composition can comprise one or more nucleic acids disclosed herein and one or more pharmaceutically acceptable carriers. The composition can also comprise additional ingredients such as diluents, stabilizers, excipients, and adjuvants. As used herein, “pharmaceutically acceptable” carriers, excipients, diluents, adjuvants, or stabilizers are the ones nontoxic to the cell or subject being exposed thereto (preferably inert) at the dosages and concentrations employed or that have an acceptable level of toxicity as determined by the skilled practitioners.

The carriers, diluents and adjuvants can include buffers such as phosphate, citrate, or other organic acids; antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution.

As will be readily apparent to one of skill in the art, the useful in vivo dosage of the recombinant virus to be administered and the particular mode of administration will vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

Although the exact dosage will be determined on a drug-by-drug basis, in most cases, some generalizations regarding the dosage can be made. In some embodiments, the viral vector for delivery a nucleic acid to a subject (e.g., systematic delivery, or delivery to the brain tissue of the subject) can be administered, for example via injection, to a subject at a dose of between 1×10¹⁰ genome copies (GC) of the recombinant virus per kg of the subject and 2×10¹⁴ GC per kg, for example between 5×10¹¹ GC/kg and 5×10¹² GC/kg. In some embodiments, the dose of the viral vector (e.g., AAV vectors) administered to the subject is no more than 2×10¹⁴ GC per kg. In some embodiments, the dose of the viral vector administered to the subject is no more than 5×10¹² GC per kg. In some embodiments, the dose of the viral vector administered to the subject is no more than 5×10ⁿ GC per kg.

The nucleic acid molecule, for example, a vector (e.g., a viral vector)) comprising a coding sequence of a stimulatory conditional ion modulator or an inhibitory conditional ion modulator can be administered to a subject (e.g., a human) in need thereof. The route of the administration is not particularly limited. For example, a therapeutically effective amount of the nucleic acid molecule can be administered to the subject by via routes standard in the art. Non-limiting examples of the route include intravitreal, intravenous, intraocular, or subretinal administration, depending on the retinal layer being targeted. In some embodiments, the nucleic acid molecule is administered to the subject by systematic transduction. In some embodiments, the nucleic acid molecule is administered to the subject by intravenous injection. In some embodiments, the nucleic acid molecule is administered to the subject by subretinal injection. In some embodiments, the administration of the nucleic acid molecule targeting of retinal pigment epithelium—the most distal layer from the vitreal space. In some embodiments, the delivery of the nucleic acid molecule is targeted to retinal ganglion cells, bipolar cells, or both. The ganglion cells are, in some embodiments, accessible to intravitreal injection as disclosed herein. Intravitreal and/or subretinal injection can be used, in some embodiments to target the bipolar cells, for example in circumstances in which the photoreceptor cell layer is absent due to degeneration.

Actual administration of the expression vectors for the stimulatory conditional ion modulator or the inhibitory conditional ion modulator can be accomplished by using any physical method that will transport the vectors (e.g., viral vectors) into the target tissue(s) (e.g., brain) of the subject. In some embodiments, the vectors can be administered systematically, e.g., by intravenous injection. Pharmaceutical compositions can be prepared, for example, as injectable formulations. The recombinant virus to be used can be utilized in liquid or freeze-dried form (in combination with one or more suitable preservatives and/or protective agents to protect the virus during the freeze-drying process). For gene therapy (e.g., of neuronal disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the viral vector (e.g., AAV vector) have been established for at least some condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the expression vector (e.g., AAV vector) can be administered to a subject at various points of time. For example, the expression vector can be administered to the subject prior to, during, or after the subject has developed a disease or disorder. The expression vector can also be administered to the subject prior to, during, or after the occurrence of a disease or disorder (e.g., neuronal disorders, ocular disorders, or a combination thereof). In some embodiments, the expression vector is administered to the subject during remission of the disease or disorder. In some embodiments, the expression vector is administered prior to the onset of the disease or disorder in the subject. In some embodiments, the expression vector is administered to a subject at a risk of developing the disease or disorder.

The dosing frequency of the expression vector (e.g., viral vector) can vary. For example, the viral vector can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. In some embodiments, the viral vector is administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

### Methods of Reducing Aversive Taste Tolerance

Disclosed herein include methods of reducing aversive taste tolerance in a subject in need thereof. In some embodiments, the method comprises: inhibiting a plurality of prostaglandin receptor type 3 (Ptger3)-positive neurons in the lamina terminalis (LT^{Ptger3}-neurons) of the subject, thereby reducing aversive taste tolerance in the subject.

In some embodiments, the method comprises inhibiting depolarization of the cell membranes of the plurality LT^{Ptger3}-neurons. Thus, stimulation of the plurality of LT^{Ptger3}-neurons can be inhibited, thus inhibiting intake of sodium and/or one or more aversive substances in the subject. In some embodiments, the method comprises at least one of inhibiting cation influx into a cytosol of the plurality of LT^{Ptger3}-neurons, inducing anion influx into the cytosol of the plurality of LT^{Ptger3}-neurons, and inducing cation efflux from the cytosol of the plurality of LT^{Ptger3}-neurons. In some embodiments, the method comprises administering a vector encoding a conditional ion modulator to the subject as described herein.

Reducing aversive taste tolerance in the subject can comprise reducing ingestion of sodium, one or more aversive substances, or a combination thereof by the subject. The sodium can be at a concentration of less than 120 mM (e.g., a low salt concentration). For example, the concentration of sodium can be 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM or a number or a range between any two of these values. The sodium can be at a concentration of greater than 300 mM (e.g., high salt concentration). For example, the concentration of sodium can be 300 mM, 400 mM, 500 mM, 600 mM, 700 mM, 800 mM, 900 mM, 1000 mM, or a number or a range between any two of these values, or greater.

In some embodiments, reducing aversive taste tolerance in the subject comprises reducing ingestion of sodium and one or more aversive substances by the subject, and wherein the sodium is at a concentration of less than 120 mM (1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM or a number or a range between any two of these values).

The concentration of any of the one or more aversive substances can vary. The one or more aversive substances can have a concentration of about 0.001 mM, 0.01 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM, 150 mM, 300 mM, 400 mM, 500 mM, 600 mM, 700 mM, 800 mM, 900 mM, 1000 mM, or a number or a range between any two of these values, or greater.

The one or more aversive substances can comprise one or more aversive minerals, one or more bitter-tasting substances, one or more sour-tasting substances, or any combination thereof. The one or more aversive minerals can comprise potassium chloride (KCl), calcium chloride (CaCl₂), and/or magnesium chloride (MgCl₂). The one or more bitter-tasting substances can comprise polyphenols, alkaloids, terpenoids, saponins, amino acids, bitter peptides, or any combination thereof. The one or more bitter-tasting substances can comprise quinine. The one or more sour-tasting substances can comprise lactic acid, citric acid, malic acid, acetic acid, or any combination thereof.

The method comprises, in some embodiments, determining sodium intake in the subject before inhibiting the plurality of LT^{Ptger3}-neurons of the subject, determining sodium intake in the subject after inhibiting the plurality of LT^{Ptger3}-neurons of the subject, or both. The extent to which the aversive taste tolerance (e.g., ingestion of sodium and/or one or more aversive substances) is reduced in the subject can vary. For example, ingestion of sodium and/or one or more aversive substances can be reduced by, by about, by at least, or by at least about, 10%, 20%, 30%, 40%, 50%, 60%, 70%, 80%, 90%, 95%, 98%, or a range between any two of these values, as compared to the subject before application of the method. The aversive taste tolerance of the subject can be reduced by, by about, by at least, or by at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) in the subject, e.g., as assessed by ingestion of the one or more aversive substances and/or sodium by the subject.

The LT^{Ptger3}-neurons can be in the subfornical organ of the LT (SFO^{Ptger3}-neurons), the vascular organ of the LT (OVLT^{Ptger3}-neurons), or both. The SFO^{Ptger3}-neurons can be GLUT1-expressing excitatory neurons. In some embodiments, SFO^{Ptger3}-neurons do not express GLUT2, GLUT3, GLUT4, and/or GLUT5.

Various inhibition methods/techniques can be used to inhibit LT^{Ptger3}-neurons. Inhibiting the plurality of LT^{Ptger3}-neurons of the subject can comprise administration of a Ptger3-inhibitor to the subject. The Ptger3-inhibitor can inhibit Ptger3 expression, activity, or both. The Ptger3-inhibitor can inhibit activity of a prostaglandin. The prostaglandin can be Prostaglandin E2 (PGE2). The Ptger3-inhibitor can be a small molecule, an antibody, or a nucleic acid. The nucleic acid can be an anti-sense RNA. The anti-sense RNA can comprise a microRNA (miRNA), a precursor microRNA (pre-miRNA), a small interfering RNA (siRNA), a short-hairpin RNA (shRNA), precursors thereof, derivatives thereof, or a combination thereof. The shRNA can comprise the sequence of SEQ ID NO: 1 (CCGGGCCGCTATTGATAATGATGTTCTCGAGAACATCATTATCAATAGCGGCTTTTT G; for RNA Thymine (T) is Uracil (U)). The shRNA can comprise a sequence at least about 85%, 86%, 87%, 88%, 89%, 90%, 91%, 92%, 93%, 94%, 95%, 96%, 97%, 98%, 99%, 100%, or a number or a range between any two of these values, identical to SEQ ID NO: 1.

A Ptger-3 inhibitor comprising a nucleic can be delivered to a subject via any method known in the art. For example, the nucleic acid can be complexed or associated with one or more lipids or lipid-based carriers, thereby forming liposomes, lipid nanoparticles (LNPs), lipoplexes, and/or nanoliposomes, e.g., encapsulating the nucleic acid composition. In some embodiments, the nucleic acid comprises, or further comprises, one or more vectors. At least one of the one or more vectors can be a viral vector, a plasmid, a transposable element, a naked DNA vector, a lipid nanoparticle (LNP), or any combination thereof.

The nucleic acid can comprise at least one regulatory element. The nucleic acid can comprise a vector. In some embodiments, the vector can comprise a adenovirus vector, an adeno-associated virus vector, an Epstein-Barr virus vector, a Herpes virus vector, an attenuated HIV vector, a retroviral vector, a vaccinia virus vector, or any combination thereof. In some embodiments, the vector can comprise an RNA viral vector. In some embodiments, the vector can be derived from one or more negative-strand RNA viruses of the order Mononegavirales. In some embodiments, the vector can be a rabies viral vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Retroviral vectors can be “defective”, i.e. unable to produce viral proteins required for productive infection. Replication of the vector can require growth in the packaging cell line. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., a synthetic protein circuit component) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

In some embodiments, the vectors can include a regulatory sequence that allows, for example, the translation of multiple proteins from a single mRNA. Non-limiting examples of such regulatory sequences include internal ribosome entry site (TRES) and 2A self-processing sequence. In some embodiments, the 2A sequence is a 2A peptide site from foot-and-mouth disease virus (F2A sequence). In some embodiments, the F2A sequence has a standard furin cleavage site. In some embodiments, the vector can also comprise regulatory control elements known to one of skill in the art to influence the expression of the RNA and/or protein products encoded by the polynucleotide within desired cells of the subject. In some embodiments, functionally, expression of the polynucleotide is at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of said intron. Another example of a regulatory element is a transcription termination signal and/or a polyadenylation sequences.

Expression control elements and promoters include those active in a particular tissue or cell type, referred to herein as a “tissue-specific expression control elements/promoters.” Tissue-specific expression control elements are typically active in specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

In some embodiments, the nucleic acid comprises a promoter operably linked to the polynucleotide encoding a payload. In some embodiments, the promoter is capable of inducing the transcription of the polynucleotide. In some embodiments, the heterologous nucleic acid comprises one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). In some embodiments, the silencer effector comprises a microRNA (miRNA), a precursor microRNA (pre-miRNA), a small interfering RNA (siRNA), a short-hairpin RNA (shRNA), precursors thereof, derivatives thereof, or a combination thereof. In some embodiments, said silencer effector is capable of binding the one or more silencer effector binding sequences, thereby reducing the stability of the payload transcript and/or reducing the translation of the payload transcript. In some embodiments, the polynucleotide further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the promoter comprises a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken R-actin (CBA) promoter, a CAG promoter, a CBH promoter, or any combination thereof. In some embodiments, the promoter is an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter. In some embodiments, the promoter comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MIC) promoter, or a cardiac Troponin T (cTnT) promoter. In some embodiments, the tissue specific promoter is a neuron-specific promoter. In some embodiments, the neuron-specific promoter comprises a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. In some embodiments, the tissue specific promoter is a muscle-specific promoter. In some embodiments, the muscle-specific promoter comprises a creatine kinase (MCK) promoter. In some embodiments, the promoter comprises an intronic sequence. In some embodiments, the promoter comprises a bidirectional promoter and/or an enhancer. In some embodiments, the enhancer is a CMV enhancer. In some embodiments, one or more cells of a subject comprise an endogenous version of the payload, and wherein the promoter comprises or is derived from the promoter of the endogenous version. In some embodiments, one or more cells of a subject comprise an endogenous version of the payload, and wherein the payload is not truncated relative to the endogenous version.

Expression control elements also can confer expression in a manner that is regulatable, that is, a signal or stimuli increases or decreases expression of the operably linked polynucleotide. A regulatable element that increases expression of the operably linked polynucleotide m response to a signal or stimuli is also referred to as an “inducible element” (that is, it is induced by a signal). Particular examples include, but are not limited to, a hormone (for example, steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The nucleic acid can comprise RNA or DNA. In some embodiments, the RNA or DNA sequence comprises one or more non-naturally occurring nucleotide or nucleotide analogs such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides.

The Ptger3-inhibitor can be an anti-inflammatory agent. The anti-inflammatory agent can be a non-steroidal anti-inflammatory drug (NSAID). The NSAID can comprise ibuprofen, dexibuprofen, fenoprofen, flurbiprofen, ketoprofen, oxaprozin, naproxen, dexketoprofen, loxoprofen, aspirin, salicylic acid, diflunisal, salsalate, indomethacin, tolmetin, sulindac, etodolac, ketorolac, diclofenac, aceclofenac, bromfenac, nabumetone, piroxicam, meloxicam, tenoxicam, droxicam, lornoxicam, isoxicam, phenylbutazone, mefenamic acid, meclofenamic acid, flufenamic acid, tolfenamic acid, celecoxib, rofecoxib, valdecoxib, parecoxib, lumiracoxib, etoricoxib, firocoxib, nimesulide, clonixin, licofelone, H-harpagide, or any combination thereof. The Ptger3-inhibitor can be administered to the subject orally or intravenously.

In some embodiments, the inhibition can comprise optogenetic inhibition, chemogenetic inhibition, or both. In some embodiments, inhibiting the plurality of LT^{Ptger3}-neurons of the subject comprises inhibiting the plurality of LT^{Ptger3}-neurons by a conditional ion modulator. For example, inhibiting the plurality of LT^{Ptger3}-neurons can comprise: administering a nucleic acid encoding a conditional ion modulator to the subject, wherein the conditional ion modulator is activated in response to a stimulus or agonist; and applying an agonist or stimulus of the conditional ion modulator to the subject, causing the activation of the conditional ion modulator, thereby inhibiting the LT^{Ptger3}-neurons. In some embodiments, the conditional ion modulator comprises a chloride conducting channelrhodopsin (ChloC) or a halorhodopsin (HR), and the stimulus comprises an optical stimulus. The conditional ion modulator can comprise a designer receptor exclusively activated by designer drug (DREADD), and the agonist can be clozapine-N-oxide (CNO). In some embodiments, the nucleic acid is administered to the subject in an adeno-associated viral (AAV) vector.

The duration in which the plurality of LT^{Ptger3}-neurons is inhibited can vary. For example, a duration can be, 1 minute, 2 minutes, 3 minutes, 4 minutes, 5 minutes, 10 minutes, 20 minutes, 30 minutes, 40 minutes, 50 minutes, 100 minutes, 150 minutes, 200 minutes, or a range between any two of these values, or more. In some embodiments, the neuron is inhibited throughout the selected duration. In some embodiments, the neuron is inhibited with intervals of non-modulating break during the selected duration. In some embodiments, inhibiting the plurality of LT^{Ptger3}-neurons lasts for at least five minutes in total.

In some embodiments, the method comprises identifying the subject as in need of reducing aversive taste tolerance. For example, the subject can be identified as suffering from or at a risk of developing a kidney disorder, kidney damage, a cardiovascular disease, high blood pressure, overweight, edema, left ventricular hypertrophy, stroke, or a combination thereof. The subject, in some embodiments, is at the risk of developing, or is suffering from, a kidney disorder, kidney damage, a cardiovascular disease, high blood pressure, overweight, edema, left ventricular hypertrophy, stroke, or a combination thereof. In some embodiments, the subject is at the risk of developing, or is suffering from, a chronic kidney disease or kidney failure. In some embodiments, the subject is a human or a non-human mammal. In some embodiments, the subject is an elderly subject. In some embodiments, the method of reducing aversive taste tolerance is a method of ameliorating, inhibiting, delaying the onset of, reducing the severity of, or preventing a kidney disorder, kidney damage, a cardiovascular disease, high blood pressure, overweight, edema, left ventricular hypertrophy, stroke, or a combination thereof.

In some embodiments, the cations comprise protons, sodium cations, calcium cations, or a combination thereof. It will be appreciated that since the cytosol of a neuron (such as a LT^{Ptger3}-neuron) comprises cations, a net efflux of cations into the cytosol refers to a decrease in the quantity of cations in the cytosol compared to prior to the efflux. In some embodiments, the quantity of cations in the cytosol is decreased by at least 1%, for example at least 1%, 2%, 3%, 4%, 5%, 6%, 7%, 8%, 9% %, %15%, 20%, 25%, 30%, 35%, 40%, 45%, 50%, 55%, 60%, 65%, 70%, 75%, 80%, 90%, 95%, 96%, 97%, 98%, 99%, or 99.9%, including ranges between any two of the listed values compared to prior to the efflux. In some embodiments, the net efflux of cations is effective to prevent an action potential in the neuron. In some embodiments, anions (such as those that exhibit a net influx into the cytosol of a neuron) comprise chloride anions (Cl). Similarly, it will be appreciated that since the cytosol of a neuron (such as a LT^{Ptger3}-neuron) comprises anions, a net influx of anions from the cytosol refers to an increase in the quantity of anions in the cytosol compared to prior to the efflux. In some embodiments, the quantity of anions in the cytosol is increased by at least 1%, for example at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 90%, 95%, 100%, 150%, 200%, 300%, 400%, or 500%, including ranges between any two of these values compared to prior to the efflux. In some embodiments, the net influx of anions is effective to prevent an action potential in the neuron.

### Methods of Increasing Aversive Taste Tolerance

Disclosed herein include methods of increasing aversive taste tolerance in a subject in need thereof. In some embodiments, the method comprises: stimulating a plurality of prostaglandin receptor type 3 (Ptger3)-positive neurons in the lamina terminalis (LT^{Ptger3}-neurons) of the subject, thereby increasing aversive taste tolerance in the subject.

In some embodiments, the method comprises enhancing depolarization of the cell membranes of the plurality LT^{Ptger3}-neurons. Thus, stimulation of the plurality of LT^{Ptger3}-neurons can be enhanced, thus increasing ingestion of sodium, one or more aversive substances, or a combination thereof by the subject. In some embodiments, the method comprises at least one of enhancing cation influx into a cytosol of the plurality of LT^{Ptger3}-neurons, inhibiting anion influx into the cytosol of the plurality of LT^{Ptger3}-neurons, and inhibiting cation efflux from the cytosol of the plurality of LT^{Ptger3}-neurons. In some embodiments, the method comprises administering a vector encoding an ion modulator to the subject as described herein.

The sodium can be at a concentration of less than 120 mM (e.g., a low salt concentration). For example, the concentration of sodium can be 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM or a number or a range between any two of these values. The sodium can be at a concentration of greater than 300 mM (e.g., high salt concentration). For example, the concentration of sodium can be 300 mM, 400 mM, 500 mM, 600 mM, 700 mM, 800 mM, 900 mM, 1000 mM, or a number or a range between any two of these values, or greater.

Increasing aversive taste tolerance in the subject comprises increasing ingestion of sodium and one or more aversive substances by the subject, and wherein the sodium is at a concentration of less than 120 mM (e.g., 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM or a number or a range between any two of these values).

The concentration of any of the one or more aversive substances can vary. The one or more aversive substances can have a concentration of about 0.001 mM, 0.01 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 120 mM, 150 mM, 300 mM, 400 mM, 500 mM, 600 mM, 700 mM, 800 mM, 900 mM, 1000 mM, or a number or a range between any two of these values, or greater.

The one or more aversive substances can comprise one or more aversive minerals, one or more bitter-tasting substances, one or more sour-tasting substances, or any combination thereof. The one or more aversive minerals can comprise potassium chloride (KCl), calcium chloride (CaCl₂), and/or magnesium chloride (MgCl₂). The one or more bitter-tasting substances can comprise polyphenols, alkaloids, terpenoids, saponins, amino acids, bitter peptides, or any combination thereof. The one or more bitter-tasting substances can comprise quinine. The one or more sour-tasting substances can comprise lactic acid, citric acid, malic acid, acetic acid, or any combination thereof.

The method comprises, in some embodiments, determining sodium intake in the subject before stimulating the plurality of LT^{Ptger3}-neurons of the subject, determining sodium intake in the subject after stimulating the plurality of LT^{Ptger3}-neurons of the subject, or both. The extent to which the aversive taste tolerance (e.g., ingestion of the one or more aversive substances and/or sodium) is enhanced in the subject can vary. For example, the aversive taste tolerance of the subject can be enhanced by, by about, by at least, or by at least about, 10%, 20%, 30%, 40%, 50%, 60%, 70%, 80%, 90%, 95%, 98%, or a range between any two of these values, as compared to the subject before application of the method. The aversive taste tolerance of the subject can be increased by, by about, by at least, or by at least about 25% (e.g., 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) in the subject, e.g., as assessed by ingestion of the one or more aversive substances and/or sodium by the subject.

The LT^{Ptger3}-neurons can be in the subfornical organ of the LT (SFO^{Ptger3}-neurons), the vascular organ of lamina terminalis (OVLT^{Ptger3}-neurons), or both. The SFO^{Ptger3}-neurons can be GLUT1-expressing excitatory neurons. The SFO^{Ptger3}-neurons do not express GLUT2, GLUT3, GLUT4, and/or GLUT5.

Various methods/techniques can be used to stimulating LT^{Ptger3}-neurons. Stimulating the plurality of LT^{Ptger3}-neurons of the subject can comprise administration of a Ptger3-activator to the subject. The Ptger3-activator can increase Ptger3 expression, activity, or both. The Ptger3-activator can increase activity of a prostaglandin. The prostaglandin can be Prostaglandin E2 (PGE2). The Ptger3-activator can be a small molecule or a peptide. The peptide can comprise at least a portion of PGE2 or a functional fragment thereof, or a PGE2 mimetic. The Ptger3-activator can be administered to the subject orally or intravenously.

In some embodiments, the stimulating can comprise optogenetic stimulation, chemogenetic stimulation, or both. In some embodiments, stimulating the plurality of pre-LT^{Ptger3}-neurons of the subject comprises stimulating the plurality of LT^{Ptger3}-neurons by an conditional ion modulator. In some embodiments, stimulating the plurality of LT^{Ptger3}-neurons comprises: administering a nucleic acid encoding the conditional ion modulator to the subject, wherein the conditional ion modulator is activated in response to a stimulus or agonist; and applying the stimulus or agonist of the conditional ion modulator to the subject, causing the activation of the conditional ion modulator, thereby stimulating the plurality of LT^{Ptger3}-neurons.

The conditional ion modulator can comprise a channelrhodopsin-2 (ChR2), and the stimulus can comprise an optical stimulus. The conditional ion modulator can comprise a designer receptor exclusively activated by designer drug (DREADD), and the agonist can be clozapine-N-oxide (CNO). The increase in aversive taste tolerance can be detectable within 20 minutes of the onset of the chemogenetic stimulation. In some embodiments, stimulating the plurality of LT^{Ptger3}-neurons increases dopamine signaling in the central nervous system of the subject. In some embodiments, stimulating the plurality of LT^{Ptger3}-neurons does not increase appetitive drive for sodium in the subject.

The duration in which the plurality of LT^{Ptger3}-neurons is stimulated can vary. For example, a duration can be, 1 minute, 2 minutes, 3 minutes, 4 minutes, 5 minutes, 10 minutes, 20 minutes, 30 minutes, 40 minutes, 50 minutes, 100 minutes, 150 minutes, 200 minutes, or a range between any two of these values, or more. In some embodiments, the neuron is stimulated throughout the selected duration. In some embodiments, the neuron is stimulated with intervals of non-modulating break during the selected duration. In some embodiments, stimulating the plurality of LT^{Pger3}-neurons lasts for at least five minutes in total.

In some embodiments, the method comprises identifying the subject as in need of increasing aversive taste tolerance. For example, the subject can be identified as suffering from or at a risk of developing hyponatremia, excessive sweating, or a combination thereof. In some embodiments, the subject is a human or a non-human mammal. In some embodiments, the subject is an elderly subject. In some embodiments, the method of increasing aversive taste tolerance is a method of ameliorating, inhibiting, delaying the onset of, reducing the severity of, or preventing hyponatremia, excessive sweating, or a combination thereof.

### Identification of Modulators of Aversive Taste Tolerance

As used herein, a modulator of aversive taste tolerance refers to a compound (e.g., a small molecule compound, a nucleic acid, a protein, a lipid, a carbohydrate, or any combination thereof) that can partially or fully inhibit ingestion of sodium and/or one or more aversive substances; or a compound (e.g., a small molecule compound, a nucleic acid, a protein, a lipid, a carbohydrate, or any combination thereof) that can partially or fully enhance ingestion of sodium and/or one or more aversive substances. A modulator of aversive taste tolerance can be, for example, a modulator of LT^{Ptger}-neurons. A modulator of neurons (including, but not limited to, LT^{Ptger3}-neurons) can be a compound (e.g., a small molecule compound, a nucleic acid, a protein, a lipid, a carbohydrate, or any combination thereof) that can partially or fully inhibit the neurons; or a compound (e.g., a small molecule compound, a nucleic acid, a protein, a lipid, a carbohydrate, or any combination thereof) that can partially or fully stimulate the neurons.

The modulator of a neuron can be, for example, an activator (or stimulator) of the neuron which can partially or fully activate the neuron. For example, the activator can activate the neuron activity by, or by at least, 10%, 20%, 30%, 40%, 50%, 60%, 70%, 80%, 90%, 95%, 98%, 99%, or a range between any two of these values. In some embodiments, the activator can activate the neuron activity by, or by at least, 0.5-fold, 1-fold, 1.5-fold, 2-fold, 2.5-fold, 3-fold, or more, or a range between any two of these values.

The modulator of a neuron can also be, for example, an inhibitor of the neuron which can partially or fully inhibit the neuron. For example, the inhibitor can reduce the neuron activity by, or by at least, 10%, 20%, 30%, 40%, 50%, 60%, 70%, 80%, 90%, 95%, 98%, 99%, or a range between any two of these values. In some embodiments, the inhibitor can reduce the neuron activity by, or by at least, 0.5-fold, 1-fold, 1.5-fold, 2-fold, 2.5-fold, 3-fold, or more, or a range between any two of these values.

In some embodiments, identification of inhibitors of neurons of interest comprises screening compounds for their ability to act as antagonists to activate the neurons (e.g., LT^{Ptger3}-neurons). In some embodiments, compounds are also screened to determine whether or not they inhibit one or more known agonists for the neurons of interest, or whether or not they activate one or more known antagonists for the neurons of interest. Screening assays are well known in the art and can readily be adapted to identify antagonists of neurons, such as LT^{Ptger3}-neurons. Antagonists of a neuron, for example LT^{Ptger3}-neurons, may include compounds that interact with (e.g., bind to) an LT^{Ptger3}-neuron (e.g., a synaptic receptor on the neuron, e.g., Ptger3); compounds that bind to and block neurotransmitters from binding or by decreasing the amount of time neurotransmitters are in the synaptic cleft; compounds that block the synaptic connection of an LT^{Ptger3}-neuron with a neuron that is capable of being synaptically connected with the LT^{Ptger3}-neuron; or a combination thereof. The method disclosed herein, in some embodiments, comprises identifying the candidate compound as a suppressor for aversive taste tolerance if the candidate compound reduces valence toward sodium and/or one or more aversive substances of the subject compared to a control.

In some embodiments, identification of activators of neurons of interest (e.g., LT^{Ptger3}-neurons) comprises screening compounds for their ability to act as agonists to activate the neurons. In some embodiments, compounds are also screened to determine whether or not they activate one or more known agonists for the neurons, or whether or not they inhibit one or more antagonists for the neurons. Screening assays are well known in the art and can readily be adapted to identify agonists of neurons, such as LT^{Ptger3}-neurons. Agonists of a neuron, for example an LT^{Ptger3}-neuron, may include compounds that interact with (e.g., bind to) an LT^{Ptger3}-neuron (e.g., to a receptor localized to a synapse or dendrite of the neuron); compounds that block neuro transmitters from reentering the pre-synaptic axon terminal; compounds that enhance the synaptic connection of an LT^{Ptger3}-neuron with a neuron that is capable of being synaptically connected with the LT^{Ptger3}-neuron; or a combination thereof. In some embodiments, the modulator is an enhancer for aversive taste tolerance. The method disclosed herein, in some embodiments, comprises identifying the candidate compound as an enhancer for aversive taste tolerance if the candidate compound enhances valence toward sodium and/or one or more aversive substances of the subject compared to a control.

Disclosed herein include methods of identifying a modulator of aversive taste tolerance. In some embodiments, the method comprises: (a) contacting a candidate compound with a plurality of prostaglandin receptor type 3 (Ptger3)-positive neurons in the lamina terminalis (LT^{Ptger3}-neurons) to determine an electrophysiological response in the LT^{Ptger3}-neurons; (b) identifying the candidate compound as a modulator of the LT^{Ptger3}-neurons if the electrophysiological response in the LT^{Ptger3}-neurons contacted with the candidate compound is altered as compared to the electrophysiological response in the LT^{Ptger3}-neurons prior to contacting with the candidate compound; (c) administering the identified modulator of the LT^{Ptger3}-neurons to a subject; (d) assessing the change in valence toward sodium and/or one or more aversive substances of the subject in response to the administration of the identified modulator of the LT^{Ptger3}-neurons; and (e) identifying the identified modulator of the LT^{Ptger3}-neurons as a modulator for aversive taste tolerance if the identified modulator of the LT^{Ptger3}-neurons changes the valence toward sodium and/or one or more aversive substances of the subject compared to a control.

Many assays are known in the art to measure the electrophysiological response of a plurality of neurons. Examples of assays that can be used in the methods disclosed herein to measure, and thus determine alteration in an electrophysiological response in the LT^{Ptger3}-neurons, include, but are not limited to, a Ca²⁺ influx assay, a patch clamp assay, a calcium mobilization assay, a calcium imaging assay, an electrical signal detection assay, an assay based on fluorescent calcium sensor GCaMP6s, or a combination thereof.

Contacting the candidate compound with the plurality of LT^{Ptger3}-neurons can comprise administering the candidate compound to a subject comprising the LT^{Ptger3}-neurons via injection or via oral administration. The candidate compound can be, for example, a small molecule, peptide, a nucleic acid, or a combination thereof. The compounds that can be screened include, but are not limited to, small molecules (including both organic and inorganic molecules); peptides, proteins, antibodies and fragments thereof, and other organic compounds (e.g., peptidomimetics); nucleic acids; lipids; carbohydrates, or a combination thereof. For example, the candidate compounds can include, but are not limited to, soluble peptides, including members of random peptide libraries (see e.g., Lam, K. S. et al., 1991, Nature 354:82-84; Houghten, R. et al., 1991, Nature 354:84-86), and combinatorial chemistry-derived molecular libraries made of D- and/or L-configuration amino acids, phosphopeptides (including, but not limited to members of random or partially degenerate, directed phosphopeptide libraries; see e.g., Songyang, Z. et al., 1993, Cell 72:767-778), antibodies (including, but not limited to, polyclonal, monoclonal, humanized, anti-idiotypic, chimeric or single chain antibodies, and FAb, F(abN).sub.2 and FAb expression library fragments, and epitope-binding fragments thereof), and small organic or inorganic molecules, including libraries thereof. Other compounds that can be screened in accordance with the present application include, but are not limited to, small organic molecules, for example, those that are able to cross the blood-brain barrier. In some embodiments, the compounds that can be screened in accordance with the present application include, but are not limited to, small organic molecules, for example, those that are not required to cross the blood-brain barrier.

Many methods are available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). In some embodiments, chemical compounds to be used as candidate compounds can be synthesized from readily available starting materials using standard synthetic techniques and methodologies known to those of skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of known compounds, including natural products or synthetic chemicals, and biologically active materials, including proteins, can be screened for compounds which can activate or inhibit neurons of interest, for example LT^{Ptger3}-neurons. The molecular weight of the small molecule candidate compounds that can be screened for such activity can vary. For example, the small molecule can have a molecular weight of less than about 10 kD, about 8 kD, about 5 kD, about 2 kD, or a range between any two of these values. The small molecules can be, for example, naturally-occurring small molecules, synthetic organic or inorganic compounds, peptides and peptide mimetics. Small molecules in the present application are not limited to these forms. Extensive libraries of small molecules are commercially available, and a wide variety of assays are well known in the art to screen these molecules for the desired activity.

In some embodiments, modulators for aversive taste tolerance are identified from large libraries of natural product or synthetic (or semisynthetic) extracts or chemical libraries or from polypeptide or nucleic acid libraries, according to methods known in the art. Those of skill in the field of drug discovery and development will understand that the precise source of test extracts or compounds is not critical to the screening procedure(s) disclosed herein. Agents used in screens can include those known as therapeutics for the treatment of conditions such as anxiety, stress, itching, and/or pain. Virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al, Proc. Natl. Acad. Set U.S.A. 90:6909, 1993; Erb et al, Proc. Natl. Acad. Sci. USA 91: 11422, 1994; Zuckermann et al, J. Med. Chem. 37:2678, 1994; Cho et al, Science 261: 1303, 1993; Carrell et al, Angew. Chem. Int. Ed. Engl. 33:2059, 1994; Carell et al, Angew. Chem. Int. Ed. Engl. 33:2061, 1994; and Gallop et al, J. Med. Chem. 37: 1233, 1994. Furthermore, if desired, any library or compound is readily modified using standard chemical, physical, or biochemical methods.

### EXAMPLES

Some aspects of the embodiments discussed above are disclosed in further detail in the following examples, which are not in any way intended to limit the scope of the present disclosure.

### Example 1

### Neural Control of Sodium Consumption and Taste Valence

Using transcriptomics state-to-cell-type mapping and neural manipulations, it is described herein that positive and negative valences of salt are controlled by anatomically distinct neural circuits in the mammalian brain.

The hindbrain interoceptive circuit regulates sodium-specific appetitive drive while behavioral tolerance of aversive salts is encoded by a dedicated class of neurons in the forebrain lamina terminalis (LT) expressing prostaglandin E2 (PGE2) receptor, Ptger3. These LT neurons regulate salt tolerance by selectively modulating aversive taste sensitivity partly through a PGE2-Ptger3 axis. These results reveal the bimodal regulation of appetitive and tolerance signals toward salt, which together dictate the amount of sodium consumption under different internal states.

The same sensory stimuli often drive distinct behavioral responses depending on the internal nutrient state. Such state-dependent changes in nutrient value represent a fundamental basis of energy and fluid homeostasis. As the most abundant extracellular cation, sodium has critical roles in many physiological functions, including osmoregulation and neural transduction. While sufficient sodium ingestion is vital for animals, overconsumption can cause acute and chronic disorders, e.g., hypertension. To achieve optimal ingestion, the brain modulates sodium saliency (palatability) based on internal state and salt concentration. Under sodium-sated conditions, animals show modest attraction toward low concentrations of sodium (<100 mM) and strong aversion toward higher concentrations (>300 mM). This behavioral preference drastically switches when sodium is depleted in the body. Sodium-depleted animals vigorously ingest salt at higher concentrations that are normally aversive. Thus, sodium depletion elevates both appetitive drive and behavioral tolerance toward salt. This internal-state- and concentration-dependent valence regulation allows animals to minimize sodium overconsumption under sated states while maximizing the chance of sodium ingestion under depletion. Such flexible ingestion requires the brain to precisely control both appetitive drive and aversion tolerance toward salt.

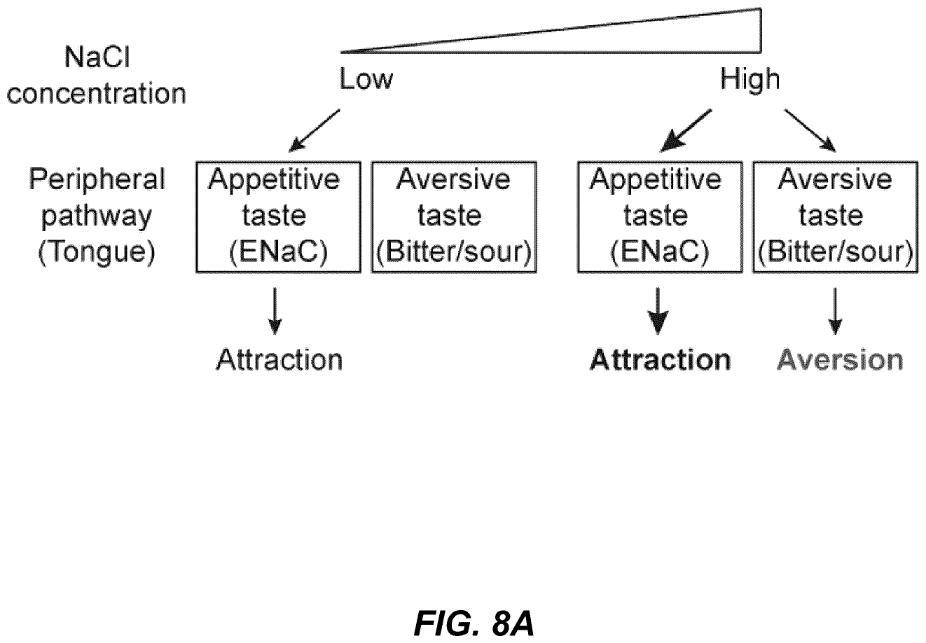

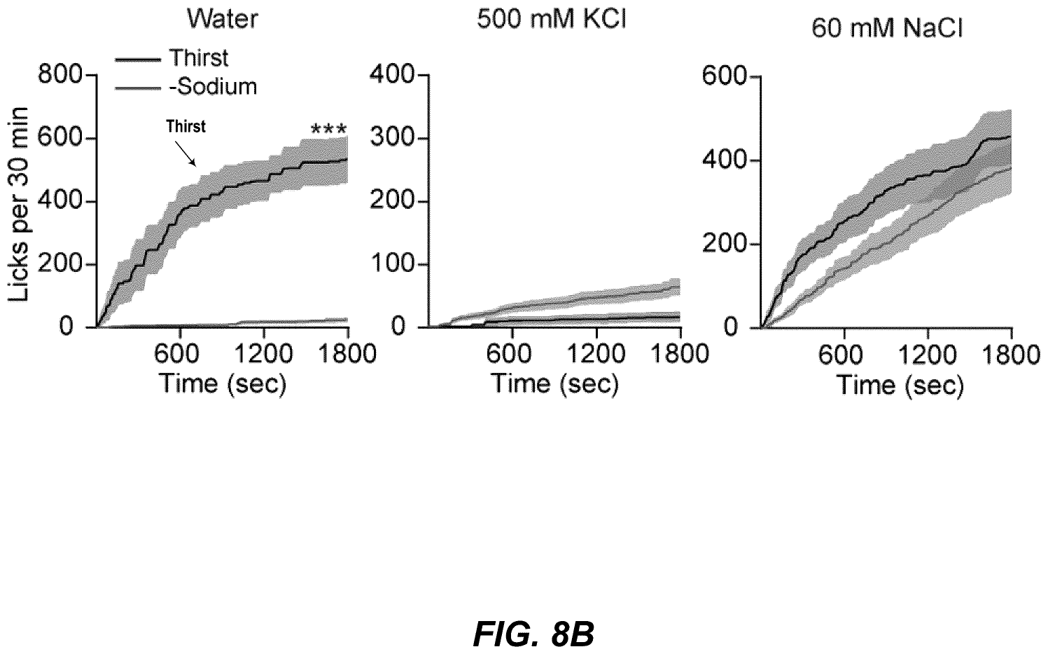

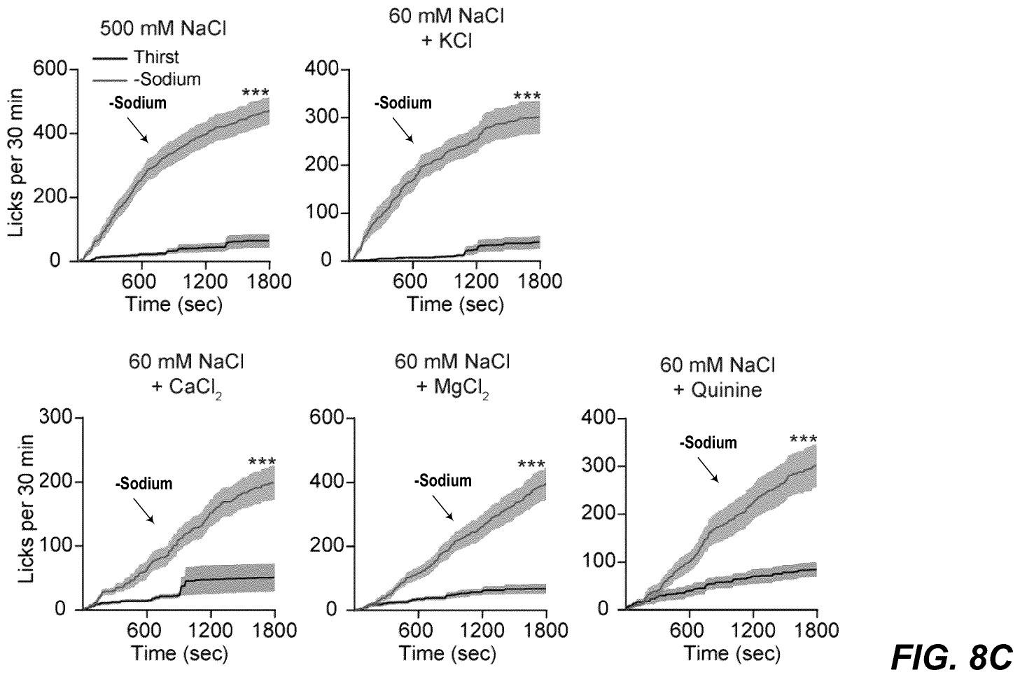

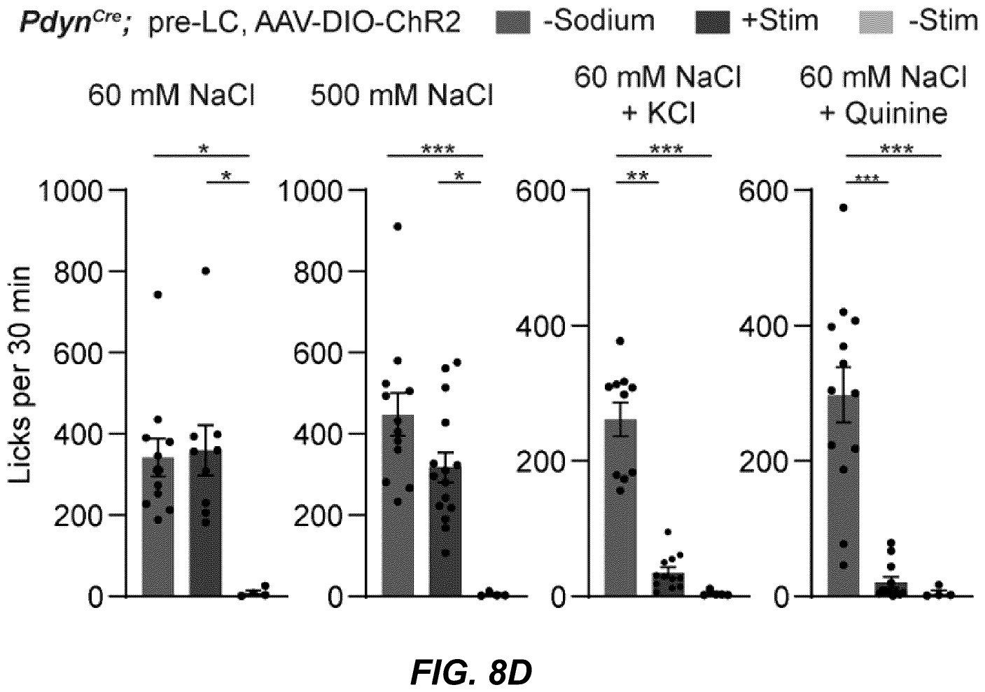

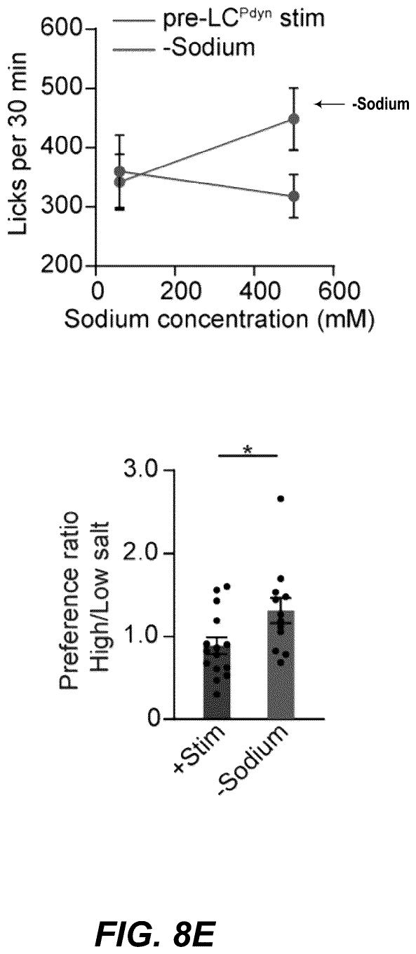

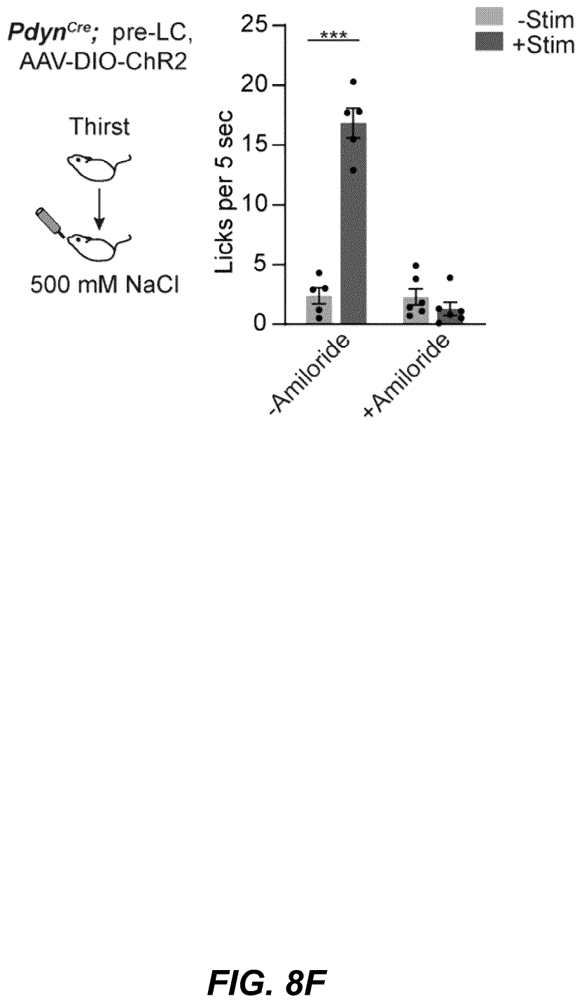

At the peripheral level, salts activate multiple taste pathways involved in appetitive and aversive behavioral responses. Low salt is detected by specific taste cells that express the epithelial sodium channel (ENaC). These signals mediate behavioral attraction under depleted conditions. Moderate to high concentrations of salt generally trigger behavioral aversion in sated animals by recruiting additional aversive taste pathways (FIG. 8A). Unlike the sodium-specific ENaC pathway, aversive salt pathways are promiscuous and activated by salts in general.

At the central level, sodium ingestion is controlled by at least two neural modules in the forebrain and hindbrain. First, specific neurons in the solitary nucleus tract (NTS) respond to internal sodium depletion through the action of aldosterone and angiotensin. These signals are transmitted to their downstream brain areas, including the pre-locus coeruleus (pre-LC), to drive salt-ingestive behavior. Second, sodium depletion activates neurons in the sensory organs of forebrain LT comprised of the subfornical organ (SFO) and organum vasculosum of the LT (OVLT). Although the genetic identity of the forebrain neurons is unknown, they were suggested to contribute to sodium preference. Despite the accumulating evidence on interoceptive circuits, it has been unclear how the brain modulates positive and negative sensory valences in an internal-state-dependent manner.

### Results

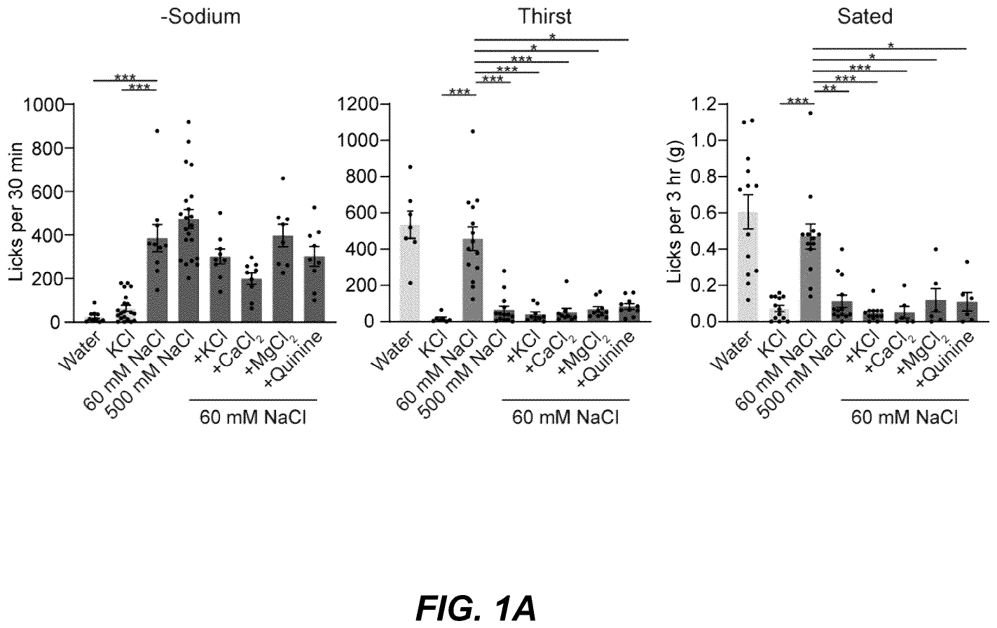

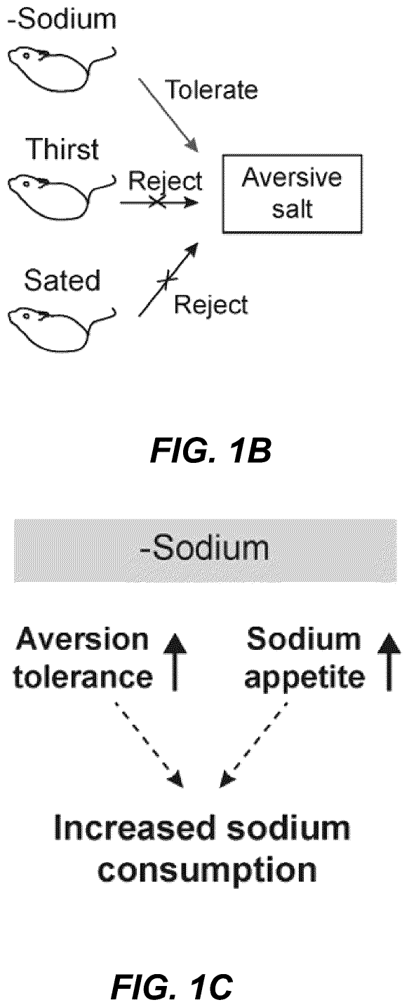

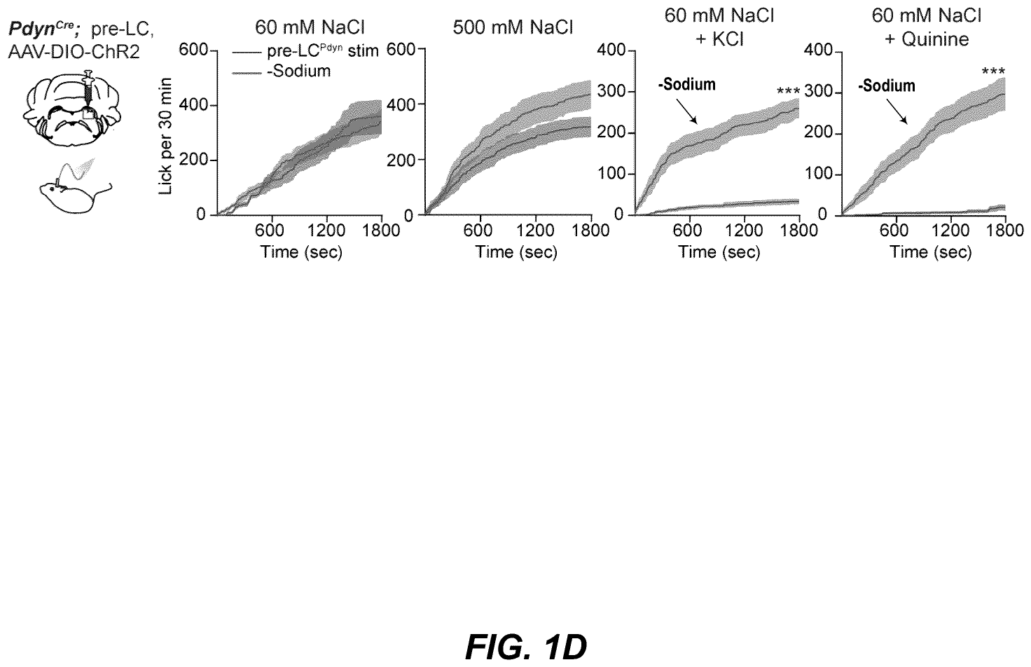

Toward circuit-level understanding of sodium homeostasis, it was examined whether appetitive drive and behavioral tolerance toward salt are independently regulated. Under sodium-depleted conditions, animals robustly consume both low (60 mM) and high (500 mM) salt solutions (FIG. 1A and FIG. 8B-FIG. 8C). Notably, these mice tolerate low salt containing aversive minerals such as KCl, CaCl₂ and MgCl₂ that frequently coexist with sodium in natural resources. In sharp contrast, sated or thirsty animals only accepted pure water and low salt with no tolerance toward aversive minerals (FIG. 1A and FIG. 8B-FIG. 8C). Similar results were obtained with a bitter compound: sodium-depleted animals consumed low salt supplemented with quinine, which was totally refused by sated or thirsty animals. These results show that sodium depletion regulates sensory valence of salt in two ways: 1) enhancing sodium-specific appetitive drive, and 2) suppressing behavioral aversion toward aversive compounds (FIG. 1B-FIG. 1C).

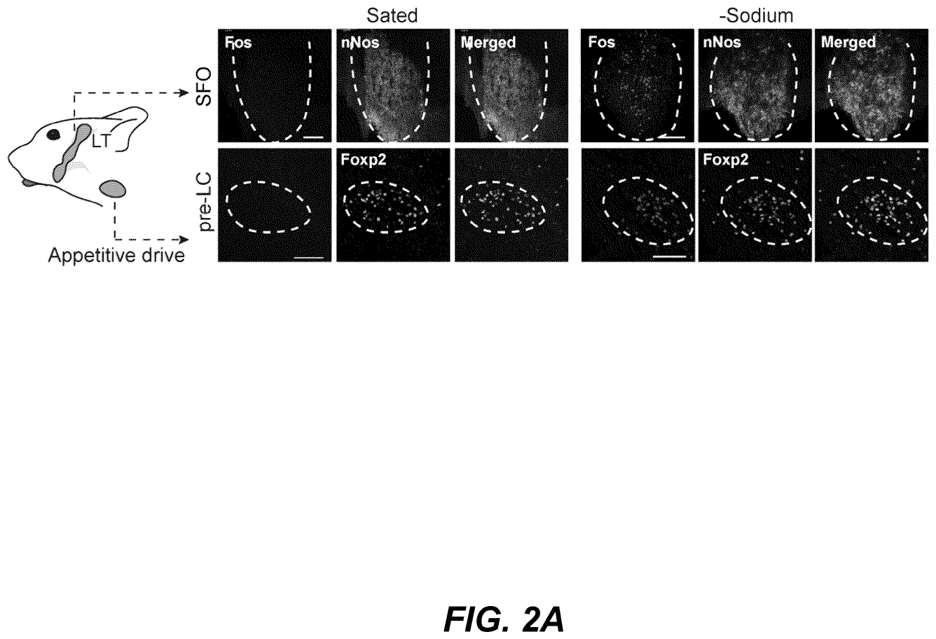

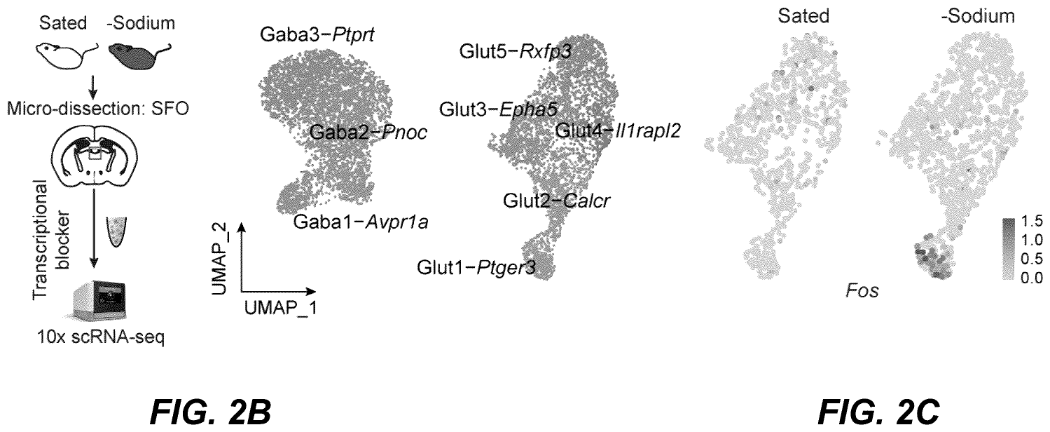

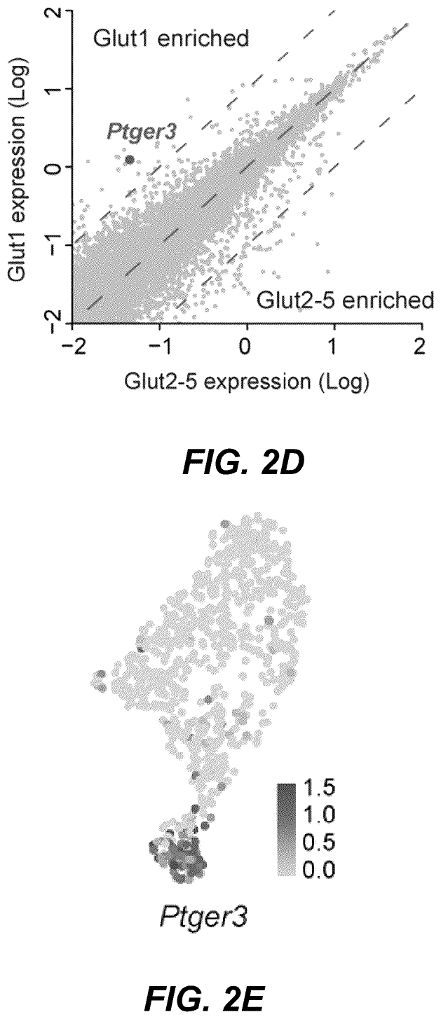

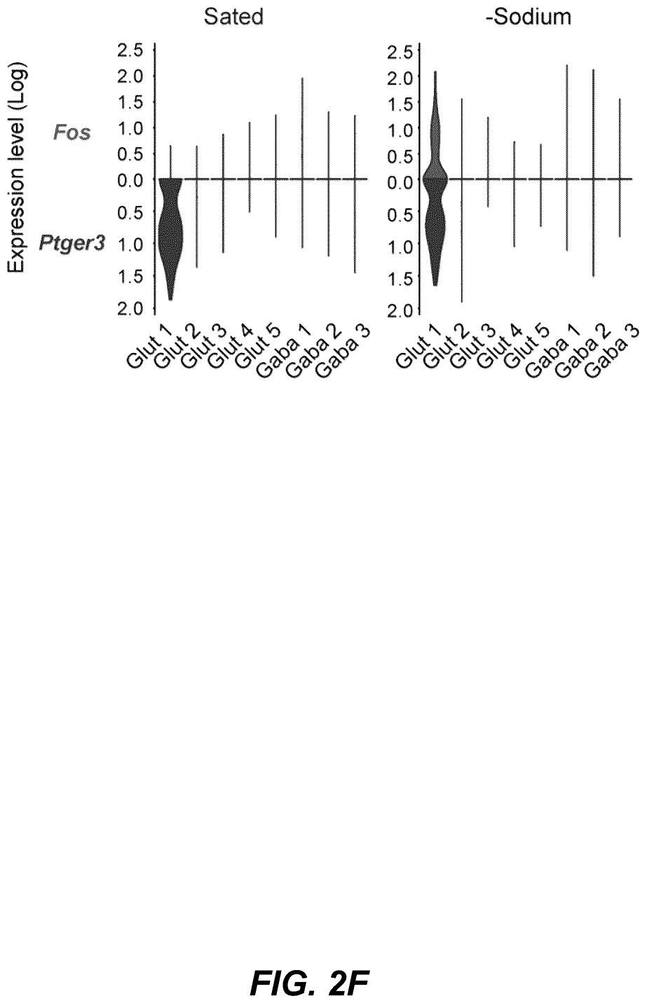

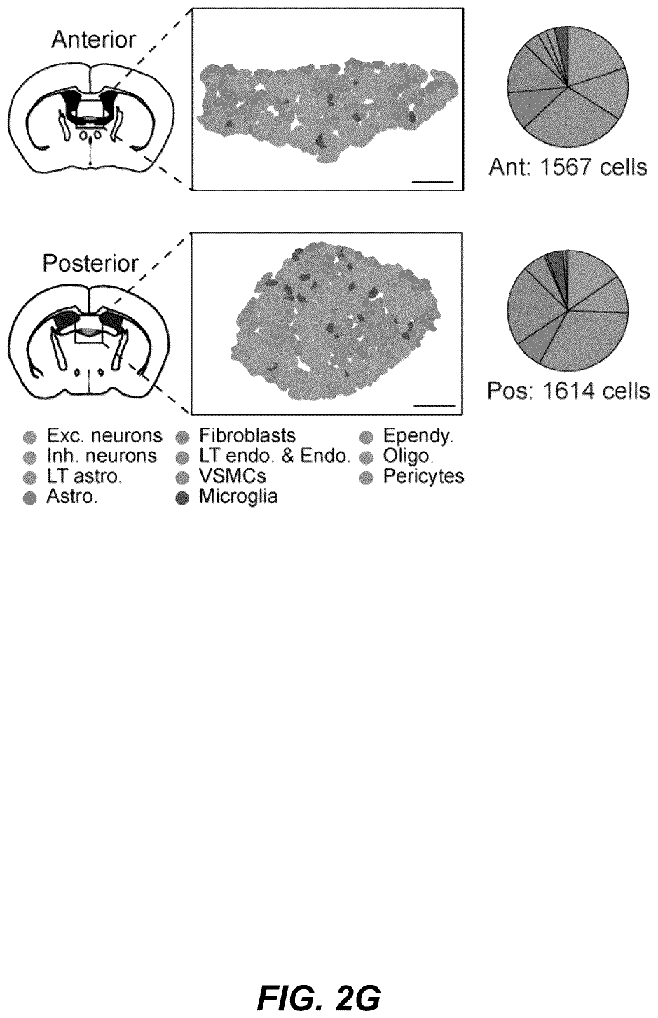

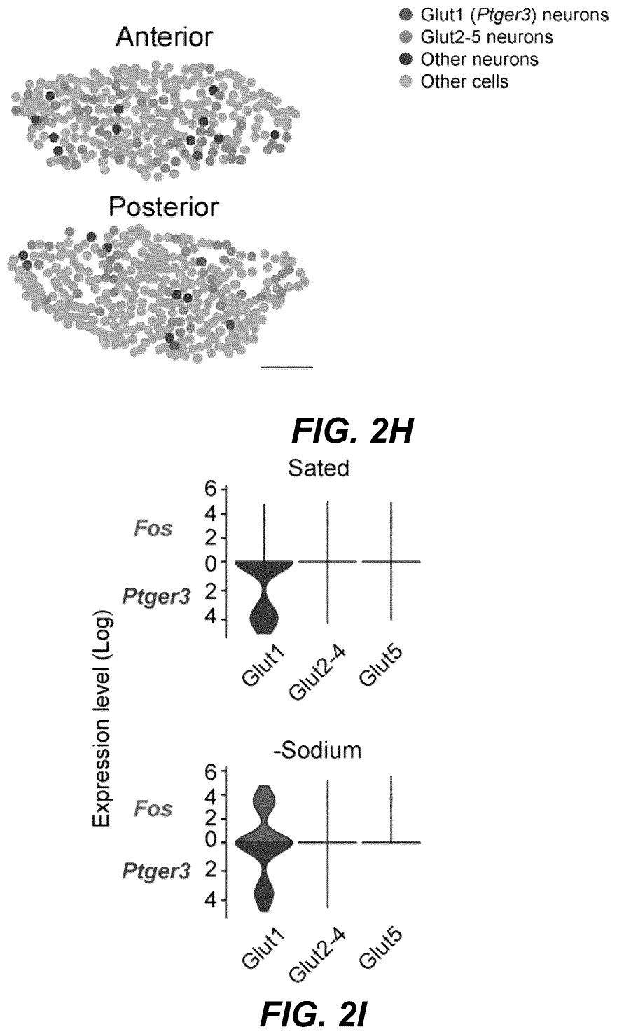

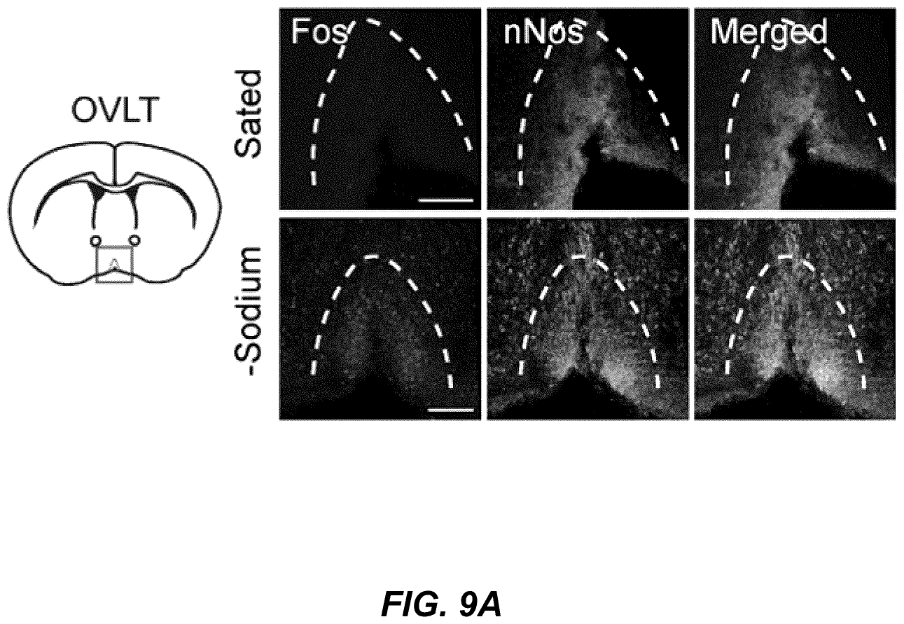

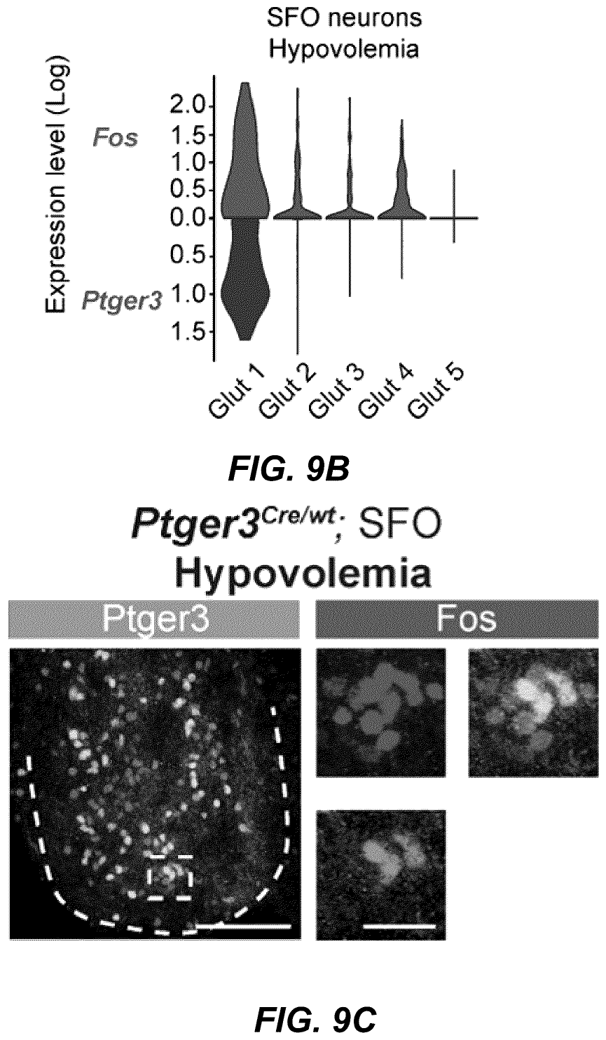

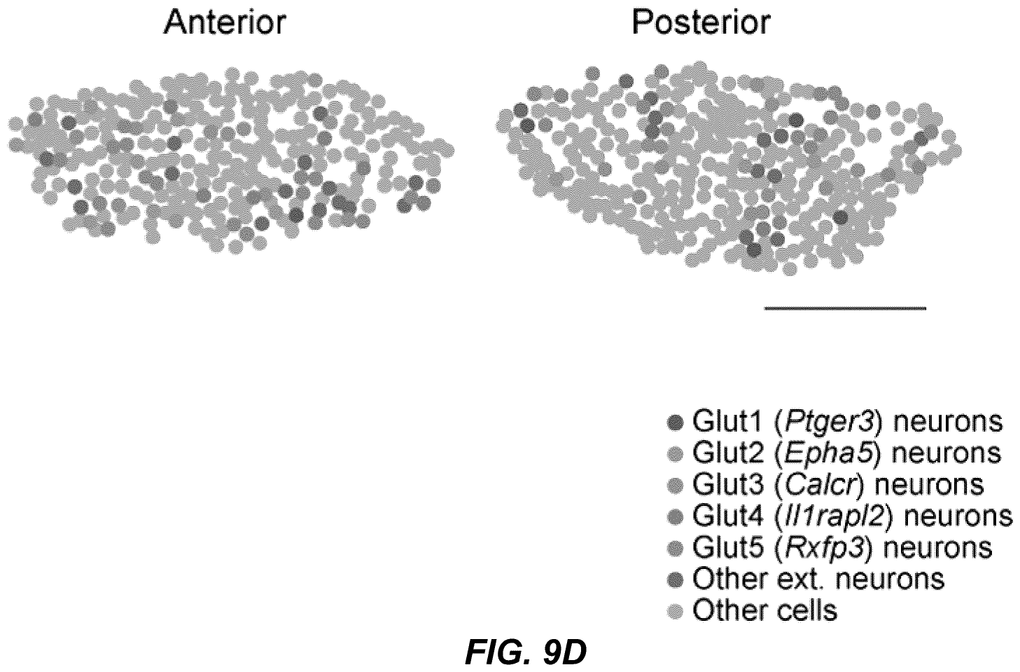

It was investigated if these two sensory modulations are controlled by independent neural circuits. Specific excitatory neurons in the hindbrain pre-LC (pre-LC^{Pdyn}) neurons receive inputs from NTS interoceptive neurons and are causally linked to sodium ingestion. It was investigated whether pre-LC^{Pdyn} neurons mediate both appetitive drive and aversion tolerance or only one of the two aspects. Channelrhodopsin (ChR2) was expressed in pre-LC^{Pdyn} neurons using adeno-associated virus (AAV) carrying Cre-dependent ChR2, and animals were tested with different concentrations of salt with or without aversive taste (FIG. 1D). As expected, both sodium-depleted and pre-LC^{Pdyn}-stimulated animals consumed low and high salt (FIG. 1D and FIG. 8D, 60 and 500 mM NaCl). However, compared to sodium depletion, pre-LC^{Pdyn}-stimulated animals preferred low salt over high salt despite stronger appetitive signals through the ENaC pathway by high salt (FIG. 8E). These results suggest that pre-LC^{PdY}-mediated sodium intake may lack the normal salt tolerance observed under sodium depletion. Indeed, only sodium-depleted, but not pre-LC^{Pdyn}-stimulated animals tolerated aversive KCl or quinine in low salt solutions (FIG. 1D and FIG. 8D, 60 mM NaCl with KCl or quinine). Furthermore, in the presence of a potent ENaC blocker, amiloride, photostimulation of pre-LC^{Pdyn} neurons no longer induced sodium consumption (FIG. 8F). These behavioral results demonstrate that the hindbrain circuit mainly controls sodium-specific appetitive drive, and that the attraction and aversion components toward salt are separable in the brain.

Where is aversive salt tolerance encoded in the brain?Besides the hindbrain interoceptive circuit, the forebrain LT has been suggested to regulate sodium intake. Given sodium-specific appetite regulation by the hindbrain circuit, it was hypothesized that forebrain LT neurons regulate aversive taste modulation and behavioral tolerance toward salt. In accordance with previous work, sparse but robust activation of LT neurons under sodium depletion was observed based on immediate early gene expression (FIG. 2A and FIG. 9A). To identify specific cell types activated under sodium depletion, stimulus-to-cell-type mapping on the 10× Chromium platform was adapted. Transcriptomic data between sated and sodium-depleted conditions was compared by collecting the SFO, enzymatically dissociating the tissue, and performing scRNA-seq experiments in the presence of a transcription blocker. Approximately 40,000 cells were collected from the SFO of sated and sodium-depleted animals, of which ˜10,000 neurons were used for further analyses. The data from sodium-depleted animals were compared with those from sated animals (FIG. 2B). Among all neurons, sodium depletion activated a single excitatory neuron type (Glut1) (FIG. 2C). Gene expression comparison between neuron types (FIG. 2D) identified that prostaglandin receptor type 3 mRNA (Ptger3) was selectively expressed in the sodium-depletion-activated Glut1 cluster (FIG. 2E-FIG. 2F). Interestingly, Ptger3-positive neurons (SFO^{Pt}*3) comprise a small population of neuron types activated under hypovolemic thirst, a condition where both water and salt are depleted (FIG. 9B-FIG. 9C).

To test if the SFO^{Ptger3} population is anatomically distinct in the SFO, spatial transcriptomic analyses using seq-FISH was performed. 192 genes were selected for mRNA hybridization based on transcriptomic data and annotated them in the anterior and posterior areas of the SFO (FIG. 2G). By analyzing ˜1700 neurons, it was validated that Ptger3-expressing neurons are anatomically distinct from other neural populations (FIG. 2H and FIG. 9D). Moreover, SFO^{Ptger3} neurons were the only active population under sodium depletion (FIG. 2I), in accordance with the scRNA-seq results.

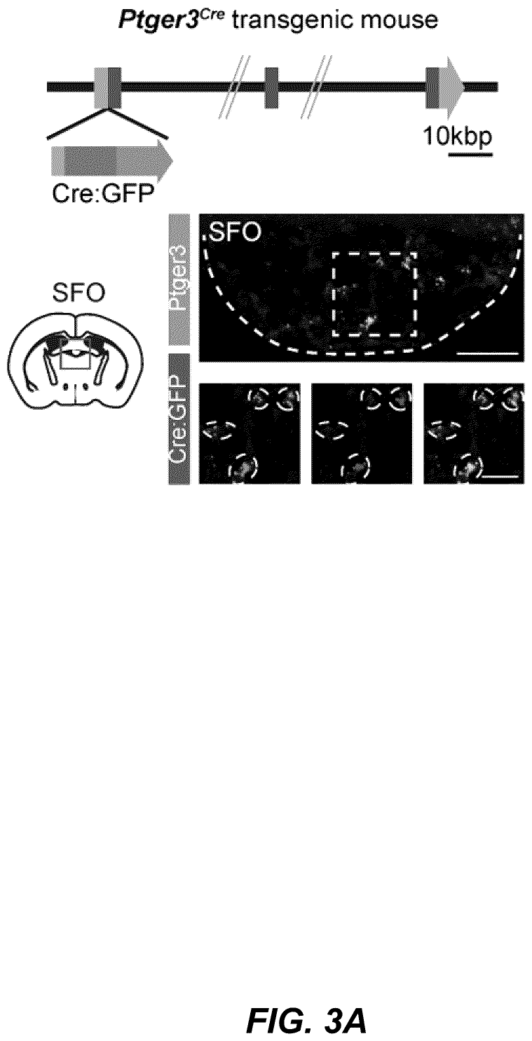

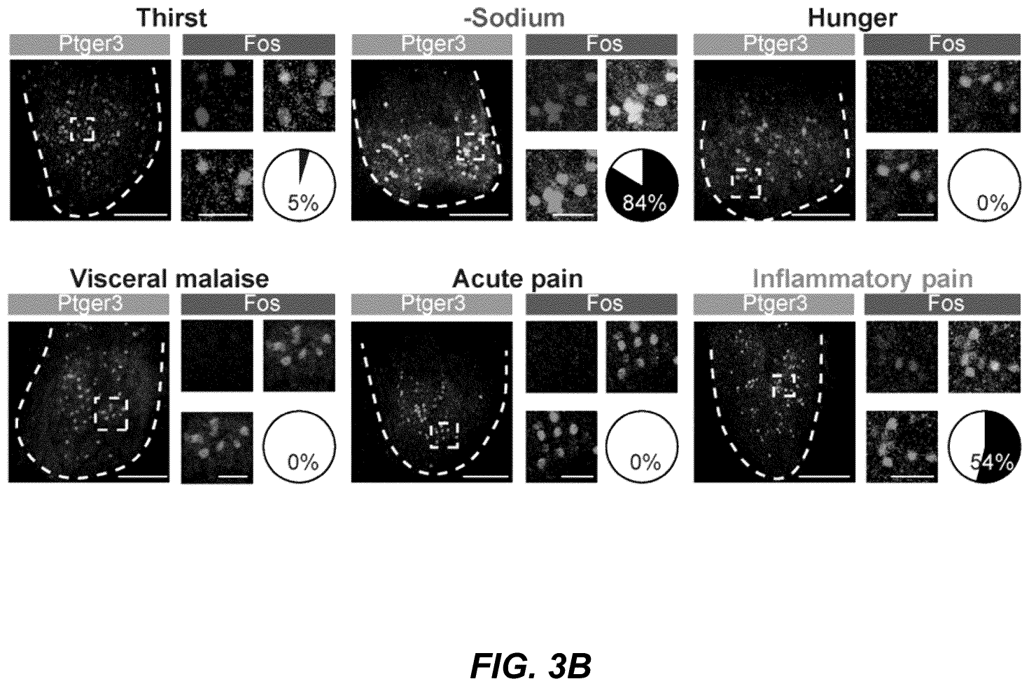

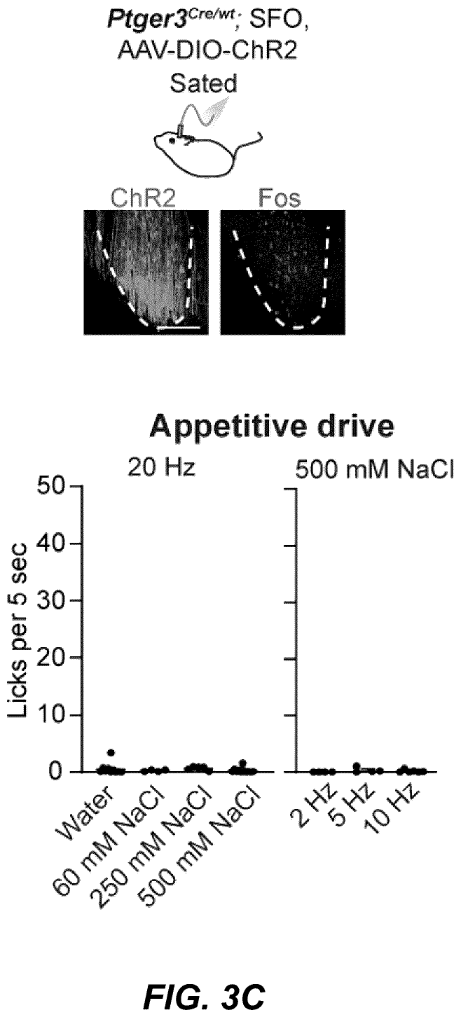

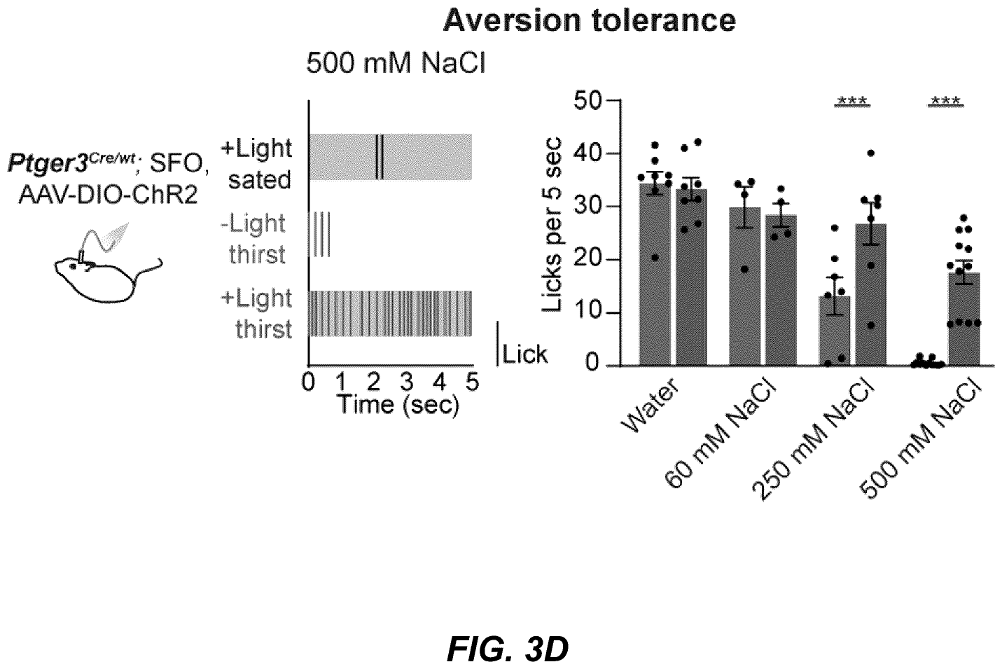

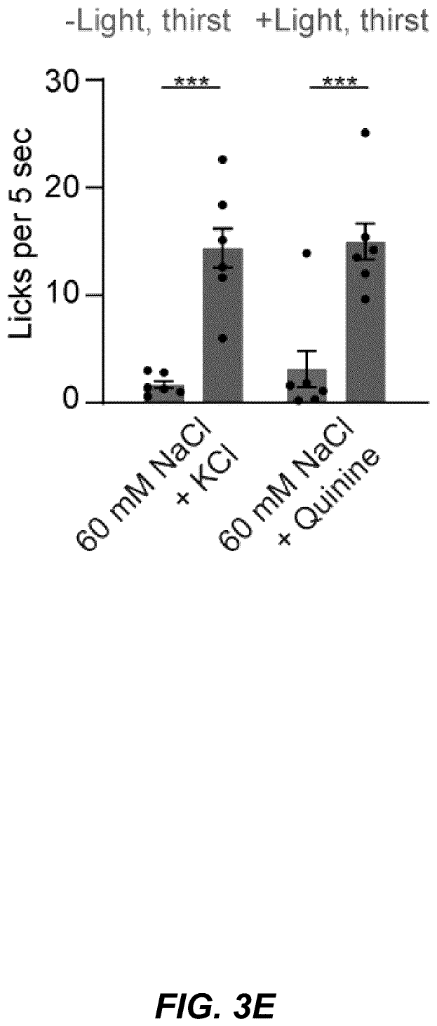

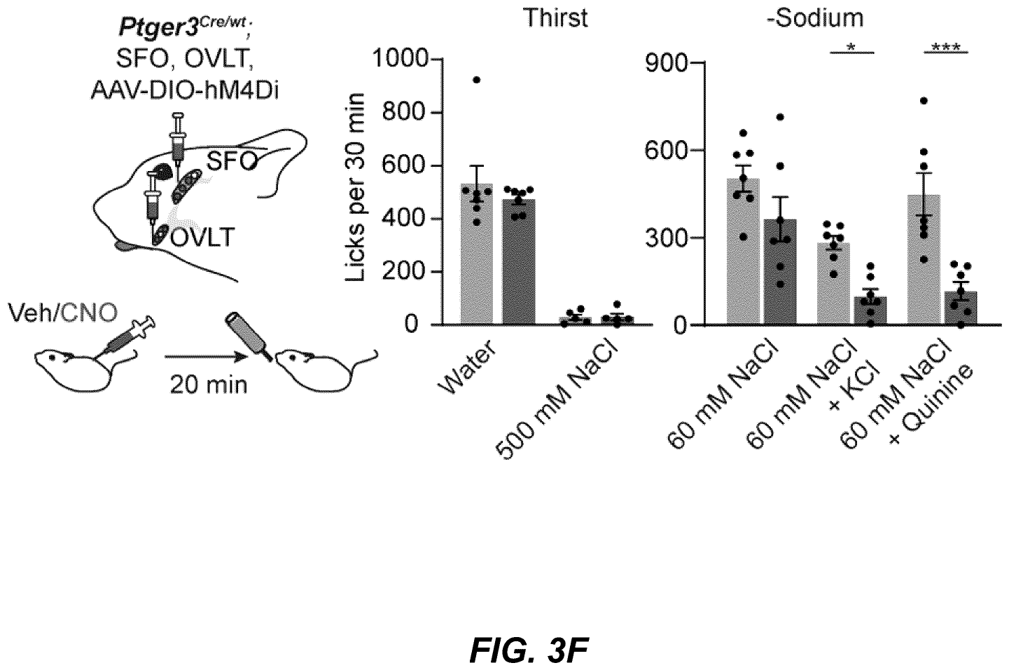

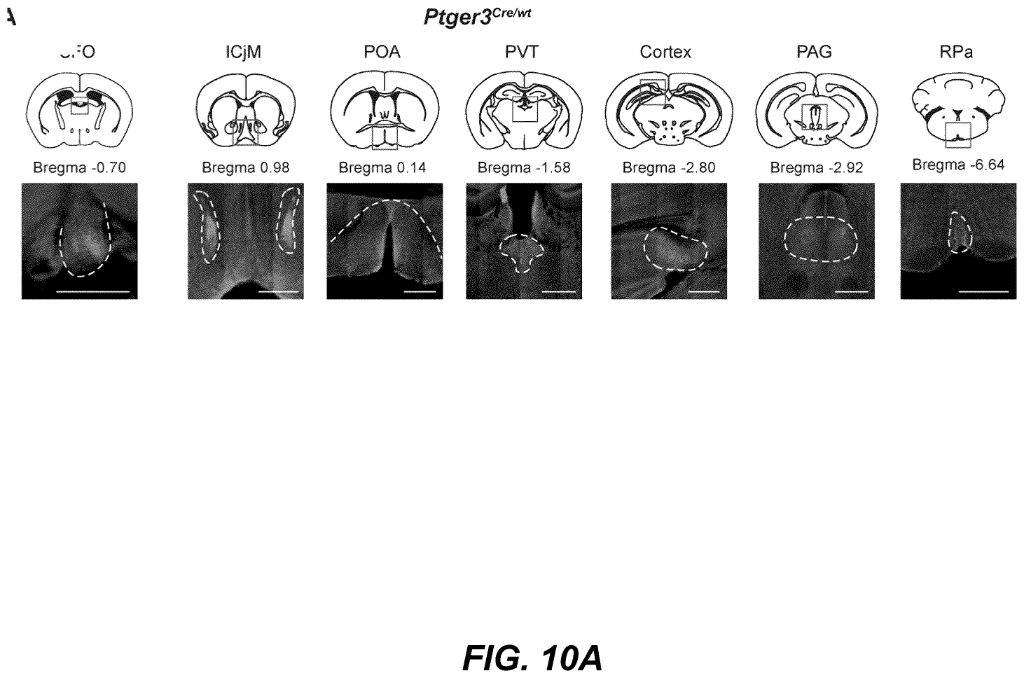

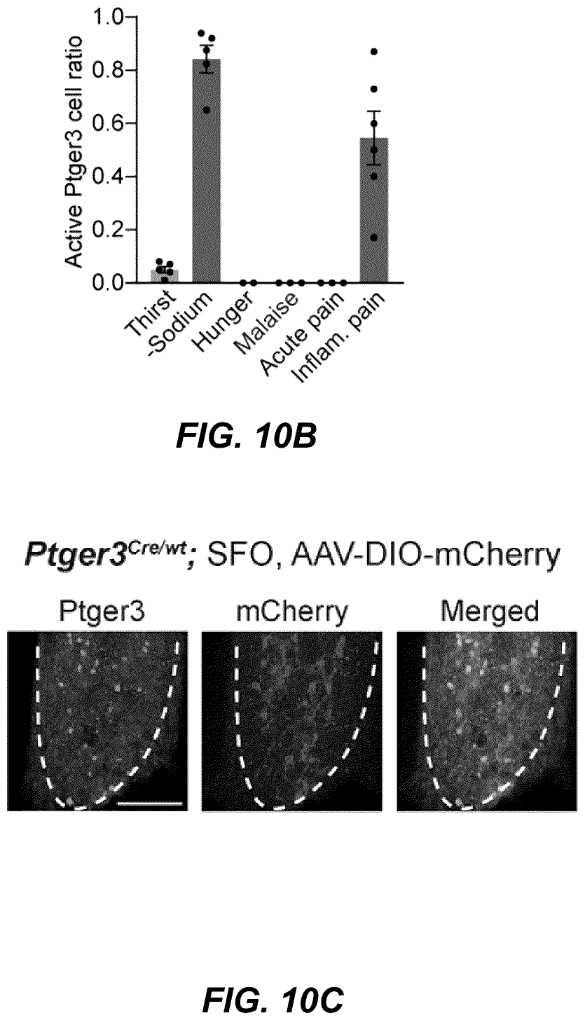

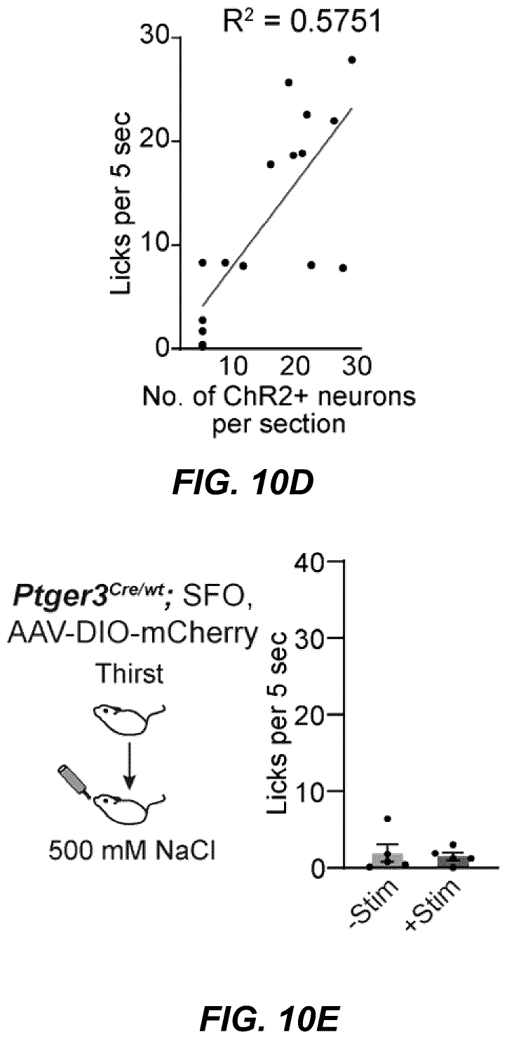

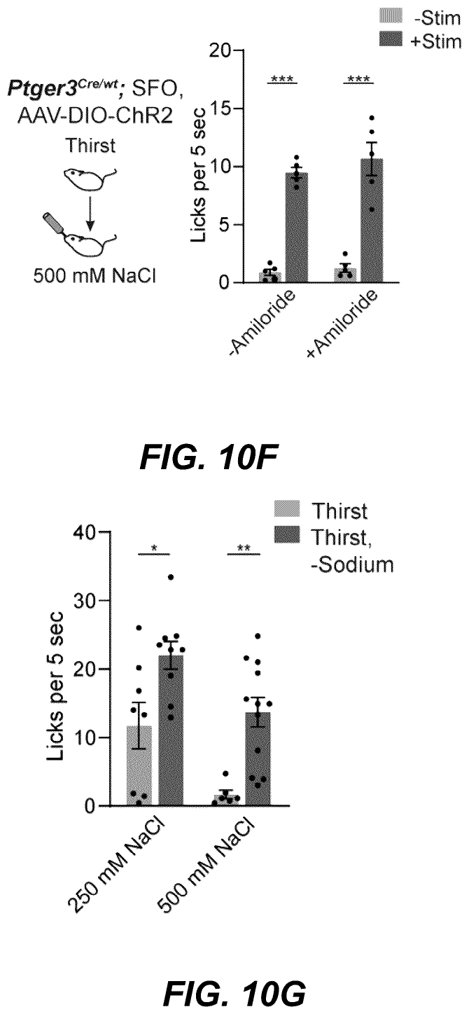

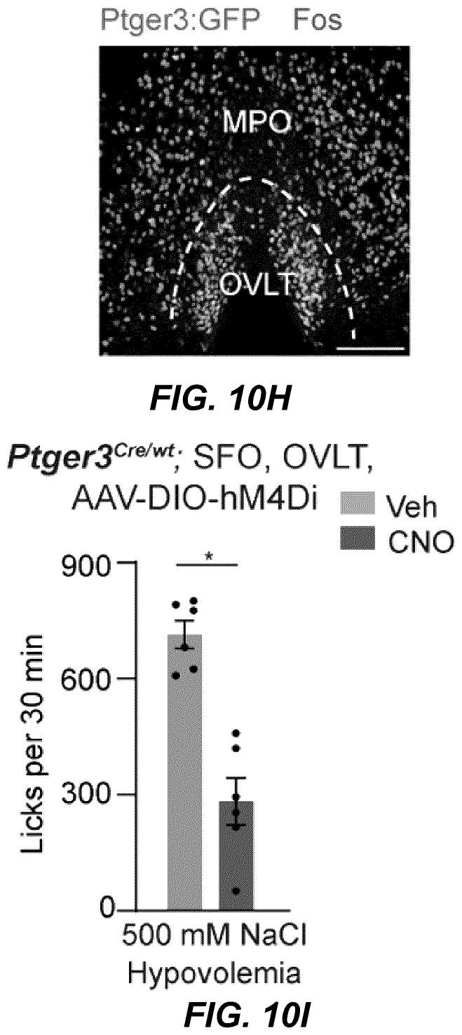

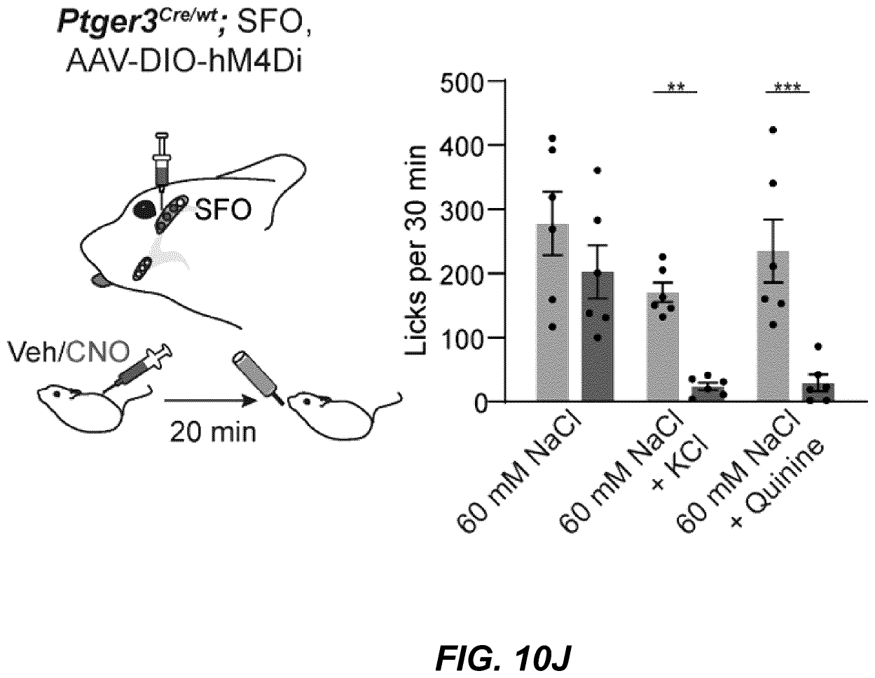

To gain genetic access to Ptger3-positive neurons, transgenic mice were generated with IRES-Cre recombinase:GFP fusion protein targeted to the Ptger3 locus (abbreviated as Ptger3^{Cre}, also see FIG. 10A). Fluorescent in situ hybridization validated faithful Cre expression in endogenous Ptger3-expressing neurons (FIG. 3A). To test the activation spectrum of these neurons, animals were exposed to different internal states and stimuli including various nutrient depletion, visceral malaise, pain, and inflammation (FIG. 3B). Quantification of Fos immunofluorescence signals showed that SFO^{Ptger3} neurons are narrowly tuned to sodium-need states (FIG. 10B). Interestingly, the only exception was inflammatory pain, which partially activated these neurons.

In Ptger3^{Cre} transgenic line, gene-targeted Cre insertion results in premature Ptger3 disruption. Thus, heterozygous Ptger3^{Cre/wt} animals were used to test whether SFO^{Ptger3} neurons mediate appetitive drive and/or aversion tolerance toward salt. To this end, we infected AAV-DIO-ChR2 in the SFO of Ptger3^{Cre/wt} mice and implanted an optic fiber over the SFO (FIG. 10C). Although photostimulation strongly activated SFO^{Ptger3} neurons, it did not induce ingestive behavior toward fluids with any stimulation parameters tested (FIG. 3C). It was next examined whether these neurons mediate behavioral tolerance. While thirsty mice showed dose-dependent aversion toward salt, SFO^{Ptger3}-stimulated animals exhibited greatly increased consumption of aversive high salt (FIG. 3D, FIG. 10D-FIG. 10E). Importantly, photostimulation did not affect water or low-salt consumption, excluding the possibility that SFO^{Ptger3} neurons mediate general appetite modulation. The same photostimulation paradigm also enhanced behavioral tolerance toward aversive compounds presented with low salt (FIG. 3E). Unlike pre-LC^{Pdyn} neurons, amiloride had no effect on SFO^{Ptger3}-mediated salt tolerance (FIG. 10F). The levels of SFO^{Ptger3}-induced tolerance were comparable to those under sodium depletion FIG. 10G).

It was also investigated if LT^{Ptger3} neurons are functionally required for salt tolerance. Because a subset of OVLT neurons also expressed Ptger3 (FIG. 10H), and were activated under sodium depletion, we expressed an inhibitory DREADD in Ptger3 neurons of these areas by transducing AAV-DIO-hM4Di. Inhibition of SFO/OVLT^{Ptger3} neurons by a synthetic DREADD ligand, CNO, drastically suppressed tolerance toward aversive salt under sodium-depleted or hypovolemic state (FIG. 3F and FIG. 10I). LT^{Ptger3}-inhibited mice no longer tolerated low salt with quinine or KCl, whereas the same inhibition had no effect on solute intake under osmotic thirst conditions where Ptger3 neurons are not activated (FIG. 3F). The blunted tolerance was also observed when only SFO^{Ptger3} neurons were inhibited (FIG. 10J). Taken together, the neural perturbation experiments revealed an essential role of SFO^{Ptger3} neurons in regulating aversion tolerance, but not in appetitive drive toward salt.

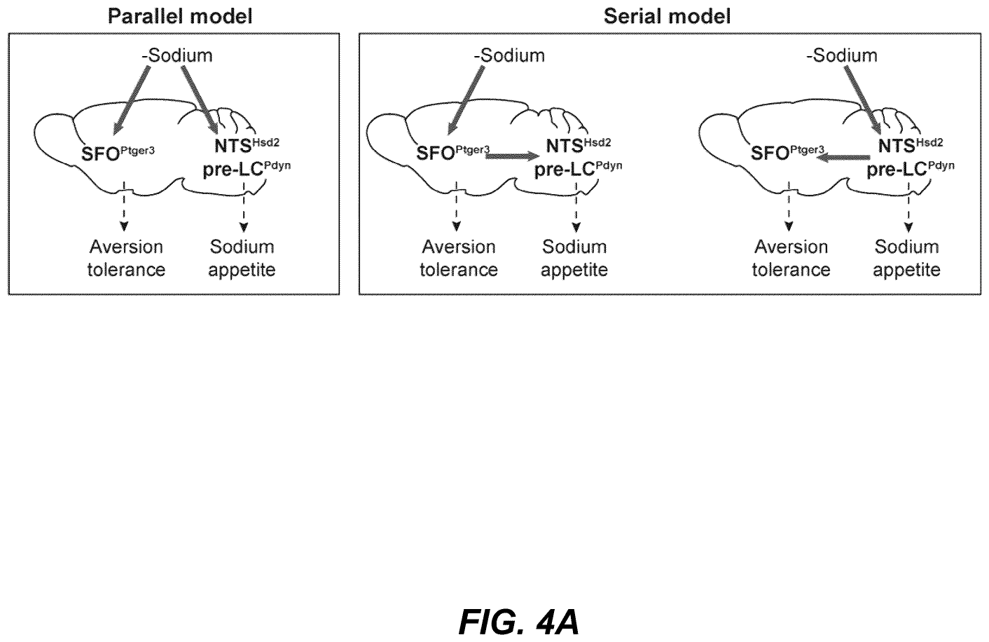

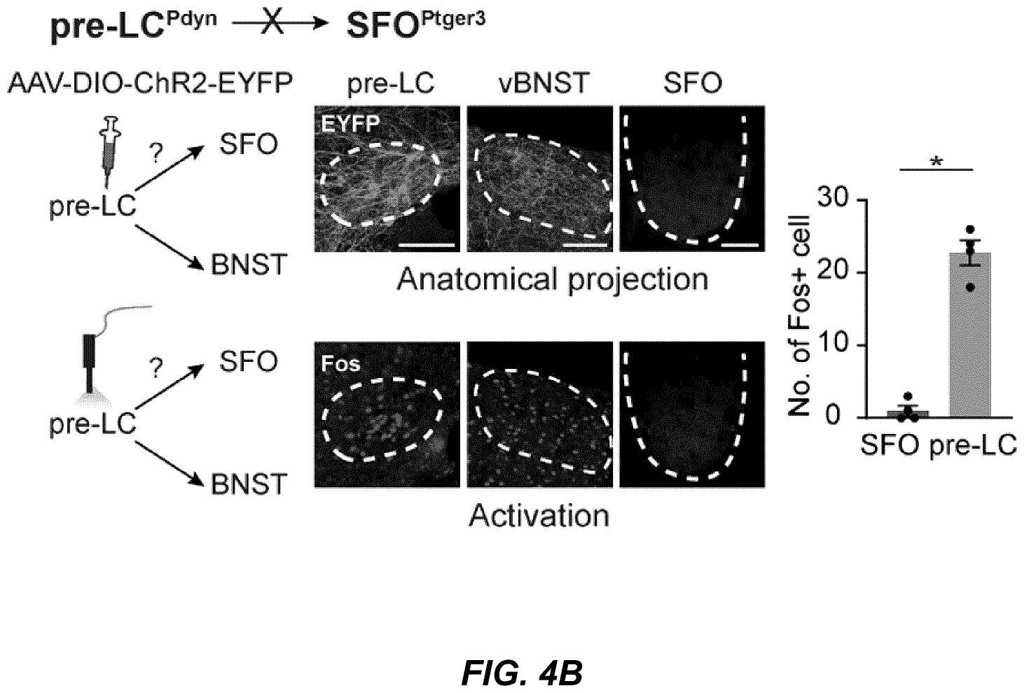

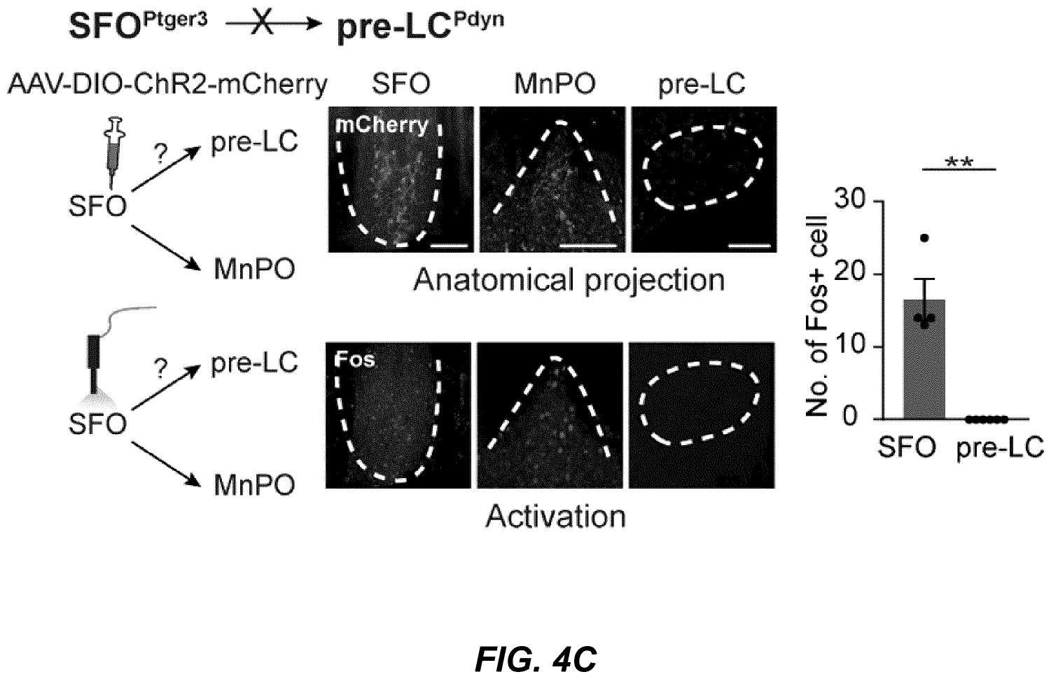

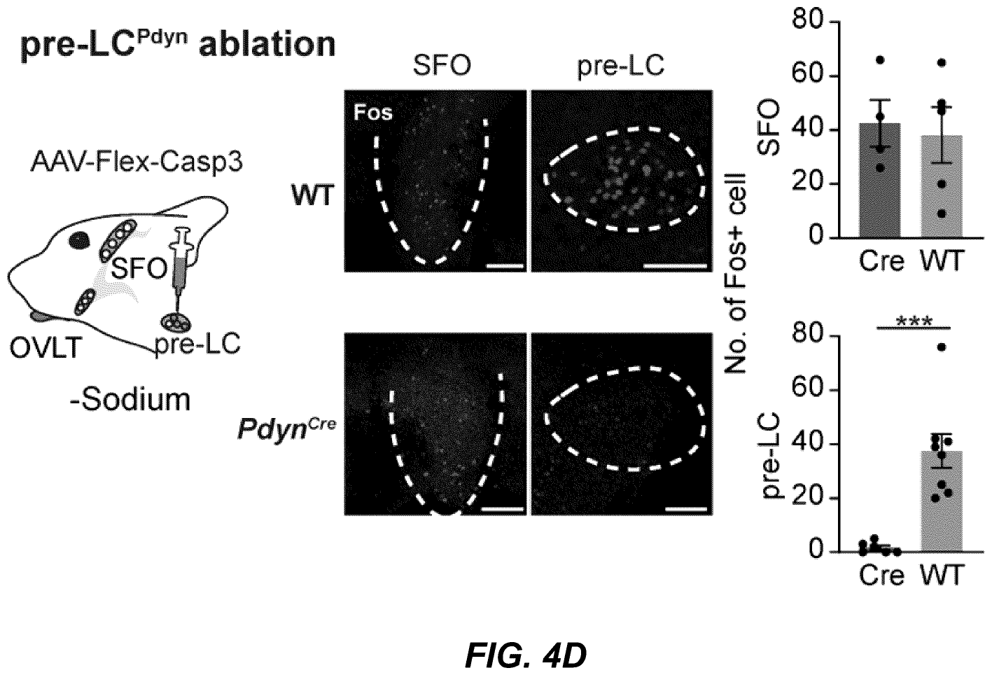

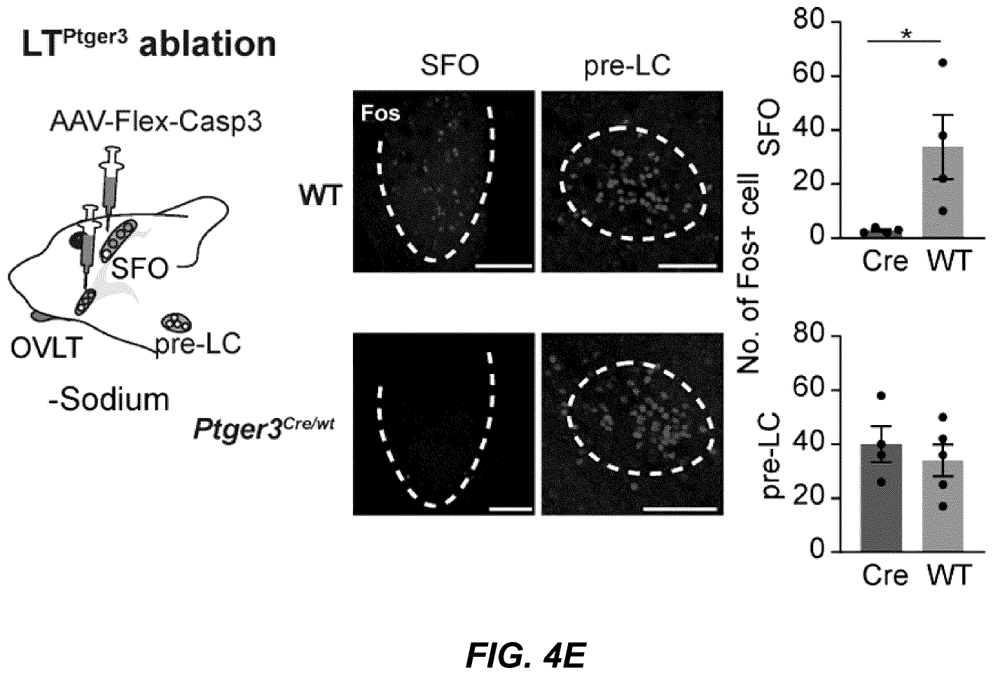

Given the distinct functions of forebrain and hindbrain circuits for salt ingestion, it was asked whether sodium depletion activates pre-LC^{Pdyn} and SFO^{Ptger3} neurons independently or in series (FIG. 4A). To test this, pre-LC^{Pdyn}→SFO^{tger3} and SFO^{Ptger3}→pre-LC^{Pdyn} projections were genetically probed by expressing ChR2-EYFP/ChR2-mCherry in either neural population. While both populations send axonal projections to downstream areas, practically no anatomical interaction was observed between pre-LC^{Pdyn} and SFO^{Ptger3} neurons (FIG. 4B-FIG. 4C, top). Optogenetic stimulation of each excitatory population did not activate neurons in the other nucleus, showing functional independence between these two neural populations (FIG. 4B-FIG. 4C, bottom). Moreover, ablation of either pre-LC^{Pdyn} or LT^{Ptger3} by Cre-dependent expression of caspase did not affect Fos immunofluorescence signals in the remaining nucleus under sodium depletion (FIG. 4D-FIG. 4E). Thus, forebrain and hindbrain circuits are independently activated under depleted states.

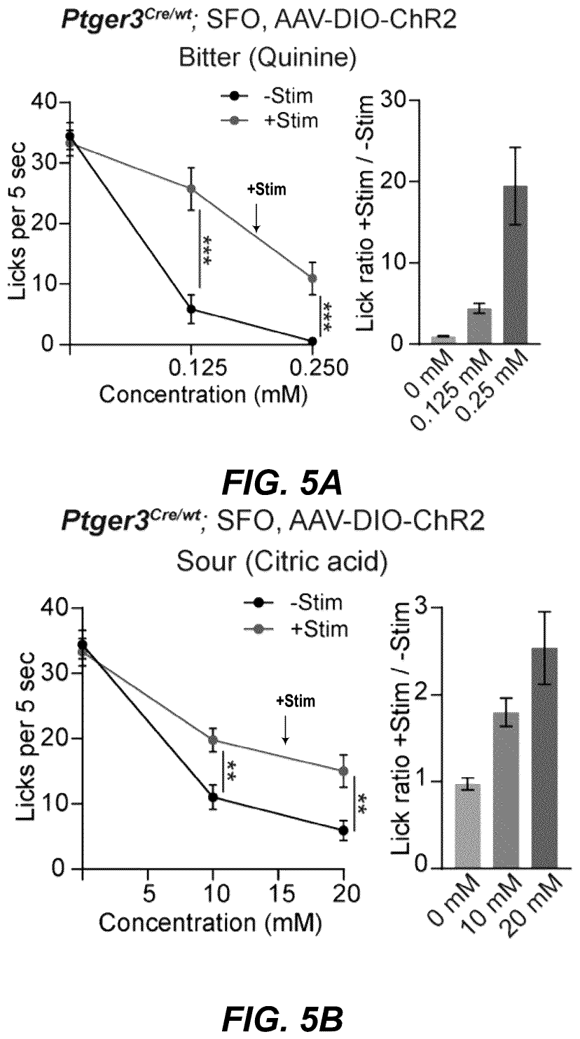

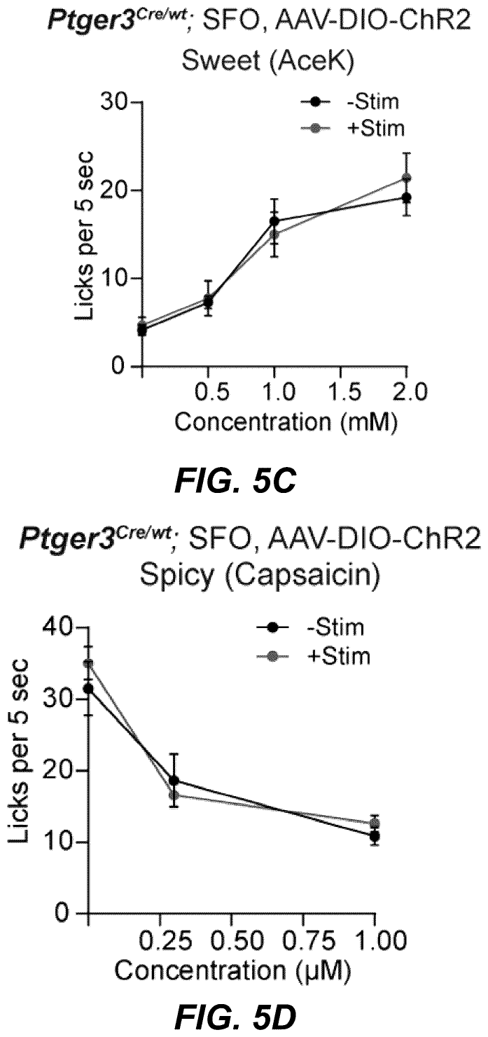

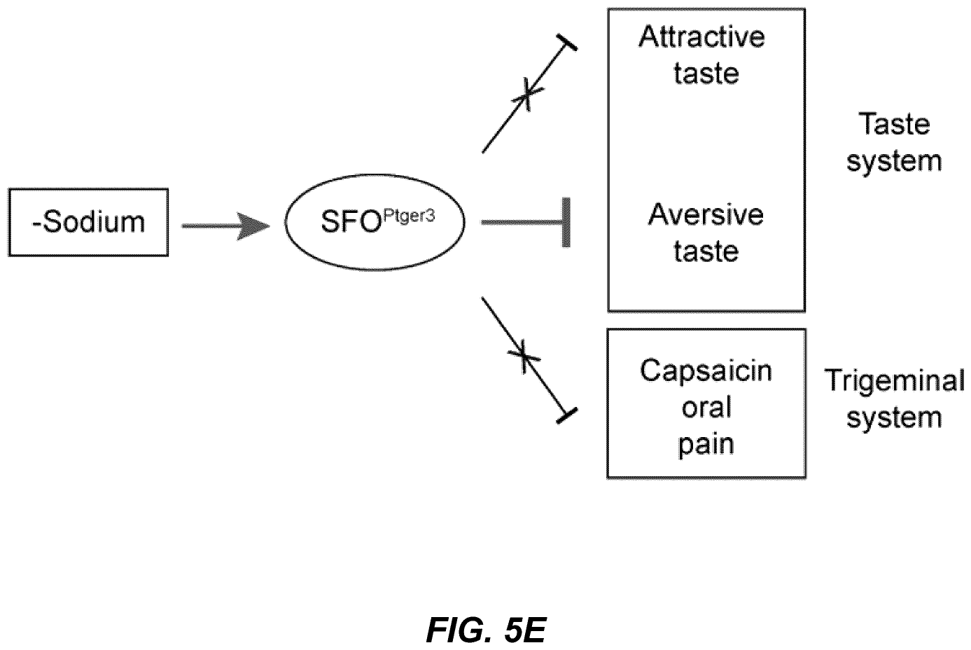

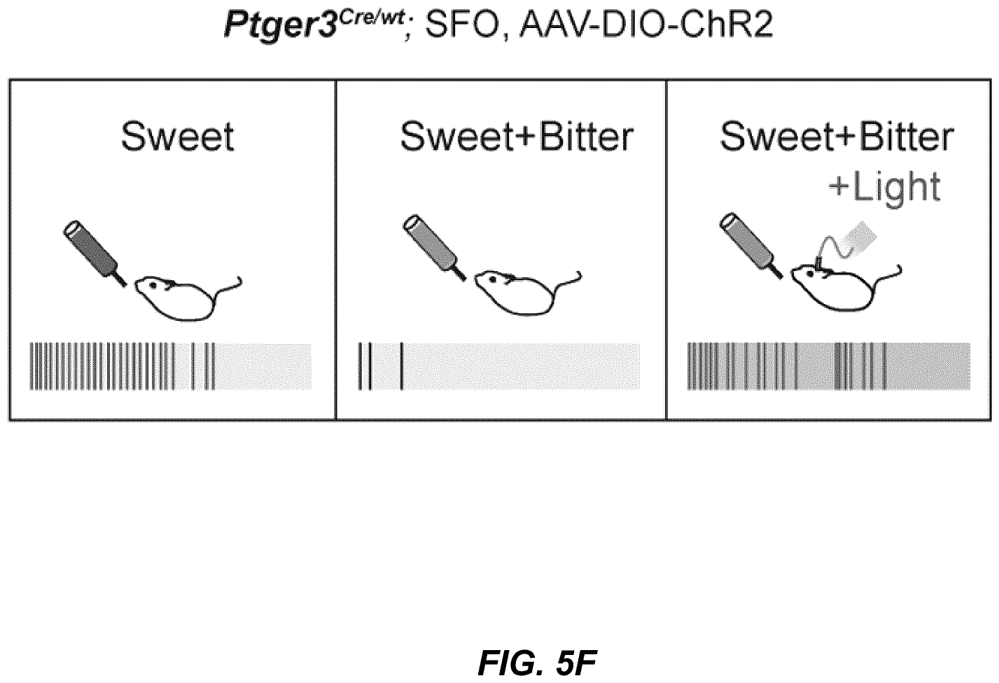

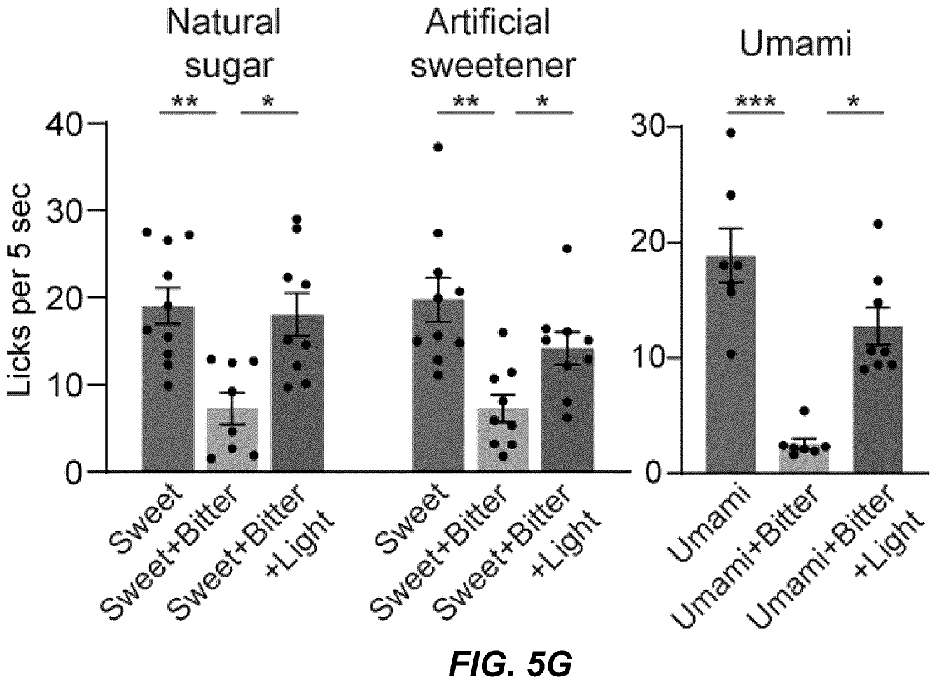

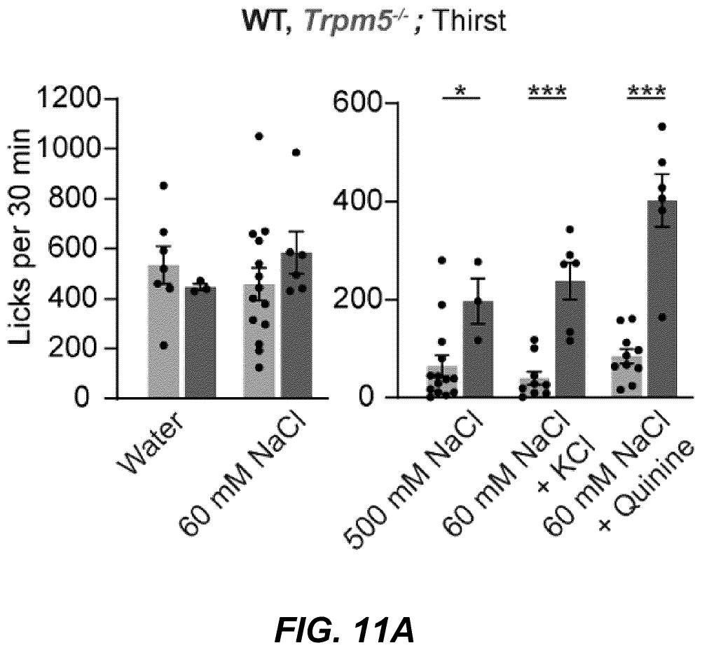

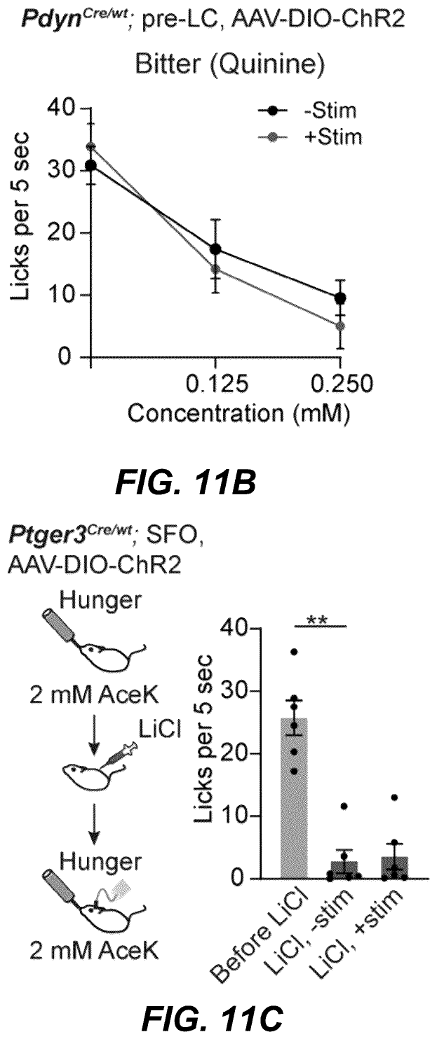

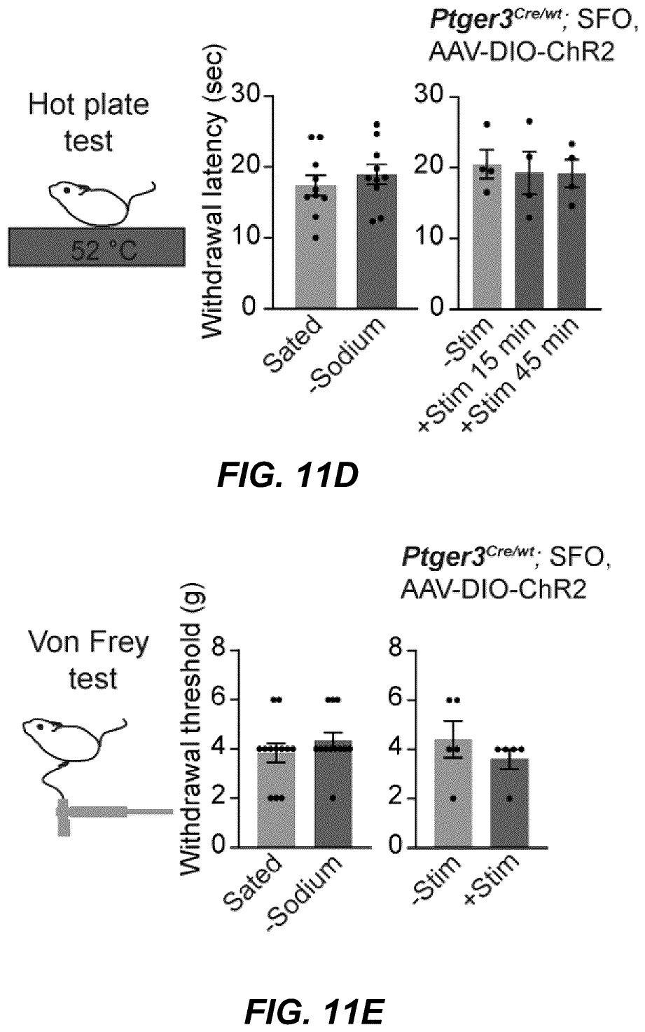

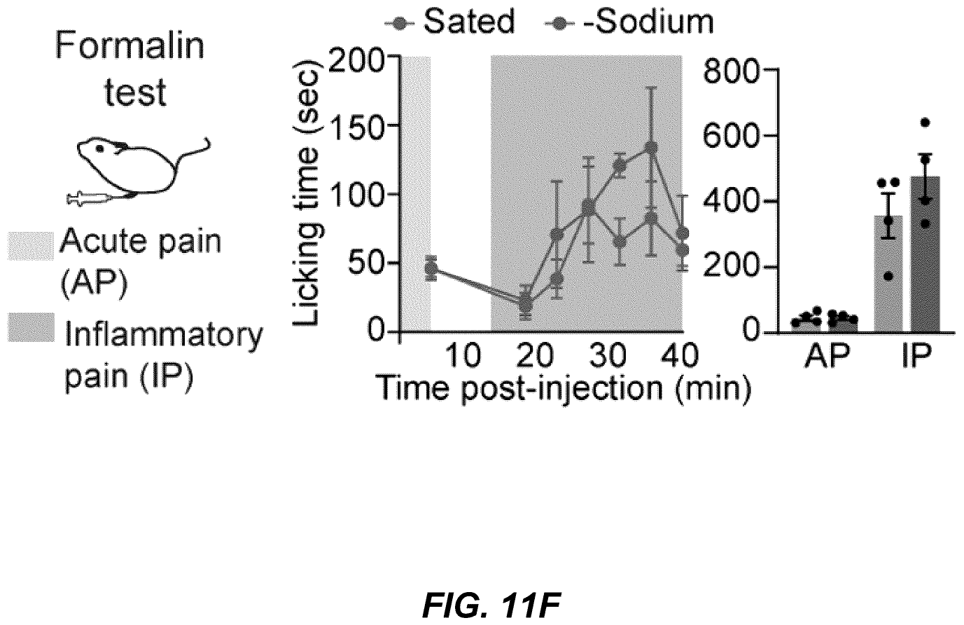

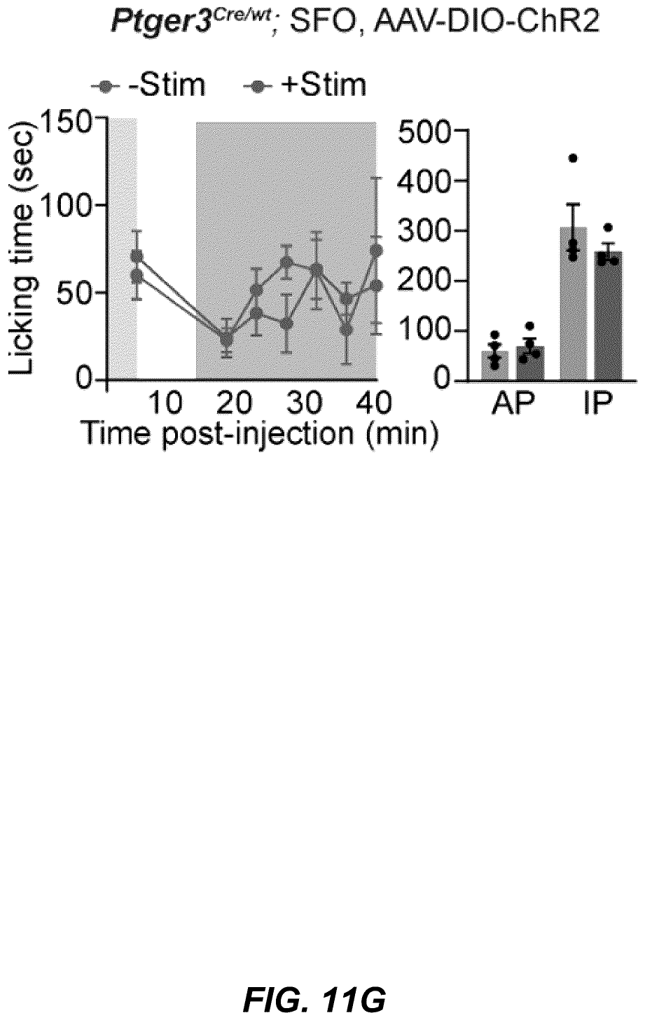

In the mammalian taste system, high salt activates aversive taste pathways, including bitter and sour. Genetic silencing of aversive taste signals (e.g., Trpm5^{-/-}) greatly reduced behavioral avoidance toward salt (FIG. 11A). It was suspected that the activity of Ptger3 neurons might control the sensitivity or saliency of aversive taste signals. If this was true, it was anticipated that the stimulation of SFO^{Ptger3} neurons should reduce behavioral aversion toward bitter or sour taste stimuli. Indeed, while animals exhibited dose-dependent aversion toward bitter and sour tastes, acute stimulation of SFO^{Ptger3} neurons drastically blunted aversive responses to quinine and to a lesser degree, citric acid (FIG. 5A-FIG. 5B). Because acids activate multiple oral sensory pathways, these multimodal signals may have masked the tolerance effects by SFO^{Ptger3} neurons. By contrast, such aversive taste tolerance was not induced by photostimulation of pre-LC^{Pdyn} neurons (FIG. 11B). The behavioral responses to an attractive taste such as sweet was unaffected by the same neural manipulation (FIG. 5C). Moreover, SFO^{Ptger3}-mediated behavioral tolerance was not generalized to conditioned taste aversion or capsaicin, a non-taste oral noxious stimulus (FIG. 11C and FIG. 5D). Taken together, the data demonstrate that behavioral effects through SFO^{Ptger3} neurons are selective toward aversive taste stimuli (FIG. 5E). Consistent with this model, the same neural manipulation had no effect on behavioral tolerance toward other aversive stimuli including heat, acute, and inflammatory pain (FIG. 11D-FIG. 11G).

It was reasoned that if SFO^{Ptger3} neurons encode aversive taste tolerance, artificial stimulation of this neural population should increase general taste palatability. To test this idea, food-deprived animals were given access to sugars (glucose and acesulfame K) with or without an additional bitter component (FIG. 5F). Because sugar and bitter taste mixture is less palatable than the pure sugar solution, mice show reduced preference toward the mix (FIG. 5F and FIG. 5G). However, stimulation of SFO^{Ptger3} neurons suppressed the aversiveness of the mixture, and animals showed increased preference toward the mixture comparable to the pure sugar solution. Similar results were obtained for umami taste (FIG. 5G, right).

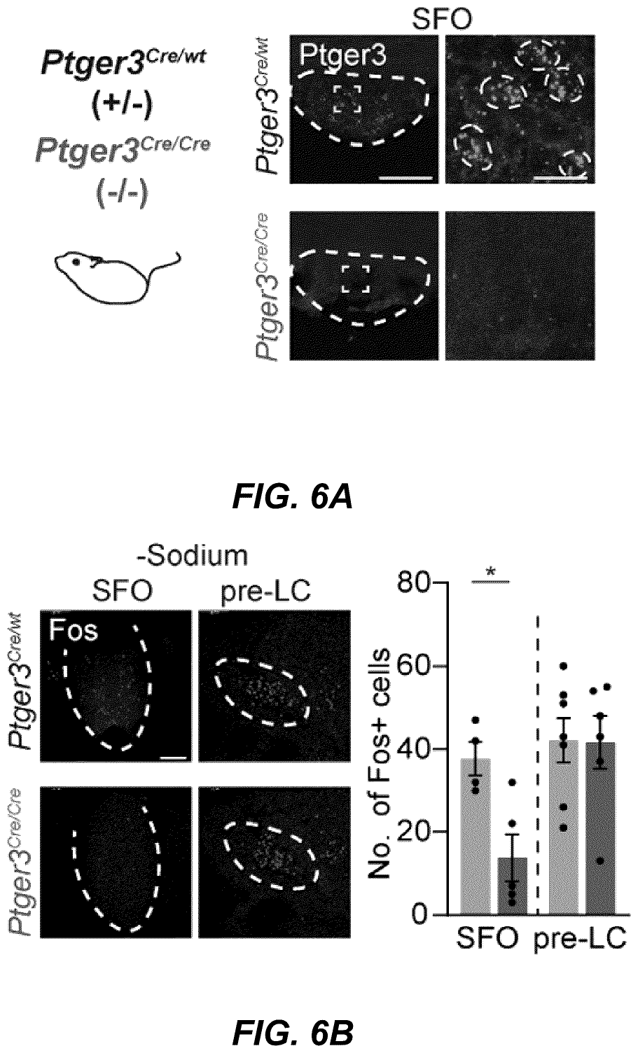

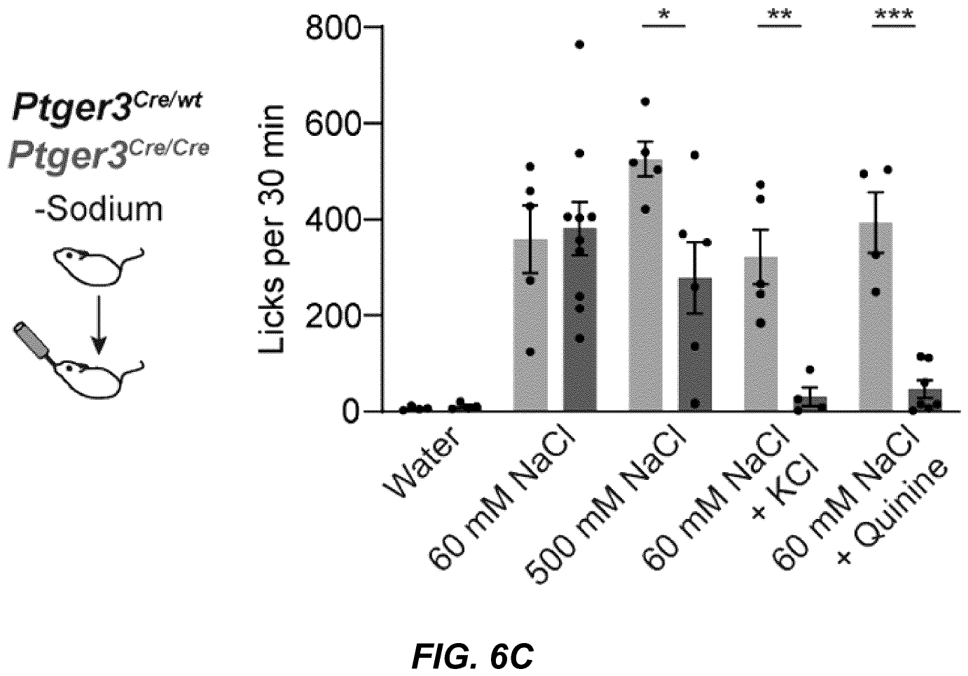

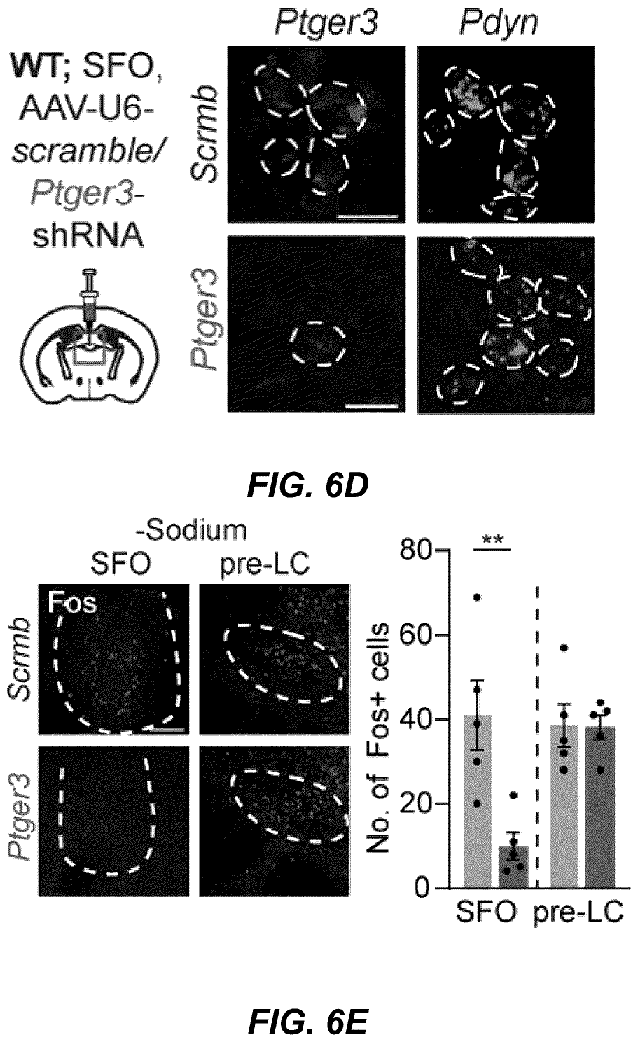

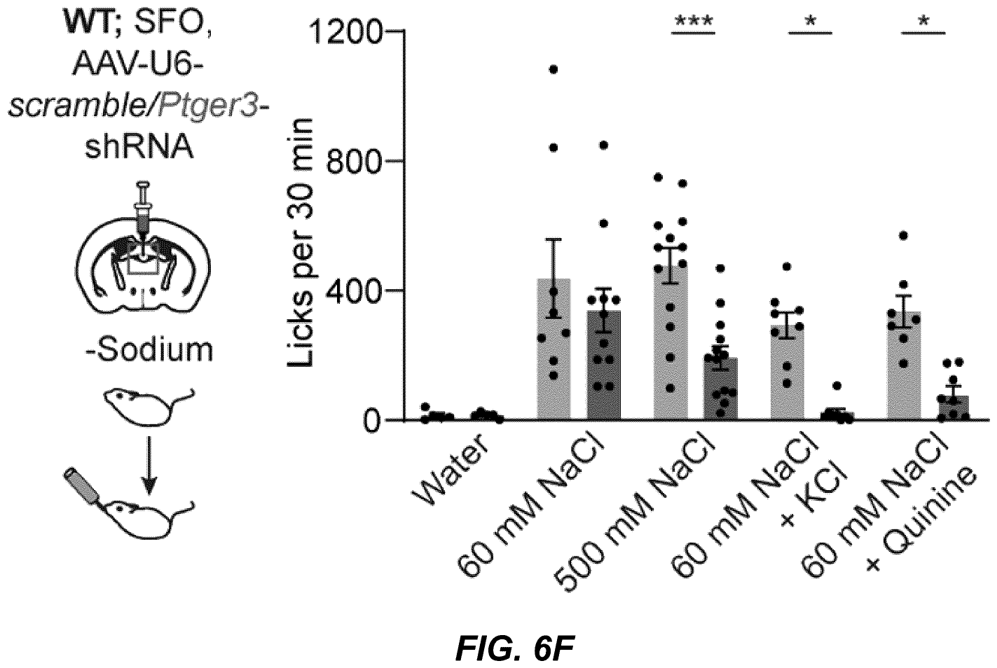

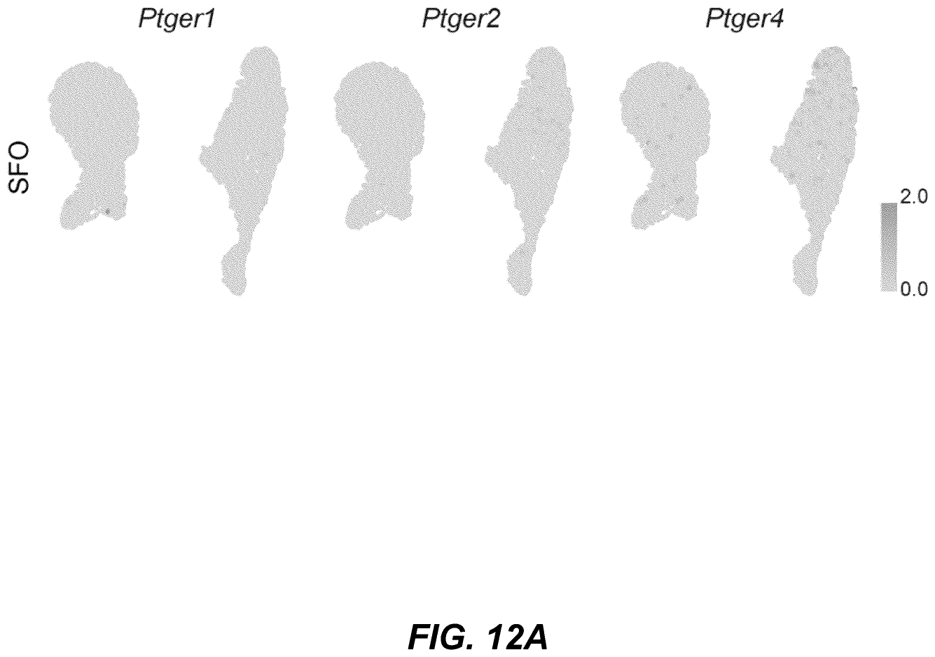

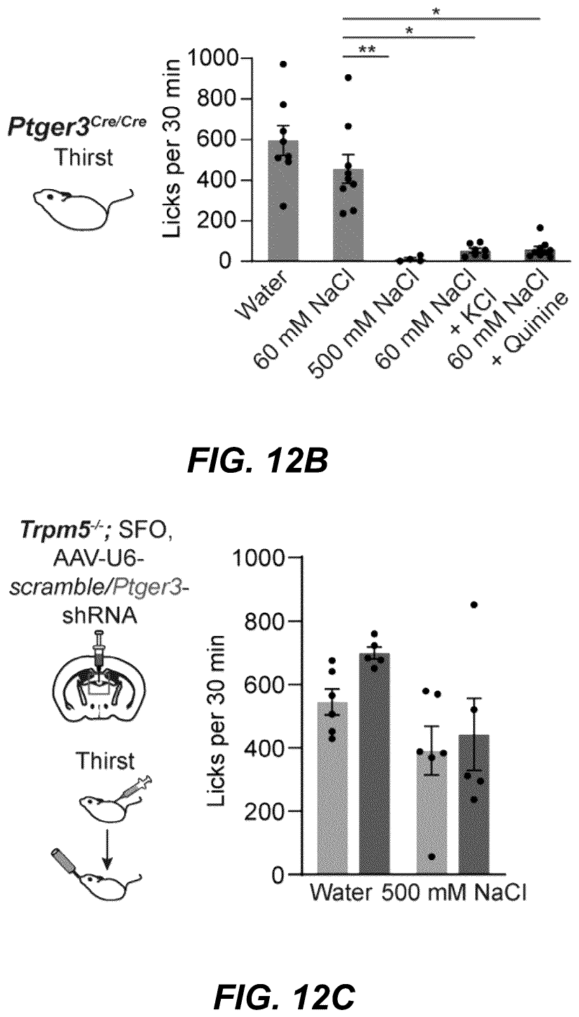

Ptger3 is a receptor for prostaglandins (mainly PGE2), and is involved in various physiological responses. Since Ptger3 is a dominant PGE2 receptor subtype expressed in the SFO (FIG. 12A), it was speculated that PGE2-Ptger3 signaling may be involved in salt taste modulation and ingestion. To test this hypothesis, the role of Ptger3 was examined using a global receptor knockout animal model, Ptger3−/−(or Ptger3^{Cre/Cre} FIG. 6A-FIG. 6C) and local Ptger3 knockdown in the SFO (Ptger3 KD, FIG. 6D-FIG. 6F). Ptger3→mice were obtained as homozygous Ptger3^{Cre/Cre} animals due to premature gene disruption. Deletion of Ptger3 expression was confirmed by in situ hybridization in both cases (FIG. 6A and FIG. 6D). Compared to control animals, Ptger3^{Cre/Cre} and Ptger3 KD significantly reduced Fos immunofluorescence signals under sodium depletion (FIG. 6B and FIG. 6E, SFO), whereas neural activation in the pre-LC was unchanged (FIG. 6B and FIG. 6E, pre-LC). To test the behavioral consequences, salt preference in Ptger3^{Cre/Cre} and Ptger3 KD mice was examined. Consistent with histological results, animals lacking Ptger3 no longer tolerated aversive stimuli including high salt, KCl, and quinine (FIG. 6C, FIG. 6F and FIG. 12B). While Ptger3 is required for salt tolerance, Ptger3 KD in the background of Trpm5−/− did not abolish salt tolerance (FIG. 12C), indicating that behavioral tolerance through Ptger3 requires intact taste signals. Collectively, these data demonstrate that the function of Ptger3 is required for activation of SFO^{Ptger3} neurons and normal salt tolerance under sodium-depleted conditions.

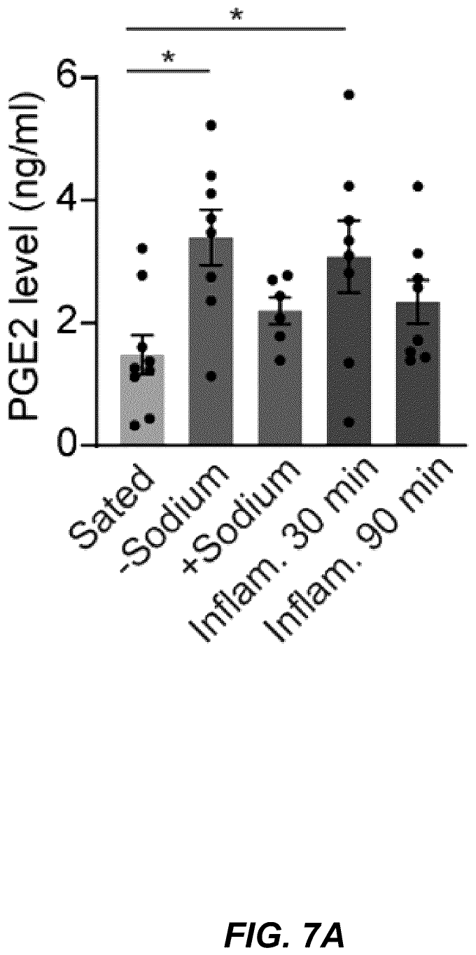

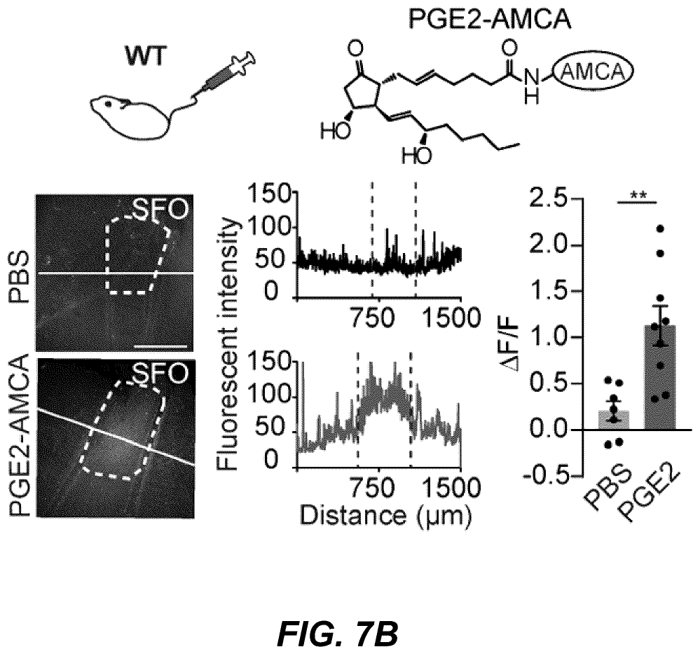

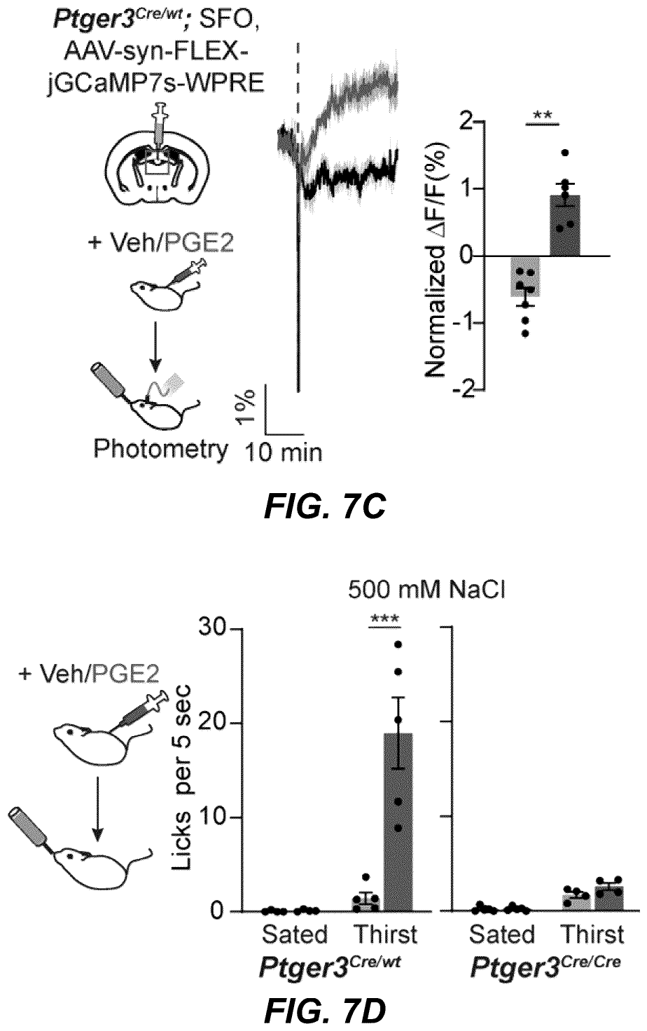

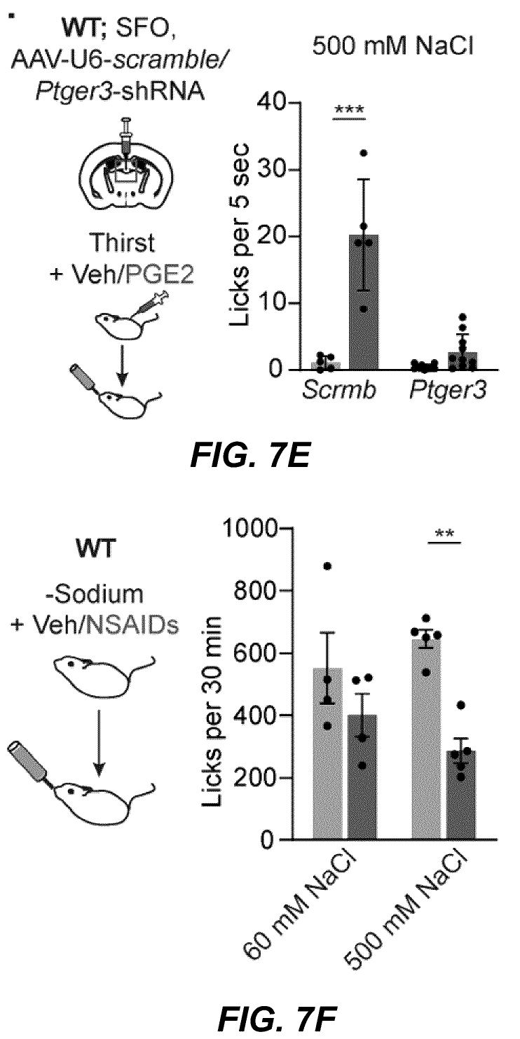

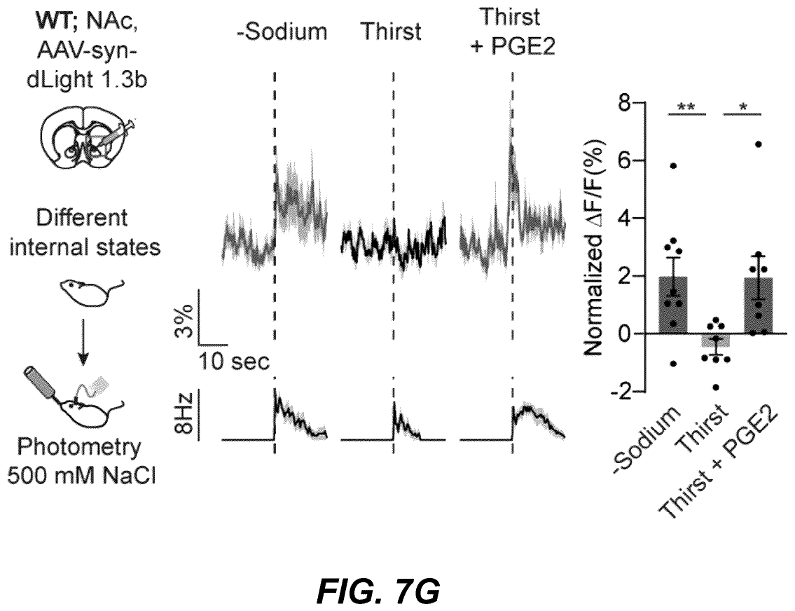

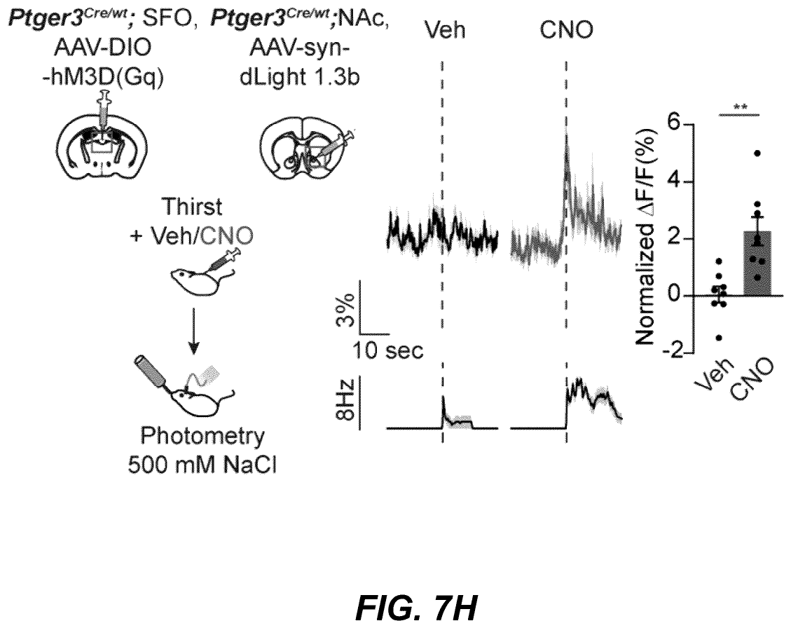

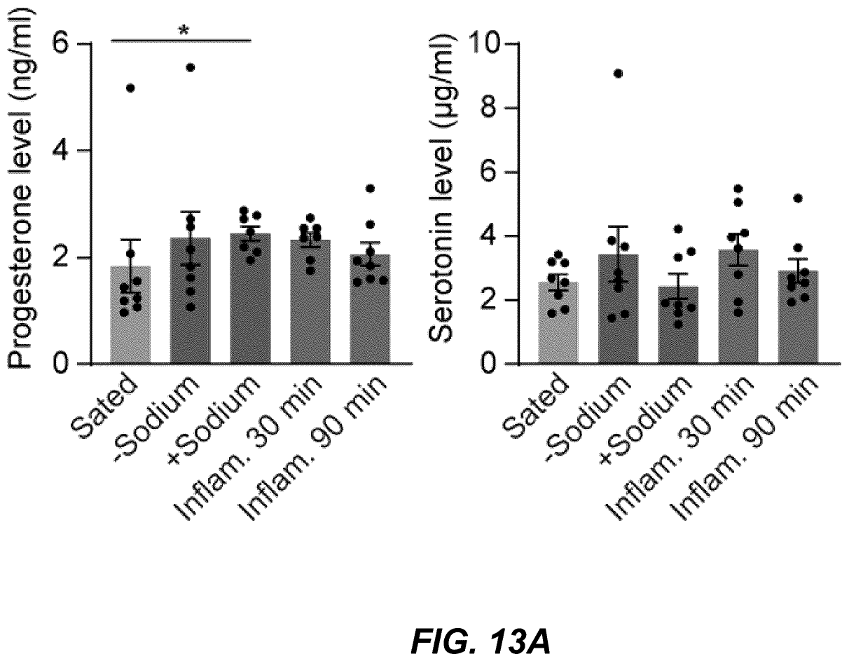

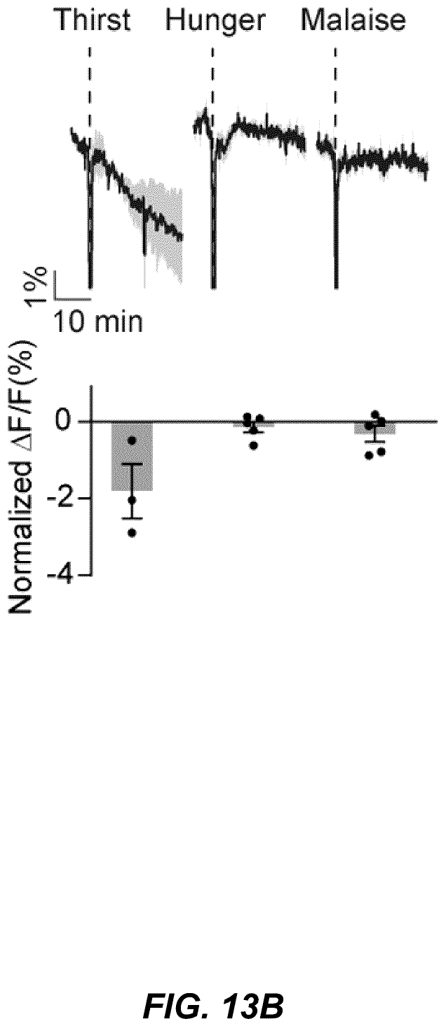

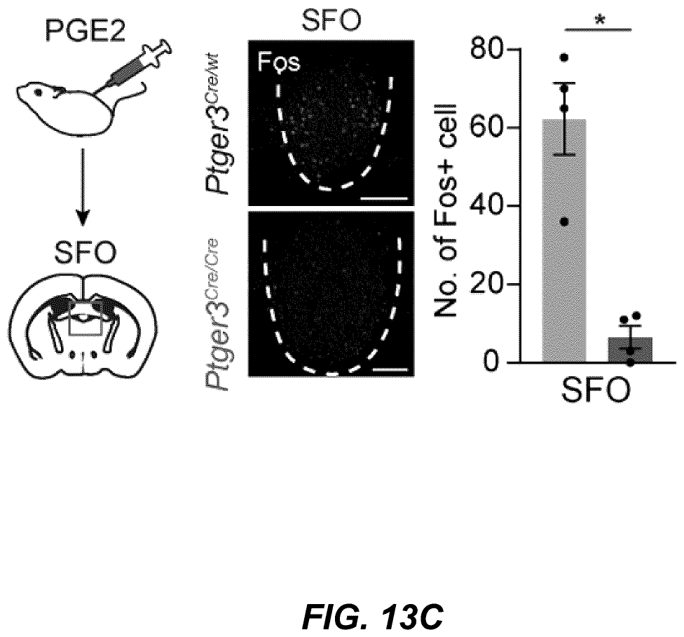

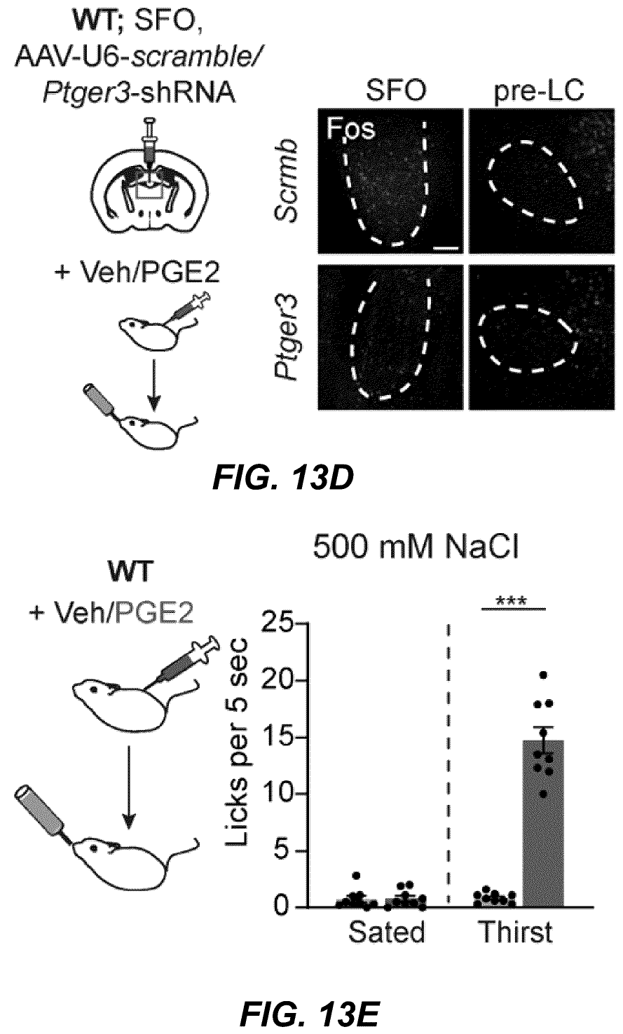

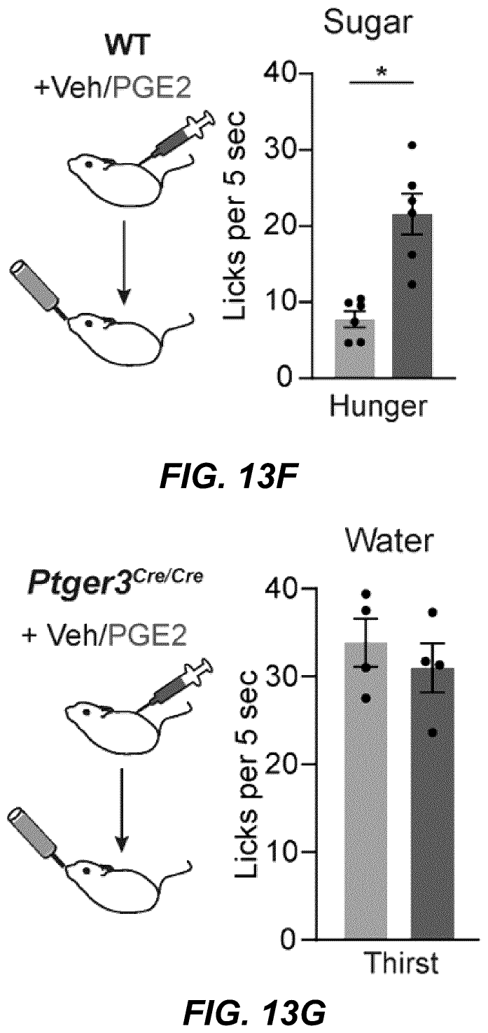

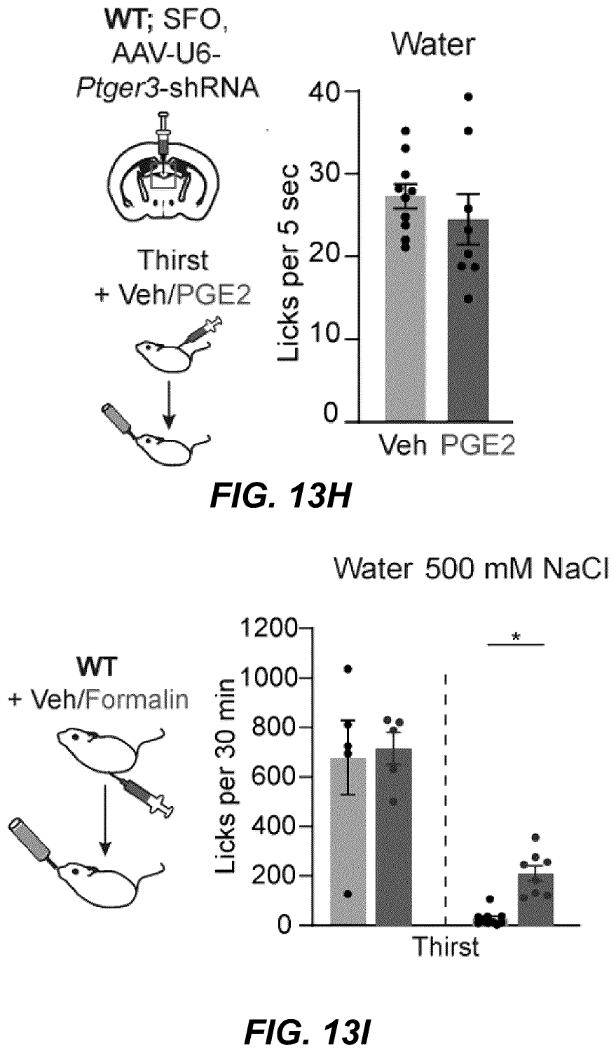

It was next asked whether PGE2 plays a role in salt taste modulation and tolerance. Considering that SFO^{Ptger3} neurons are activated under sodium depletion and inflammation (FIG. 3B), it was tested if sodium depletion elevates PGE2 levels. The ELISA measurement confirmed that circulating PGE2 is significantly increased under sodium depletion, but such elevation was not observed for other inflammation-related molecules such as progesterone or serotonin (FIG. 7A and FIG. 13A). PGE2 conjugated with a small fluorescent dye (PGE2-AMCA) was synthesized to test whether peripheral PGE2 have access to the SFO. Our acute SFO preparation confirmed that fluorescent levels of PGE2-AMCA increased selectively in the SFO within 5 min of peripheral injection (FIG. 7B). This rapid PGE2 penetration is presumably due to the lack of the normal blood brain barrier in the SFO. Moreover, photometry recording from SFO^{Ptger3} neurons that express GCaMP7s (AAV-Flex-GCaMP7s) demonstrated time-locked calcium increases by peripheral PGE2 injections (FIG. 7C). These calcium responses were not observed under osmotic thirst, hunger, or visceral malaise (FIG. 13B). Consistently, strong Fos immunofluorescence signals were found in the SFO by peripheral PGE2 injection in a Ptger3-dependent fashion (FIG. 13C-FIG. 13D).

To directly test tolerance regulation, the effects of PGE2 injection on taste preference were examined. Indeed, like SFO^{Ptger3} neuron stimulation, PGE2-injected wild-type or Ptger3^{Cre/wt} animals exhibited increased salt tolerance under osmotic thirst (FIG. 7D and FIG. 13E) and food-deprived conditions (FIG. 13F), but PGE2 itself did not induce any appetite under sated conditions. Importantly, PGE2-mediated tolerance was largely abolished in Ptger3^{Cre/Cre} animals and Ptger3 KD animals, showing that PGE2-induced high-salt tolerance is mediated through Ptger3 (FIG. 7D-FIG. 7E and FIG. 13G-FIG. 13H). Furthermore, we tested whether prostaglandin production is required for the induced salt tolerance using anti-inflammatory drugs (NSAIDs: ibuprofen and aspirin), a commonly used method to reduce prostaglandin production. It was found that NSAID treatments significantly reduced salt tolerance under sodium depletion (FIG. 7F). Conversely, formalin-induced inflammation increased the PGE2 levels (FIG. 7A) and partially induced high salt tolerance (FIG. 13I). While these studies highlighted the contribution of the PGE2-Ptger3 axis to salt taste modulation, Ptger3 is also activated by other prostaglandin-related molecules. It is feasible that these molecules may play additional roles in sensory modulation under physiological conditions.

Ingestive behavior is intimately linked to the activity of reward circuits. It was tested whether sodium depletion and SFO^{Ptger3} neuron activity alter the activity of dopamine release in the nucleus accumbens (NAc) using genetically encoded biosensor, dLight. We injected AAV-syn-dLight1.3b and an optic fiber into the Nac for real-time optical imaging of dopamine release. It was found that dLight signals rapidly increased when sodium-depleted animals consumed high salt while the same animals under osmotic thirst exhibited minimal activation (FIG. 7G). After subcutaneous PGE2 injection, thirsty mice showed increased high-salt consumption and enhanced dopamine release (FIG. 7G). To test if this effect is recapitulated by the stimulation of SFO^{Ptger3} neurons, we expressed an activation DREADD, hM3Dq, into SFO^{Ptger3} neurons. We then tested dLight signals from the Nac while activating SFO^{Ptger3} neurons by CNO. Compared to vehicle-injected control animals, dopamine signals in the Nac were greatly enhanced during high-salt ingestion only when SFO^{Ptger3} neurons are chemogenetically activated (FIG. 7H). These results further extend the results described herein by showing that the perceived quality of high salt is altered in the presence of salt tolerance signals from the SFO.

TABLE 1
LIST OF GENES IN GLUT1 AND
GLUT2-5 ENRICHED CLUSTERS*

| Glut1 | Glut2-5 |
|---|---|
| 2900055J20Rik | A730036I17Rik |
| 3110035E14Rik | AC124490.1 |
| A830019P07Rik | Ankrd34a |
| Arhgap36 | C130071C03Rik |
| Asic4 | Cacnb2 |
| Bmp3 | Calb2 |
| C1ql1 | Car2 |
| Cd24a | Cdcp1 |
| Cdkn3 | Chrna7 |
| D930028M14Rik | Chst2 |
| Ddr2 | Cntnap5b |
| Erbb4 | Col11a1 |
| Fam19a1 | D930020B18Rik |
| Gm30382 | Dlgap2 |
| Gm43401 | Dpp10 |
| Gpc5 | Dusp6 |
| Gpd1 | Esr1 |
| Hrk | Fam122b |
| Htr7 | Fam163a |
| Ifit1 | Foxp2 |
| Kazald1 | Fzd9 |
| Kcnk9 | Gal |
| Krt16 | Gm11418 |
| Lmo2 | Gm15631 |
| Mapk13 | Gm9885 |
| Neurod1 | Gpr101 |
| Ntn1 | Gpr149 |
| Onecut2 | Gpx3 |
| Onecut3 | Gria1 |
| Pcdh9 | Htr2c |
| Pkib | Id4 |
| Pld5 | Il1rap |
| Ppp1r17 | Il1rapl1 |
| Ptger3 | Il1rapl2 |
| Ptprk | Insm1 |
| Samd11 | Kcnq3 |
| Sncg | Ldb2 |
| Tfap2d | Ltbp1 |
| Tox | Man1c1 |
| Trpc3 | Masp1 |
| Wnt4 | Mirt1 |
| | Mitf |
| | Mpped1 |
| | Ncald |
| | Nhs |
| | Nptx1 |
| | Nrp2 |
| | Ntm |
| | Ntrk3 |
| | Pard3b |
| | Pde1a |
| | Pdzd2 |
| | Penk |
| | Rab15 |
| | Rgs20 |
| | Rimbp2 |
| | Rxfp1 |
| | Sema5a |
| | Sertm1 |
| | Sh3rf1 |
| | Slc12a7 |
| | Slc35f1 |
| | Slc35f3 |
| | Slc35f4 |
| | Slc4a4 |
| | Sorcs1 |
| | Sorcs2 |
| | Spry4 |
| | Stk32c |
| | Svil |
| | Thbs2 |
| | Tmem132d |
| | Tmem163 |
| | Tns3 |
| | Trh |
| | Vsnl1 |
| | Vstm2a |
| | Wipi1 |
| | Wls |

*Genes are listed in alphabetical order

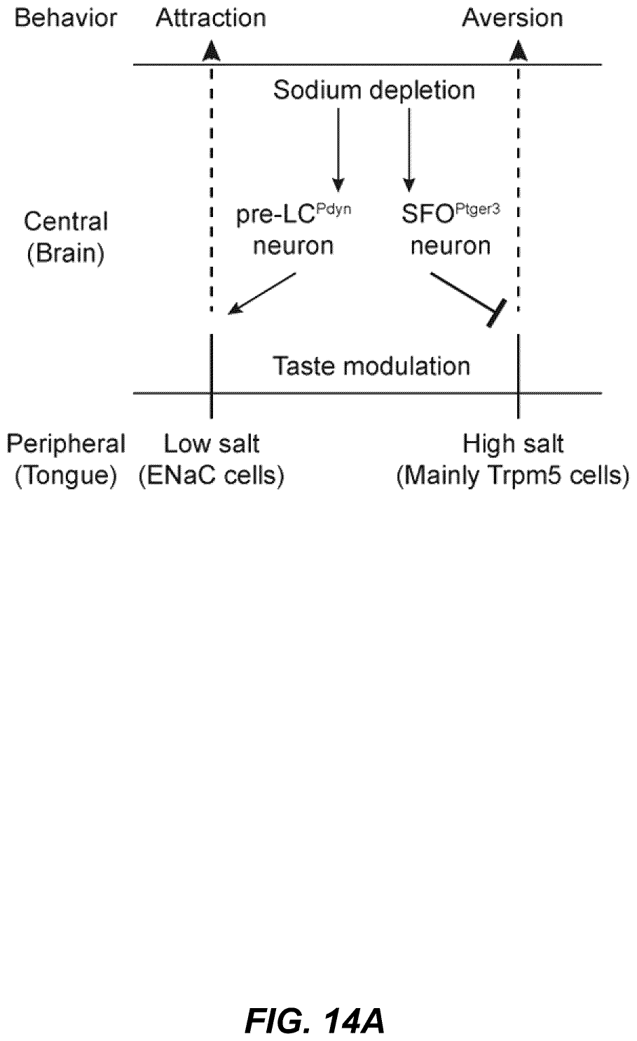

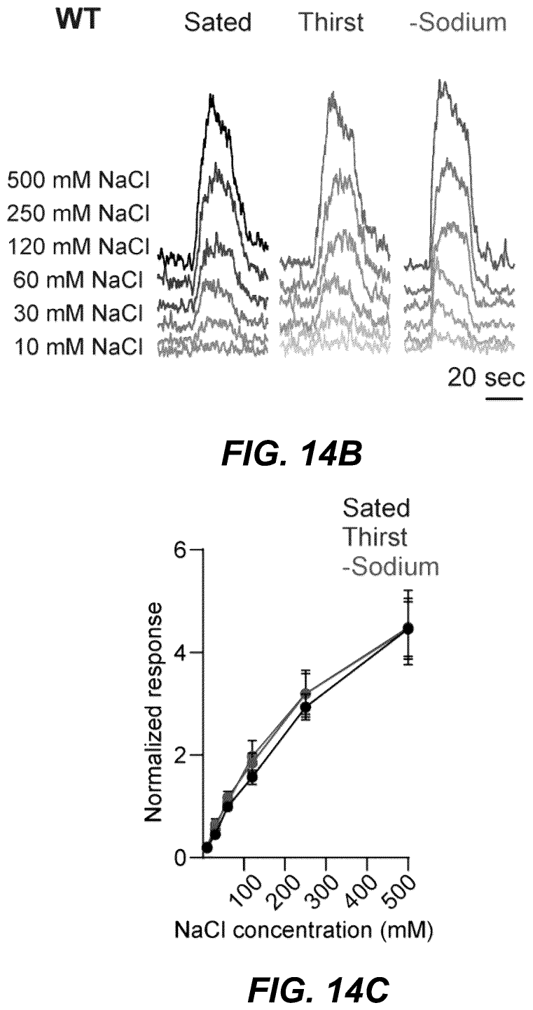

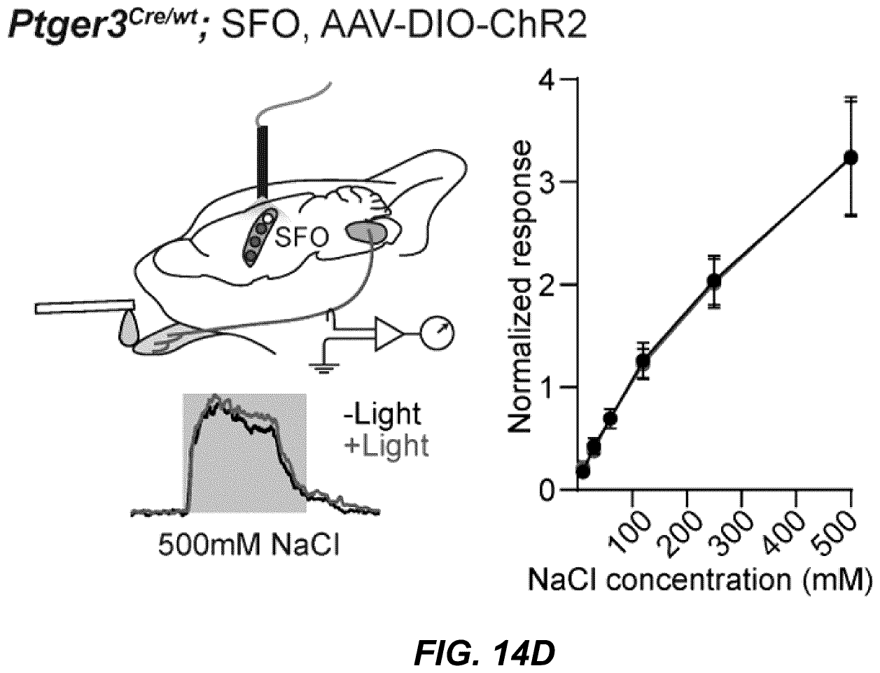

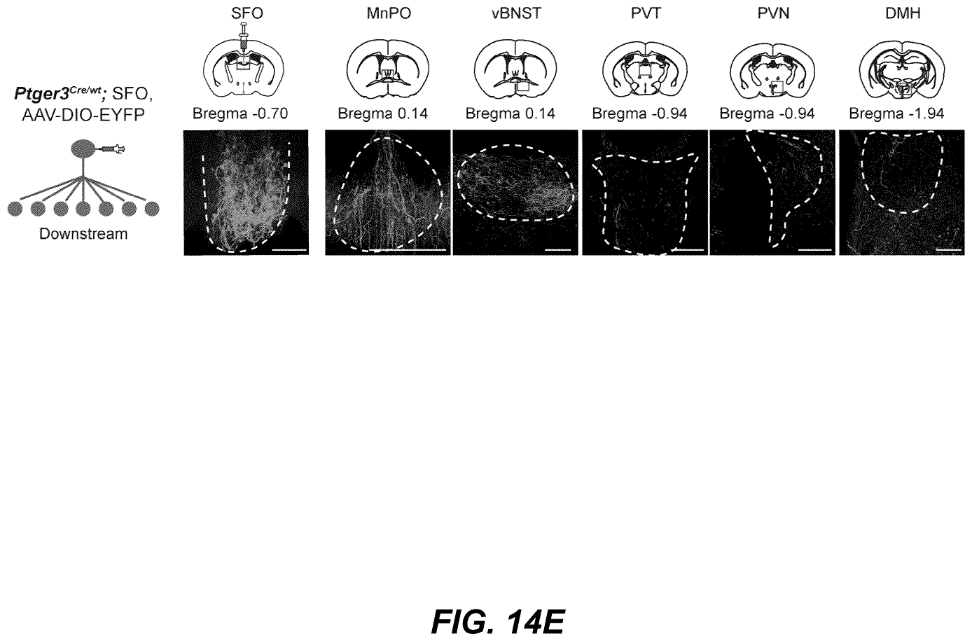

It is provided herein that two critical aspects of salt ingestion, sodium-specific appetitive drive and general salt tolerance are modulated by separate neural populations in an internal-state-specific manner. The described neural perturbation experiments showed that appetitive drive is regulated by the hindbrain neural pathway, while aversion tolerance is regulated by forebrain SFO^{Ptger3} neurons (FIG. 14A). Such a bimodal neural architecture reveals a flexible regulatory mechanism of sodium ingestion by modulating the saliency of sodium taste depending on internal need.

Although sodium depletion suppresses the apparent sensitivity of aversive tastes, such sensory modulation may occur at the peripheral or/and central levels. The results described herein favor the central modulation model, as peripheral taste signals were minimally unaffected by sodium deletion or SFO^{Ptger3} neuron activity. We stimulated the tongue with different concentrations of NaCl while recording peripheral taste nerves using in vivo electrophysiology and observed that the response threshold and amplitude to NaCl were largely unchanged under sated, thirsty, sodium-depleted, or SFO^{Pt}*r-stimulated conditions (FIG. 14B-FIG. 14D). These results suggest that regulation of behavioral sensitivity to aversive taste stimuli is mediated at the central levels.

While the LT is involved in water and sodium balance, how individual LT cell-types are involved in these regulations has been elusive. It is described herein that a genetically defined neuron class that expresses Ptger3 mediates a specific aspect (tolerance) of fluid ingestion. These findings provide key insights into central coding logic of internal water-sodium balance. Under sodium depletion or hypovolemia, SFO^{Ptger3} neurons are activated to promote salt ingestion. Conversely, osmotic thirst activates non-SFO^{Ptger3} LT neurons to drive pure water intake. Thus, each class of LT neurons are activated in an internal-state-dependent manner, yet encodes a specific physiological function (e.g., SFO^{Ptger3} for mineral tolerance). It is currently unclear how SFO^{Pger3} and other LT neurons exert salt tolerance and fluid ingestion through downstream brain areas (FIG. 14E). Future brain-wide downstream analyses will reveal how multiple neuron types regulate water and sodium balance under fluid imbalance.

Brain interoceptive circuits monitor internal states through various molecules. Angiotensin and aldosterone are well-characterized molecules that regulate water and sodium ingestion. This study identified PGE2 as an additional candidate molecular mediator underlying salt tolerance. However, the origin and pathway of PGE2 production awaits further studies. Moreover, since Ptger3 is activated by various ligands besides PGE2, other molecules may also be involved in salt taste modulation. Brain-derived PGE2 is known to regulate core body temperature, while peripheral-derived PGE2 modulates sensory sensitivity, including nociception. Since sodium depletion has no obvious thermoregulatory effects, a source of PGE2 may be from peripheral organs such as the kidney, which leads to activation of SFO^{Ptger3} neurons through Ptger3. Future research will be required to clarify if PGE2 is the only molecule that regulates salt tolerance, and whether other brain areas contribute to this sensory modulation process.

Previous studies have observed elevated tolerance toward aversive stimuli under hunger states. However, the underlying mechanisms appear distinct from salt tolerance. Stimulation of hunger neurons in the arcuate nucleus drives feeding and suppresses various noxious stimuli in parallel through their downstream projections. By contrast, sodium appetite and tolerance are encoded by distinct neural circuits. Why do different interoceptive circuits employ distinct appetitive/tolerance mechanisms? It is well established that the valence of sodium switches in a concentration- and internal-state-dependent fashion. Thus, it is conceivable that the hindbrain pre-LC^{Pdyn} neurons mediate internal-state-specific regulation while forebrain SFO^{Ptger3} neurons encode concentration-dependent regulation toward sodium salts. Such neural architecture would allow interoceptive and exteroceptive signal integration to optimize sodium consumption.

Unlike non-caloric artificial sweeteners such as sugar substitutes, sodium is the only safe cation that exhibits salty taste. Even though salt overconsumption is a significant health risk factor, modulation of salt consumption has been challenging due to the lack of sodium substitutes. The findings described herein shed light on a potential strategy to modulate salt taste by saliency control. Described herein is that activation and inhibition of SFO^{Ptger3} neurons bidirectionally regulated sodium tolerance and consumption (FIG. 3D-FIG. 3F). The SFO lacks the normal blood-brain barrier, making it accessible to compounds in systemic blood circulation such as PGE2. Thus, identifying compounds that activate or inhibit LT^{Ptger3} neurons may provide a salt-taste-modifying strategy to control sodium consumption.

### Methods

### Animals

All procedures followed the US NIH guidance for the care and use of laboratory animals and were approved by the California Institute of Technology Institutional Animal Care and Use Committee (protocol: 1694-14). Male and Female mice at least 8 weeks were used for behavioral tests and histology characterization. No sex difference is observed in this study. For scRNA-seq experiments, 7-8-week-old C57BL/6J mice were used. C57BL/6J were purchased (000664) from the Jackson Laboratory. Pdyn^{Cre} mice were a gift from B. Lowell and M. Krashes (Jackson lab, 027958). Heterozygous Pdyn^{Cre} mice were used for all experiments. Trpm5−/− mice were generously provided by C. Zuker (Jackson lab, 013068). Mice were housed in temperature-controlled and humidity-controlled rooms with a 13:11 hr light:dark cycle. Mice are provided with ad libitum access to chow and water unless mentioned in the nutrient deprivation experiments.

### Generation of the Ptger3Cre Mouse Line

Ptger3^{Cre} animals were generated by inserting mnCre:GFP cassette just 5′ of the initiation codon of Ptger3 gene and deleting the first 40 amino acids. The cassette had a Myc-tag and nuclear localization signals at the N-terminus. The targeting construct was electroporated into G4 ES cells (C57Bl/6×129 Sv hybrid) and correct targeting was identified by Southern blot. The frt-flanked SV-Neo gene was removed via breeding with Gt(Rosa)26Sor-FLP recombinase mice and then backcrossing to C57BL/6J mice for >6 generations. Homozygous Ptger3^{Cre/Cre} mice are equivalent to Ptger3-because of the amino acid deletion.

### Viral Constructs

The following viruses were used: AAV5-EF1a-DIO-ChR2-EYFP, 3.0×10¹³ viral genomes per ml. AAV2-EF1a-DIO-ChR2-mCherry, 5.1×10¹² viral genome copies per ml. AAV2-EF1a-DIO-mCherry, 4.6×10¹² genome copies per ml. AAV8-hSyn-DIO-hM4D(Gi)-mCherry, 1.9×10¹³ viral genomes per ml. AAV2-hSyn-DIO-hM3D(Gq)-mCherry, 6.5×10¹² viral genomes per ml. AAV2-Flex-taCaso3-Tevp, 1.8×10¹² viral genome copies per ml. AAV5-Flex-taCasp3-Tevp, 4.2×10¹² viral genome copies per ml. AAV9-syn-dLight1.3b, 2.6×10¹³ viral genomes per ml. AAV1-syn-FLEX-jGCaMP7s-WPRE, 1.9×10¹³ viral genomes per ml. AAV9-GFP-U6-scrmb-shRNA, 3×10¹³ viral genomes per ml. AAV9-GFP-U6-m-Ptger3-shRNA (shAAV-269740), 1.5×10¹³ viral genomes per ml.

### Method Details

### Surgery.

Surgery procedures were performed according to methods known in the art. Briefly, ketamine (1 mg/ml) and xylazine (10 mg/ml) in saline were injected intraperitoneally at a dose of 10 ml per kg body weight (BW) for anaesthesia. Ketoprofen was injected subcutaneously at a dose of 5 ml per kg BW. Surgery was performed with a stereotaxic apparatus (Narishige, SR-5M-HT) on a heating pad at 37° C. Virus was delivered with a microprocessor-controlled injection system (World Precision Instruments, Nanolitre 2000) at 100 nl/min. The following coordinates were used: for pre-LC: anterior-posterior (AP), 9,000, medial-lateral (ML), 1,000, ventral-dorsal (VD), 3,800; for SFO: AP, 4,000, ML, 0, VD, 2,500; for OVLT: AP, 2,475, ML, 0, VD, 4,750; for dorsal part of the NAc medial shell: AP, 2,100, ML, 700, VD, 4,000. For optogenetic experiments, virus was delivered unilaterally at a volume of 120-200 nl. Implants were made by gluing a 200-μm fibre bundle (Thorlabs, FT200EMT) to a ceramic ferrule (Thorlabs, CF230-10) with epoxy. The fibre implant was placed 200 μm above the virus injection site. For fiber photometry experiments, virus was delivered at a volume of 150-200 nl (calcium measurement) or 500 nl (dopamine measurement) per region. A 400-μm fiber bundle (Thorlabs, FT400UMT) was used instead. For chemogenetic inhibition experiments, virus was delivered at 180-200 nl per region. For ablation experiments, virus was delivered at 200-250 nl per region. For local knockdown experiments, virus was delivered at 150-200 nl per region. At the end of experiments, all animals were euthanized for histological examination.

### Internal State Induction

Sodium depletion: Mice were injected intraperitoneally with furosemide at a dose of 50 mg per kg BW and maintained with a low-sodium diet for 24 hr before behavioral assays or immunohistological staining.

Sodium repletion: Mice were injected intraperitoneally with furosemide at a dose of 50 mg per kg BW 24 hr before the experiment and maintained on a normal diet.

Osmotic thirst: Mice were injected intraperitoneally with either 2 M NaCl (5 ml per kg BW) or mannitol solution (10 ml per kg BW) and maintained with no food or water for 10 min before the behavioral assay or 1 hr before immunohistological staining. Mannitol was used for long-term consumption assay and Fos immunostaining, and NaCl was used for short-term consumption assay. No significant differences were found between the results obtained with these two solutions.

Sodium depletion with osmotic thirst: Mice were sodium depleted via furosemide as mentioned above. Mice were injected intraperitoneally with 2 M mannitol solution (10 mg per kg BW) and maintained with no food or water for 10 min before behavioral assay.

Hunger: Mice were food deprived for 24 hr before the behavior test or immunohistological staining.

Hypovolemic thirst: Mice were injected intraperitoneally with furosemide at a dose of 50 mg per kg BW and maintained with no food or water for 3 hr before behavior test or 4 hr immunohistological staining.

PGE2-induced behavior: PGE2 was injected subcutaneously at a dose of 1 mg per kg BW. Animals had no food or water for 15-90 min before behavioral experiments. For immunohistochemistry, PGE2 was injected at a dose of 3 mg per kg BW and maintained with no food or water for 60 min before euthanasia.

Visceral malaise: Mice were injected intraperitoneally with 0.15 M LiCl at a dose of 15 ml per kg BW and euthanized 1 hr after injection for immunohistological staining.

### Solution Preparation

Tastants: We chose the concentrations of taste/salt solutions based on previous literature. In the mammalian taste system, aversive components of high salt and KCl share the same taste pathways. Thus, we supplemented KCl in low-salt solutions to increase the aversiveness of salt solution without changing activity of the low-salt specific ENaC pathway.

Capsaicin: Capsaicin was prepared as 3 mM stock in 30% ethanol. For brief access assay, the solution was further diluted to 1 or 0.3 M with water. Water containing ethanol equal to 1 M capsaicin was used for control.

Prostaglandin: PGE2 powder was diluted in DMSO at 50 mg/ml as stock. For behavioral assay, we used the final concentration of 1 mg per kg BW in PBS containing 1.7% DMSO (v/v). The same vehicle with DMSO was used as control.

### Behavioral Assays

All assays were performed in a custom gustometer (Dialog Instruments). All animals were at least trained with osmotic thirst and sodium depletion prior to the behavior trials. Animals were trained to drink at least 200 licks for 500 mM NaCl in 30 min under sodium depletion. Generally, two training sessions with sodium depletion are sufficient to achieve this criterion. Animals often show neophobia to a new solution even if it was an attractive stimulus. To prevent this, all tastants at lower concentrations were exposed to animals before experiment. For tolerance tests, one-bottle consumption assay was used in order to avoid preference development during the session.

For 30-min access assay, mice were presented with 1 bottle for 30 min and consumption was measured.

For 5-sec brief access assay, mice were presented with 1 bottle of solution for 30 sec per trial for maximum waiting time before the first lick, and 40 sec between trials. After an initial lick, animals were allowed to drink the solution for additional 5 sec before a shutter closed. To avoid taste conditioning effect, animals were tested with only one solution per day. After testing aversive solutions, bitter and high salt, animals sometimes refused to drink any solution. These animals were retrained with water and low salt until their behavioral level recovers to our criteria stated above.

### Optogenetic and Chemogenetic Manipulation

For optogenetic activation, 473-nm laser-pulse sequences were delivered via an optic cable (Doric Lenses, MFP—FC-ZF) with pulse generators (World Precision Instruments, SYS-A310 or Quantum composers, Sapphire 9200). The laser intensity was maintained at 10 mW at the tip of the fiber. For 30-min access assay, the light stimulation was given at 20 Hz with a 1-see on 3-sec off paradigm. For 5-sec brief access assay, the light stimulation was given at 20 Hz throughout the trial except the paradigm in FIG. 3C.

For chemogenetic experiments, clozapine N-oxide (CNO) was administered intraperitoneally at doses of 10 mg per kg BW for inhibition or 1 mg per kg BW for activation 20 min before the consumption trial started. Pure water instead of saline was used as vehicle to avoid sodium repletion.

### Long Term Consumption Assay

Liquid consumption level was monitored in the Biodaq system (Research Diets). Mice were individually housed and acclimated in the cage at least for 24 hr before the data collection. We considered that animals were acclimated when they consumed more than 1 g of water and low salt overnight. Animals were offered only one type of liquid overnight and were provided with water during the daytime to replete. 60 mM NaCl, 60 mM NaCl with 440 mM KCl, 60 mM NaCl with 40 mM CaCl₂, 60 mM NaCl with 40 mM MgCl₂, 500 mM NaCl and water were applied. Data for consumption were analyzed from 6 to 9 pm. Prolonged measurement with aversive solutions often causes dehydration if animals refuse to drink. To avoid internal state transition over time, we measured consumption within a limited time window.

### Pain Induction

Acute Pain (foot shock): Animals were acclimatized in the operant conditioning chamber for 10 min (MedAssociates). A constant current of 0.25 mA was delivered to the metal grid floor. Animals moved freely in the setting. The shock was delivered for a continuous 5-sec window followed by a 10-sec rest. Each trial lasted 2 min. For immunohistological staining, mice were euthanized 1 hr after exposure. The light stimulation was given at 20 Hz with a 1-sec on 3-sec off paradigm.

Thermal pain (heat shock): A hotplate (Columbus instruments, 93291) was maintained at 52° C. Animals were placed onto the hotplate and the latency for licking paws was recorded. To test the effect of optogenetic stimulation, animals were tested on the hotplate, and re-tested following stimulations of 15 and 45 min. The light stimulation was given at 20 Hz with a 1-sec on 3-sec off paradigm.

Mechanical threshold (von Frey Test): Mice were habituated on a metal grid floor, and were tested with von Frey filaments (Bioseb, BIO-VF-M) in ascending order. Each filament was tested 5 times. The withdrawal threshold was determined when the mice withdrew the paw for more than 3 times as the filament was applied until bent. To test the effect of optogenetic stimulation, mice were tested before stimulation and following a stimulation of 60 min. The light stimulation was given at 20 Hz with a 1-sec on 3-sec off paradigm.

Inflammatory pain (formalin test): Mice were injected with 2% formalin (20 l) in the dorsal hindpaw and placed in behavior chamber. Videos were taken for 1 hr following the injection and analyzed for the time mice spent licking the dorsal hindpaw. Mice with more than 30-sec licking paw time in the first 5 min after injection were used for video analysis. Each mouse was subjected to formalin injection twice: in one hind paw under sated conditions, and another under either sodium depleted or photostimulated conditions. To test the effect of optogenetic stimulation, light stimulation was given at 20 Hz with a 1-sec on 3-sec off paradigm for 60 min. For ELISA assay, blood was collected 30 or 90 min after formalin injection.

### Taste Aversion

Animals accessed 2 mM AceK in the gustometer after 24 hr food deprivation. Within 5 min after the trials finished, animals were given 0.15 M LiCl intraperitoneally, at a dose of 15 ml per kg BW. After one day rest, animals were food deprived again for 24 hr, and exposed to 2 mM AceK in the gustometer. Photostimulation was given alternatively in 10 trials. Lick number was quantified respectively for the light stimulation on and off sessions.

### Inflammation and Consumption

Formalin-induced tolerance: Mice were injected with formalin or PBS in the dorsal hindpaw 45-60 min prior to the lick access. Osmotic thirst was induced 10-15 min prior to the lick access. Lick number was recorded for 30 min.

NSAIDs-induced loss-of-tolerance: NSAIDs were used to suppress PGE2 production. Mice were given water supplemented with ibuprofen (1 mg per ml) for three days. On the second day, mice were injected with furosemide to induce sodium depletion. On the third day, aspirin (40 mg per kg BW) was injected intraperitoneally 1-2 hr prior to the lick access. Lick number was recorded for 30 min.

### Fiber Photometry

Calcium measurement from SFO^{Ptger3} neurons: Mice were headfixed to eliminate the interference from movement. Photometry recording was performed using a commercial photometry system (Neurophotometrics, FP3002). Two LED of different wavelength (470 nm and 415 nm) were bandpass filtered and a patch cord (0.48 NA, Doric lenses) was attached to the photometry system via a 20× objective. The sampling rate was fixed at 40 Hz. Data were acquired with Bonsai (Neurophotometrics) by drawing a region of interest and calculating the mean pixel value, and then were exported to MATLAB for further analysis. The isosbestic signal (415 nm) was fit with a biexponential model and was then linearly scaled to the calcium signal (470 nm). The ΔF/F was calculated as (raw 470-nm signal−fitted 415-nm signal)/(fitted 415-nm signal). For all sessions, stimulus time was recorded through a customized TTL button. During PGE2 activation experiment (FIG. 7C), mice were given PGE2 at a dose of 1 mg per kg BW or DMSO in 100 μl PBS subcutaneously. During other internal-state treatments, mice were exposed to the following stimuli via intraperitoneal injection: 2M mannitol (10 ml per kg BW); 10 g of ghrelin (in 100 μl PBS), and 0.15M LiCl (15 ml per kg BW). All analysis used the average of 5 min prior to the stimulus as a baseline mean. Normalized ΔF/F was calculated by subtracting the baseline ΔF/F from the mean ΔF/F between 100 and 1500 sec after the stimulus.

Dopamine measurement: Mice were freely moving in the chamber with gustometer. Bulk fluorescence signals were collected with a fiber photometry setting as described with the light of 405 nm and 490 nm. The ΔF/F was calculated as (raw 490-nm signal−fitted 405-nm signal)/(fitted 405-nm signal). Mice were induced with osmotic thirst 10-15 min, or sodium depletion 24 hr before the access of 500 mM NaCl. For wild-type mice, under PGE2 activation condition, mice were injected PGE2 at a dose of 1 mg per kg BW subcutaneously 1 hr before the shutter open. For Ptger3cre mice, mice were injected with either CNO or vehicle 20 min before the shutter opened. Lick number was recorded simultaneously with the fluorescence signal collection. The first lick bout was identified with the lick rate more than 3.5 and lick number more than 5, with less than 1-sec interval to the subsequent lick. If no lick bout was detectable (observed with osmotic thirst), any first lick event was included into the data. All baseline signals were calculated by averaging signals between 10-20 sec prior to the first lick. Response amplitude (normalized ΔF/F) was calculated by subtracting the baseline ΔF/F from the mean ΔF/F between 0 and 10 sec after the lick initiated.

### PGE2-AMCA Dye Infusion

PGE2 was conjugated to AMCA as a synthesized dye. Mice were anaesthetized with ketamine (1 mg/ml) and xylazine (10 mg/ml) at a dose of 10 ml per kg BW. Mice was administrated with the dye or vehicle via tail vein at a dose of 1 mg per kg BW. Five minutes post-injection, fresh brain was extracted, and SFO was dissected under a microscope, Several drops of PBS was added onto the slice to prevent tissue dryness. Slices were imaged under LSM 880 with Fast Airyscan using 20× z-stack (Airyscan SR Mode, detector-1.25 AU). SFO was identifiable between two paralleled blood vessels. For analysis in ImageJ, the image was rotated to an angle that the two blood vessels were vertical. Fluorescence was extraced by an arbitrary horizontal line at the dorsal side. The ΔF/F was calculated as (fluorescence from 100-μm in SFO−average of fluorescence from dorsal and ventral 100-μm outside of SFO)/(average of fluorescence from dorsal and ventral 100-μm outside of SFO).

### Immunohistochemistry

Mice were euthanized with CO₂ and perfused with PBS and 4% paraformaldehyde. Extracted mouse brains were fixed overnight at 4° C. in 4% PFA and sectioned coronally to 100-μm slides via vibratome (Leica, VT-1000 S). The sections were blocked (10% donkey serum, 0.2% Triton X-100 in PBS) for 1 hr at room temperature and incubated with primary antibody at 4° C. overnight. The following primary antibodies were used: rabbit anti-FOS (1:500), sheep anti-Foxp2 (1:2,000), chicken anti-GFP (1:3,000), rat anti-mCherry (1:500), and goat anti-nNos (1:500). The sections were washed three times with PBS and incubated with the secondary antibody (1:500; Jackson Immunoresearch) and DAPI (2 g/ml) for 4 hr under room temperature. After three times wash with PBS, the sections were mounted on the glass slide. All slides were imaged with confocal microscope (Leica, TCS SP8) or slide scanner (Olympus, BX61VS).

### Plasma PGE2, Progesterone, and Serotonin Concentration Measurement

Mice were anaesthetized with isoflurane. Blood was collected via heart puncture into EDTA-coated tubes (BD Microtainer, 365974) and kept on ice. Plasma was then separated by centrifugation at 1,500 g for 20 min. ELISA assays were performed following the protocols of PGE2 ELISA kit (Cayman Chemical, 514531), serotonin ELISA kit (Eagle Bio, SER39-KO1), and progesterone ELISA kit (Cayman Chemical, 582601).

### Taste Nerve Recording

Surgery and recording procedures were performed as described. Briefly, mice were anesthetized with pentobarbital (100 mg per kg BW) and placed in a custom-made head-fixation setup, with body temperature maintained at 37° C. Tracheotomy was performed. The right branch of the chorda tympani nerve was exposed, and recording was performed with a high-impedance tungsten electrode hooking the nerve bundle. Tastant stimuli were delivered with a pressurized perfusion system (AutoMate Scientific) for 20 sec, with a 40-sec wash with artificial saliva. The artificial saliva was made with 20× stock and on the day of experiment, was diluted with the addition of 6 mM KHCO₃ and 6 mM NaHCO₃. The pH was adjusted to 7.2-7.6. For recording, each mouse underwent two sets of experiments in a specific internal state. Two sets of experiments were in a randomized order and each experiment was repeated with 2-3 trials according to the mice condition. For the NaCl experiment, 10/30/60/120/250/500 mM NaCl and 4 mM AceK were applied. For multiple tastant experiments, the following taste stimuli were used: 60 mM NaCl (salty), 10 mM citric acid (sour), 4 mM acesulfame potassium (sweet), 50 mM monopotassium glutamate plus 1 mM inosine monophosphate (umami), 500 mM KCl (bitter), and 1 mM quinine (bitter). Signals were recorded with Axon Digidata 1550B Low-Noise Data Acquisition System, and Grass RPS312 RM Power Supply with P511 AC Amplifier (Natus Neurology).

The data were processed using a custom Matlab code. Briefly, the original data (sampled at 10 k Hz) were time-binned at 0.1 sec. The recording traces were visualized by processing time-binned data with a rolling-median of 3 sec. A 3-sec time window before each stimulus served as a baseline. A 30-sec response window was quantified, starting when the signals exceeded 3 standard deviations (SD) above baseline. The data were normalized to the average of two consecutive 4 mM AceK trials. For photostimulation trials, when the tastants washed through, light was delivered at 20 Hz, 10 mW for 30 sec.

### Single Cell RNA Sequencing

Tissue processing into single-cell suspensions: For the sodium-depleted condition, 15-20 mice, 7-8 weeks old, were sodium depleted for 24 hr. For sated condition, 15 sated mice were used. The tissue processing procedure was performed as described. Briefly, upon isoflurane anaesthesia, the brains were exacted from the mice and kept in ice-cold carbogenated NMDG-HEPES-ACSF. Brains were sectioned to 2 mm, SFO was harvested into ice-cold NMDG-HEPES-ACSF by peeling the tissue (SFO) microdissection under microscope. After tissue collection, NMDG-HEPES-ACSF was replaced with trehalose-HEPES-ACSF containing papain (80 U/ml; pre-activated with 2.5 mM cysteine and a 30-min incubation at 34° C. with 0.5 mM EDTA) and 15 M actinomycin D. Extracted SFO tissue was incubated at 34° C. with gentle carbogenation for 60-90 min. The tissue was pipetted periodically every 10 min during this digestion. At the end of enzymatic digestion, the medium was replaced with 200 μl of trehalose-HEPES-ACSF with 3 mg/ml ovomucoid inhibitor 25 U/ml DNase I and 15 M actinomycin D. The SFO tissue was gently triturated with 600, 300 and 150 μm-diameter fire-polished glass Pasteur pipettes in less than 10 mins. The tissue was processed into a uniform single-cell suspension and the total volume was brought up to 1 ml with trehalose-HEPES-ACSF with 3 mg/ml ovomucoid inhibitor. The suspension was filtered through a 40-μm cell strainer (Falcon, 352340) into a new microcentrifuge tube and was centrifuged with 300 g for 5 min at 4° C. The supernatant was discarded, and the cell pellet was resuspended with fresh ice-cold resuspension buffer and kept on ice while cell densities were quantified with a hemocytometer. The final cell suspension volume estimated to retrieve ˜10,000 single-cell transcriptomes were added to the 10× Genomics RT reaction mix and loaded to the 10×Single Cell G chip (10× Genomics, PN-1000127). The Chromium Single Cell 3′ GEM, Library and Gel Bead Kit v3.1 (PN-1000128) and the Single Index Kit T Set A (PN-1000213) were used. The cDNA and library amplification underwent 11 and 12 cycles respectively.

### Sequencing Data Pre-Processing and Analysis

The scRNA-seq sequencing libraries were sequenced on a NovaSeq S4 lane. The sequencing reads were mapped to the custom-made pre-mRNA reference transcriptome, and gene-cell matrices were generated via the 10× Genomics Cell Ranger pipeline as previously described. Subsequent gene expression analyses were conducted in R (4.1.2) using Seurat (4.0.3) as previously described. Cells were filtered if possessing fewer than 1,000 or more than 45,000 unique transcripts, or more than 15% of mitochondrial transcripts. Transcriptomic cell types were identified and cell clusters co-expressing two or more markers for canonical cell classes were excluded. Both sodium depletion and sated conditions included two trials collected from different time points for the consistency of the data. Four data sets were merged into a single-gene expression-matrix and canonical correlation analysis was applied. This yielded an SFO data set of 7819 neurons (four sets merged). Dimensionality reduction was performed with 25 principal components for major cell classes and 10 to 13 principal components for neural subtypes. The resolution of clustering was 0.8 for all cell classes and 0.6-2 for neural subtypes. Canonical correlation analysis was performed to adjust the misalignment of transcriptomic neuron types due to physiological-state-derived transcriptional changes. Gene expression was normalized with identified integration anchors based on the top 200 differentially expressed gene sets from the untreated neural data set. For FIG. 9B, neuron matrix from sated- and sodium-depleted states were analyzed together with the previous hypovolemia neuron matrix.

### Spatial Transcriptomics

SeqFISH gene panel design: A custom seqFISH panel was designed to incorporate canonical cell-type-marker genes based on our scRNA-seq dataset analysis. The panel contained 192 genes of which 174 were detected using barcoded seqFISH imaging and 18 were identified serially via single-molecule FISH. The panel was custom ordered by Spatial Genomics, Inc.

SeqFISH sample collection and preparation: Adult (8-12 weeks old) C57BL/6J male mice under sated or sodium depleted states were euthanized (n=5 mice per state). Each brain was dissected, freshly embedded in Tissue-Tek O.C.T. Compound (Sakura, 4583), and then flash-frozen in liquid nitrogen. Cryosections of 10-μm thickness containing the SFO were cut and mounted onto treated coverslips. Immediately post-collection, sections were fixed in fresh 4% paraformaldehyde (Thermo Scientific, 28908) for 15 min at room temperature. After rinsing three times in 1×PBS for 5 min each, sections were dehydrated using 70% ethanol for 30 sec at room temperature. The sections were air-dried at room temperature for approximately 15 min and stored at −80 C. SeqFISH experiments were performed using the seqFISH+ protocol with some modifications at Spatial Genomics, Inc. Briefly, the fixed tissue sections underwent permeabilization in 70% ethanol followed by clearing, rinsing, and air-drying steps. The coverslip containing each section was assembled into Spatial Genomics custom flow cells. The flow cells were then hybridized with the seqFISH primary probe panel and incubated in a humidified chamber at 37° C. for 24 hr. After hybridization, samples were washed with buffers for subsequent imaging.

seqFISH imaging via Gene Positioning System: Imaging was performed using the Gene Positioning System (GenePS, Spatial Genomics, Inc.), which enables automated image acquisition, reagent delivery, and data processing. The SFO was selected as a region of interest (ROI) for the experiment based on the brightfield and/or DAPI images. Automated experiment execution was initiated post-ROI selection. Each experiment proceeded through multiple rounds of decode probe hybridization, imaging, and signal removal until all the hybridization rounds were complete.

Image processing and analyses: Raw-image files were processed on-instrument to align images across multiple hybridization rounds and detect RNA fluorescent signals. The data were further analyzed using custom Spatial Genomics analysis software to decode the transcript identities and segment cells. Cell segmentation was performed using a machine learning algorithm based on the nuclear DAPI stain. The decoded RNA molecules were then assigned to individual cells, generating cell-by-gene count matrices and individual cell center coordinates for each ROI. For the downstream data analysis, cells that had unique gene counts of less than 10 were filtered. The images of the spatial distribution of the major cell types in the SFO with cell segmentation boundaries and color-coded cell type identities were directly generated by the Spatial Genomics analysis software. The spatial distribution of neurons was plotted according to cell center coordinates using Python (3.9.12). Individual neuron types were labeled with different colors based on raw transcripts of the neuronal subtype marker genes after manual curation. UMAP and violin plots were generated using Seurat (4.2.1) in R (4.1.1).

### RNAscope-Based Multicolour In Situ Hybridization

In situ hybridization was performed with the RNAscope Multiplex Fluorescent Assay versions 1 or 2 (Advanced Cell Diagnostics, 320850). Fixed-frozen brains from C57BL/6J, Ptger3^{Cre/wt} and Ptger3^{Cre/Cre} (equivalent to knockout) were prepared following the manufacturer's protocols. Following probes were applied to the samples: Cre (312281), Ptger3 (504481 and 501831), GFP (409011), and Pdyn (318771). The samples were imaged with confocal microscopy and visualized via z-stacks.

### Quantification and Statistical Analysis

No statistical methods were used to predetermine sample size. The experiments were not randomized, and investigators were not blinded to allocation during experiments and outcome assessment. Data are presented as means±SEMs in figures. Wilcoxon test, Mann-Whitney test, and one- or two-way ANOVA with post hoc tests were applied to decide the significance level. Significant threshold was maintained at α=0.05 (ns p>0.05, *p<0.05, **p<0.01, ***p<0.001).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/of” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges ther.eof Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of reducing aversive taste tolerance in a subject in need thereof, the method comprising: inhibiting a plurality of prostaglandin receptor type 3 (Ptger3)-positive neurons in the lamina terminalis (LT^{Ptger3}-neurons) of the subject, thereby reducing aversive taste tolerance in the subject.

2. The method of claim 1, wherein reducing aversive taste tolerance in the subject comprises reducing ingestion of sodium, one or more aversive substances, or a combination thereof by the subject.

3. (canceled)

4. The method of claim 2, wherein the sodium is at a concentration of greater than 300 mM; or
wherein reducing aversive taste tolerance in the subject comprises reducing ingestion of the sodium and the one or more aversive substances by the subject, and wherein the sodium is at a concentration of less than 120 mM.

5. (canceled)

6. The method of claim 2, wherein the one or more aversive substances comprises one or more aversive minerals, one or more bitter-tasting substances, one or more sour-tasting substances, or any combination thereof.

7.-12. (canceled)

13. The method of claim 1, wherein the plurality of LT^{Ptger3}-neurons are in the subfornical organ of the LT (SFO^{Ptger3}-neurons), the vascular organ of the LT (OVLT^{Ptger3}-neurons), or both.

14. (canceled)

15. (canceled)

16. The method of claim 1, wherein inhibiting the plurality of LT^{Ptger3}-neurons of the subject comprises administration of a Ptger3-inhibitor to the subject.

17. (canceled)

18. The method of claim 16, wherein the Ptger3-inhibitor inhibits activity of a prostaglandin.

19. (canceled)

20. The method of claim 16, wherein the Ptger3-inhibitor is a small molecule, an antibody, or a nucleic acid.

21. The method of claim 20, wherein the nucleic acid is an anti-sense RNA comprising a microRNA (miRNA), a precursor microRNA (pre-miRNA), a small interfering RNA (siRNA), a short-hairpin RNA (shRNA), precursors thereof, derivatives thereof, or a combination thereof.

22. (canceled)

23. The method of claim 20, wherein the nucleic acid is complexed or associated with one or more lipids or lipid-based carriers, thereby forming liposomes, lipid nanoparticles (LNPs), lipoplexes, and/or nanoliposomes.

24. The method of claim 20, wherein the nucleic acid comprises, or further comprises, one or more vectors, wherein at least one of the one or more vectors is a viral vector, a plasmid, a transposable element, a naked DNA vector, a lipid nanoparticle (LNP), or any combination thereof.

25. The method of claim 16, wherein the Ptger3-inhibitor is an anti-inflammatory agent.

26. (canceled)

27. (canceled)

28. The method of claim 1, wherein inhibiting the plurality of LT^{Ptger3}-neurons of the subject comprises optogenetic inhibition or chemogenetic inhibition.

29.-37. (canceled)

38. A method of increasing aversive taste tolerance in a subject in need thereof, the method comprising: stimulating a plurality of prostaglandin receptor type 3 (Ptger3)-positive neurons in the lamina terminalis (LT^{Ptger3}-neurons) of the subject, thereby increasing aversive taste tolerance in the subject.

39. The method of claim 38, wherein increasing aversive taste tolerance in the subject comprises increasing ingestion of sodium, one or more aversive substances, or a combination thereof by the subject.

40. (canceled)

41. The method of claim 39, wherein the sodium is at concentration of greater than 300 mM, or
wherein increasing aversive taste tolerance in the subject comprises increasing ingestion of the sodium and the one or more aversive substances by the subject, and wherein the sodium is at a concentration of less than 120 mM.

42. (canceled)

43. The method of claim 39, wherein the one or more aversive substances comprises one or more aversive minerals, one or more bitter-tasting substances, one or more sour-tasting substances, or any combination thereof.

44.-49. (canceled)

50. The method of claim 38, wherein the plurality of LT^{Ptger3}-neurons are in the subfornical organ of the LT (SFO^{Ptger3}-neurons), the vascular organ of lamina terminalis (OVLT^{Ptger3}-neurons), or both.

51. (canceled)

52. (canceled)

53. The method of claim 38, wherein stimulating the plurality of LT^{Ptger3}-neurons of the subject comprises administration of a Ptger3-activator to the subject, or
wherein stimulating the plurality of LT^{Ptger3}-neurons of the subject comprises optogenetic stimulation or chemogenetic stimulation.

54.-68. (canceled)

69. A method of identifying a modulator of aversive taste tolerance, the method comprising:
(a) contacting a candidate compound with a plurality of prostaglandin receptor type 3 (Ptger3)-positive neurons in the lamina terminalis (LT^{Ptger3}-neurons) to determine an electrophysiological response in the LT^{Ptger3}-neurons;
(b) identifying the candidate compound as a modulator of the LT^{Ptger3}-neurons if the electrophysiological response in the LT^{Ptger3}-neurons contacted with the candidate compound is altered as compared to the electrophysiological response in the LT^{Ptger3}-neurons prior to contacting with the candidate compound;
(c) administering the identified modulator of the LT^{Ptger3}-neurons to a subject;
(d) assessing the change in valence toward sodium and/or one or more aversive substances of the subject in response to the administration of the identified modulator of the LT^{Ptger3}-neurons; and
(e) identifying the identified modulator of the LT^{Ptger3}-neurons as a modulator for aversive taste tolerance if the identified modulator of the LT^{Ptger3}-neurons changes the valence toward sodium and/or one or more aversive substances of the subject compared to a control.

70.-76. (canceled)
